(19) United States
(12) Patent Application Publication
(10) Pub. No.: US 2004/0018508 A1
(43) Pub. Date: Jan. 29, 2004

(54) Surrogate antibodies and methods of preparation and use thereof

(75) Inventor: Stephen B. Friedman, Chapel Hill, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Syntherica Corporation, Durham, NC (US)

(21) Appl. No.: 10/370,052

(22) Filed: Feb. 19, 2003

Related U.S. Application Data
(60) Provisional application No. 60/358,459, filed on Feb. 19, 2002.

(51) Int. Cl. C12Q 1/68; C07K 9/00; C07H 21/02; C07H 21/04

(52) U.S. Cl. 435/6; 536/23.1; 530/322

(57) ABSTRACT

A process is described for producing surrogate antibody molecules that mimic the structure, stability, and binding characteristics of a natural antibody. Surrogate antibody structure, composition of surrogate antibody libraries, methods of surrogate antibody preparation, and surrogate antibody applications are disclosed. Also disclosed are methods of surrogate antibody structural stabilization and resistance to nucleases. The surrogate antibodies comprise a specificity strand and a stabilization strand. The specificity strand comprises a nucleic acid sequence having a specificity region flanked by a first constant region and a second constant region. The stabilization strand comprises a first stabilization region that interacts with the first constant region and a second stabilization region that interacts with the second constant region. In further embodiments, the stabilization strand and the specificity strand comprise distinct molecules. In other embodiments, the surrogate antibody molecules may comprise polyoligonucleotides that have at least one nucleotide sequence that forms a loop with specific ligand-binding properties. Surrogate antibody libraries containing a large population of random binding molecules are pre-assembled and used in a process that captures and amplifies those molecules having prerequisite binding characteristics. The amplified surrogate antibody molecule produced by the process has identical structure and binding characteristics to the parent molecule captured from the initially assembled library. Surrogate antibody molecules contain binding loop(s) that are formed and stabilized by the hybridization of at least two adjacent and juxtaposed strands, one strand having a greater number of nucleotides than the other. The preparation of a polyclonal surrogate antibody reagent proceeds through phases of capture/enrichment and amplification, specificity enhancement, and affinity enhancement. Depending upon the intended application, polyclonal surrogate antibody reagents can be processed to monoclonality. These molecules expand upon the binding characteristics of natural immunoglobulins, and do not require animals, animal facilities, cell culture or the stimulation of an immune response, in their development. They can be used as an effective replacement for natural antibody molecules, and therefore can be used in testing methods like immunoassay, as therapeutic agents, for specific labeling, and for research purposes. Targets ligands compatible with the development of surrogate antibodies include compounds, organisms, and cells that when complexed to a surrogate antibody in solution attain characteristics that can be physically or chemically differentiated from uncomplexed surrogate antibody.

### CROSS-REFERENCE TO RELATED APPLICATIONS

The application claims priority to U.S. Provisional Application No. 60/358,459 filed Feb. 19, 2002, which is herein incorporated by reference.

### FEDERALLY SPONSORED RESEARCH OR DEVELOPMENT

This application has received government assistance from National Institute of Health Grant No. 2-R44-ES010534-02.

### FIELD OF THE INVENTION

This invention relates to surrogate antibodies and methods of preparation and use thereof. The surrogate antibodies (SAbs) are useful for any purpose to which a binding reaction can be put, for example in assay methods, diagnostic procedures, cell sorting, as inhibitors of target molecule function, as probes, as sequestering agents and the like. The surrogate antibodies can be used in the treatment, diagnosis and prophylaxis of disease, to identify new cancer markers, as substitutes for antibodies in antibody-based immunoassays, and to identify environmental contaminants. In addition, the antibodies can have catalytic activity. Target molecules include natural and synthetic polymers, including proteins, polysaccharides, glycoproteins, hormones, receptors and cell surfaces, and small molecules such as drugs, metabolites, co-factors, transition state analogs, toxins, and environmental contaminants.

### BACKGROUND OF THE INVENTION

Antibodies are generated in the body as part of the immune system and are used to treat a variety of diseases. Antibodies are also used in antibody-based immunoassays to identify the presence of various compounds that are bound selectively by the antibodies. A limitation of antibody-based immunoassays is that a significant amount of time is required to produce, identify, and characterize appropriate antibodies. It is difficult to prepare high-throughput assays that require the development of a large number of antibodies to simultaneously screen for a plurality of targets.

Antibodies generated in animals using immunogens are used to treat a variety of human diseases. However, animal antibodies are foreign to the human immune system and stimulate an xenogenic anti-antibody response that facilitates their elimination and limits their effectiveness. This limitation can often be overcome by preparing humanized antibodies, but this is a laborious and time-consuming process. In general, monoclonal antibodies offer selectivity, and polyclonal antibodies offer greater sensitivity. However, it is typically difficult to produce a single antibody composition that has both of these properties. A further limitation of antibodies is their maximum binding cavity size, and a repertoire of binding specificities that is limited by evolution and the host genome. That, coupled with the fact that antibody molecules are immunogenic proteins that require extensive development time to produce limits their use in a variety of applications.

Nucleic acids are known to form secondary and tertiary structures in solution. The double-stranded forms of DNA include the so-called B double-helical form, Z-DNA and superhelical twists (Rich et al. (1984) Ann. Rev. Biochem. 53: 791-846). Single-stranded RNA forms localized regions of secondary structure such as hairpin loops and pseudoknot structures (Schimmel (1989) Cell 58:9-12). However, little is known concerning the effects of unpaired loop nucleotides on stability of loop structure, kinetics of formation and denaturation, thermodynamics, and almost nothing is known of tertiary structures and three dimensional shape, nor of the kinetics and thermodynamics of tertiary folding in nucleic acids (Tuerk et al. (1988) Proc. Natl. Acad. Sci. USA 85:1364-1368). Poly-oligonucleotide structures that function as surrogate antibodies have not been previously described in the literature.

It would be advantageous to have specific, non-immunogenic, high affinity surrogate antibodies that could be produced rapidly. The present invention provides such surrogate antibodies.

### SUMMARY OF THE INVENTION

Surrogate antibodies, libraries of surrogate antibodies, methods for making the surrogate antibodies, and assay methods using the antibodies and libraries thereof are disclosed. Also disclosed are methods for stabilizing the antibodies with respect to nucleases. Further, therapeutic methods using the antibodies, alone, in combination with other therapeutics, or conjugated to therapeutics, are also disclosed. Surrogate antibody molecules having single or multiple labels per binding molecule are also disclosed.

The surrogate antibodies comprise one or more specificity strand(s) and a stabilization strand. The specificity strand comprises a nucleic acid sequence having a specificity region flanked by a first constant region and a second constant region. The stabilization strand comprises a first stabilization region that interacts with the first constant region and a second stabilization region that interacts with the second constant region. In further embodiments, the stabilization strand and the specificity strand comprise distinct molecules.

In one embodiment, the surrogate antibodies are molecules that possess a random loop structure (specificity region) within a hybridized structure comprising at least two strands that hybridize to each other and stabilize the loop structure. When the strands are hybridized together, under ligand-binding conditions (length and extent of hybridization can be tailored to the binding conditions necessary for ligand-surrogate antibody interaction), they form an annealed hybridized strand with a loop structure.

Each surrogate antibody within an assembled surrogate antibody library has a unique specificity region sequence and can potentially bind to a target molecule. Libraries of the pre-formed antibodies can be screened to find the antibodies that bind specifically to a desired target compound or molecule. The invention is based on the observation that nucleic acids can be formed that interact in such a manner as to form stabilized loop structures. Loop structures can have the diversity associated with conventional antibodies or even greater diversity. The surrogate antibodies have sufficient chemical versatility to form specific binding pairs with virtually any chemical compound, whether monomeric or polymeric. Molecules of any size can serve as targets. In specific embodiments, e.g., for therapeutic applications, binding takes place in aqueous solution at conditions of salt, temperature, and pH at or near acceptable physiological limits.

The targets (ligands) can be screened to identify surrogate antibodies that bind to the targets. Assays, e.g., high throughput assays, can be used to determine the effect of binding of a surrogate antibody on the function of the target molecule or target cell. The method can be used to isolate and identify surrogate antibodies that bind to proteins, including both nucleic acid-binding proteins and proteins not known to bind nucleic acids as part of their biological function. The method can be used to detect the presence or absence of, and/or measuring the amount of a target molecule in a sample. Alternatively, the method can be used to identify target molecules that are present in one type of cell/tissue/organ versus another type of cell/tissue/organ. The presence of the target molecule is determined by its binding to a surrogate antibody specific for that target molecule.

Ligand-binding surrogate antibodies can be isolated in the starting library by incubating the library with a target ligand and filtering through a membrane having a porosity that excludes the target ligand and target ligand-surrogate antibody complex while allowing unbound surrogate antibodies to pass into the filtrate.

The surrogate antibodies described herein can be used in diagnostic methods in a manner similar to conventional antibody-based diagnostics. The surrogate antibodies can be used to specifically deliver a pharmaceutical agent to a specific site on or within a cell, tissue, organ, or organ system, to specifically detect, a target ligand on or within a cell, tissue, organ, or organ system, to deliver multiple therapeutic agents specifically to a target site, and/or amplify the sensitivity of a detection method by incorporating multiple reporter molecules. The surrogate antibodies that bind to small molecule targets can be used as diagnostic assay reagents and therapeutically as sequestering agents, drug delivery vehicles, and modifiers of hormone action. Synthetic catalytic antibodies can be selected, based on binding affinity and the catalytic activity of the antibodies once bound. One way to select for catalytic antibodies is to search for surrogate antibodies that bind to transition state analogs of an enzyme catalyzed reaction.

Surrogate antibodies can also be prepared to specifically bind toxic organic compounds, such as PCBs (polychlorinated biphenyls). They can be used to develop rapid, cost-effective, testing arrays that can provide a profile of contamination in a soil, water, or air sample, or be used to remove contamination in environmental remediation.

Surrogate antibodies with differing specificity regions and/or cavity sizes and/or conformations can be used in sensitivity, specificity and affinity maturation rounds. In one embodiment, each of the separate populations of molecules is labeled with unique 5′ and/or 3′ end label(s) for easy detection. The process allows for the identification of optimal binding cavity size and conformation as provided by nucleotide sequence.

The function of target molecules can be modified or modulated by the binding of surrogate antibodies. For example, surrogate antibodies when bound can inhibit or activate the function of molecules such as receptors, effectors, enzymes, hormones, and transport proteins.

Accordingly, in one aspect, the invention relates to a surrogate antibody molecule comprising a first oligonucleotide strand and a second oligonucleotide strand. The first strand comprises two adjacent stabilization regions that hybridize to the second strand under predetermined conditions. The second strand comprises a specificity region that does not hybridize to the first strand. The specificity region is flanked by stabilization regions that hybridize to the stabilization regions of the first stand under the predetermined conditions.

The invention also includes aspects that involve more complex surrogate antibody structure involving more than one first strand or second strand, or more than one of each.

Accordingly, in another aspect, the invention relates to a surrogate antibody molecule comprising at least one first oligonucleotide strand and at least one second oligonucleotide strand. The first strand comprises stabilization regions that hybridize to the second strand under predetermined conditions. The second strand comprises at least one specificity region that does not hybridize to the first strand. At least one specificity region is flanked by stabilization regions that hybridize to the stabilization regions of the first strand under the predetermined conditions. According to the compositions and methods of the invention, the stabilization region nucleotide sequences may be varied to allow for directed hybridization or interaction and structure customization of the assembled molecule.

In yet another aspect, the invention relates to a surrogate antibody molecule comprising a first oligonucleotide strand and at least one-second oligonucleotide strand. The first strand comprises stabilization regions that hybridize to the second strands under predetermined conditions. At least one second strand comprises at least two specificity regions that do not hybridize to the first strand. The specificity regions are flanked by stabilization regions that hybridize to the stabilization regions of the first strand under the predetermined conditions.

In still another aspect, the invention relates to a surrogate antibody molecule comprising a first oligonucleotide strand and at least two second oligonucleotide strands. The first strand comprises stabilization regions that hybridize to the second strands under predetermined conditions. The second strands each comprise at least one specificity region that does not hybridize to the first strand. The specificity regions are flanked by stabilization regions that hybridize to the stabilization regions of the first stand under the predetermined conditions. The first strand can also comprise at least one specificity region.

The surrogate antibody molecules of the invention can comprise spacer regions that reduce bond stress. The spacer regions can be on the first strand adjacent to a specificity region of the second strand. The spacer regions can also be on the first strand between adjacent stabilizations regions that hybridize to two adjacent second strands.

Each stabilization region can comprise from about 2 to about 100 nucleotides, from about 5 to about 90 nucleotides, or from about 10 to about 30 nucleotides. The stabilization regions of the molecule allow stable hybridization between strands under predetermined conditions, the predetermined conditions being those conditions necessary for binding of a target ligand to the specificity regions. The hybridization of the stabilization regions allows a binding loop(s) to be formed by the stress created by the hybridization of strands of dissimilar size. The specificity region(s) can comprise from about 2 to about 100 nucleotides. The specificity region(s) can also comprise from about 10 to about 60 nucleotides, from about 10 to about 80 nucleotides, or from about 10 to about 40 nucleotides.

The first strand can be a naturally occurring oligonucleotide strand comprising naturally occurring base modifications that provide nuclease protection and/or immune tolerance. Further, the stabilization regions of the second strand can be naturally occurring oligonucleotide sequences comprising naturally occurring modifications that provide nuclease protection and/or immune tolerance.

The two strands of the surrogate antibodies of the invention can be RNA, DNA, TNA, amino acids, or any combination thereof (i.e., RNA-RNA, DNA-DNA, TNA-RNA, TNA-DNA, RNA-DNA, DNA-amino acid, TNA-amino acid, or RNA-amino acid ect).

The surrogate antibody can comprise at least one moiety selected from the group consisting of a reporter molecule, a linking molecule, an enzyme, and a therapeutic agent. At least one moiety can be affixed to a stabilization region.

The invention also provides a process for producing surrogate antibodies, including processes for generating increased affinity/sensitivity and specificity.

Accordingly, in one aspect, the invention relates to a process for producing a surrogate antibody by preparing a first oligonucleotide strand comprising stabilization regions that hybridize to a second oligonucleotide strand, wherein the two strands are of unequal length with the first strand having fewer nucleotides in sequence than the second strand. A library is prepared of second oligonucleotide strands comprising at least one specificity region comprising a variable sequence of nucleotides and comprising stabilization regions flanking the specificity region that hybridize to the stabilization regions of the first oligonucleotide strand. The first and second oligonucleotide strands are combined such that the stabilization regions of the second oligonucleotide strands are hybridized (in a predetermined way based upon sequence alignment) to the stabilization regions of the first oligonucleotide strands to form a surrogate antibody. The hybridized strands are contacted with a target ligand and the target ligand and any bound surrogate antibodies are separated from unbound surrogate antibodies. The second oligonucleotide strands bound to the target ligand are amplified. The amplified second oligonucleotide strands are purified and hybridized to the first oligonucleotide strand to form the surrogate antibody.

The invention also provides methods of increasing specificity of the surrogate antibodies after the initial process steps leading to the amplification and formation of the surrogate antibody preparation.

Accordingly, in another aspect, the process of the invention further comprises contacting the surrogate antibody with a target hapten; incubating the surrogate antibody and hapten with a hapten-protein conjugate; separating surrogate antibody bound to the hapten from the hapten-protein conjugate and any surrogate antibody bound thereto; amplifying the second oligonucleotide strand of any surrogate antibodies bound to the hapten; purifying the amplified second oligonucleotide strands; and hybridizing the amplified second oligonucleotide strand with the first oligonucleotide strand to form the surrogate antibody. The separation step can comprise using a filter that retains the protein-hapten conjugate, while allowing the surrogate antibody, the hapten, and any bound complexes of the surrogate antibody and unconjugated hapten to pass into the filtrate. The specificity of preparation can also be increased by including steps involving the incubation of surrogate antibody preparations with potentially cross-reactive ligands (a non-specific moiety) that may be present along with a target ligand. In each variation of these methods, specificity is increased using separation and amplification methods are described herein. The order of steps involved in preparing a surrogate antibody of increased specificity can be varied, and may be carried out in accordance with a particular need associated with the intended use of the surrogate antibody.

The invention also provides for increasing the binding affinity/sensitivity of the surrogate antibody preparations after the initial process steps leading to the amplification and formation of the surrogate antibody preparation.

Accordingly, in another aspect, the process of the invention further comprises the steps of contacting the surrogate antibody with the target ligand under conditions that reduce binding affinity (e.g., agents that deteriorate hydrophobic, hydrogen, electrostatic, Van der Waals interactions); separating the target ligand and any bound surrogate antibodies from unbound surrogate antibodies; amplifying the second oligonucleotide strands bound to the target ligand; purifying the amplified second oligonucleotide strands; and hybridizing the amplified second oligonucleotide strand with the first oligonucleotide strand to form the surrogate antibody.

In order to increase sensitivity, the process of the invention can also further comprise the steps of contacting the surrogate antibody with the target ligand at lower concentrations than a concentration used to contact the surrogate antibody prior to an initial amplification step; separating the target ligand and any bound surrogate antibodies from unbound surrogate antibodies; amplifying the second oligonucleotide strands bound to the target ligand; purifying the amplified second oligonucleotide strands; and hybridizing the amplified second oligonucleotide strand with the first oligonucleotide strand to form the surrogate antibody.

The invention provides for the production of a polyclonal or a monoclonal surrogate antibody preparation. The process as described above generally results in a polyclonal preparation wherein multiple surrogate antibodies having individual specificity regions are selected and amplified. The invention further provides for the production of a monoclonal surrogate antibodies. These steps involve the amplification and cloning of second oligonucleotide strand sequences produced according the foregoing processes, followed by clonal selection and evaluation as described herein.

### BRIEF DESCRIPTION OF THE FIGURES

FIG. 1 is a diagram representing a surrogate antibody (SAb) molecule that contains one or more stabilization regions (ST) composed of juxtaposed oligonucleotide strands (A, A′, D, and D′) that border one or more specificity regions (SP) composed of a sequence of nucleotides that form a ligand-binding cavity. In this embodiment, the upper stand (specificity strand) comprises a specificity region (SP) flanked by two constant regions (A and D). The lower strand (stabilization strand) comprises a spacer region flanked by two stabilization regions (A′ and D′) that interact with the respective constant region (A and D).

FIGS. 2A and 2B are diagrams representing two embodiments of surrogate antibody molecules that include multiple specificity regions (SP region loops), stabilization regions (ST), and spacer regions (S).

FIGS. 3A-3D are diagrams representing four embodiments of surrogate antibody molecules that contain multiple specificity regions (SP region loops), stabilization regions (ST), and spacer regions (S) and that collectively provide multi-dimensional ligand binding.

FIG. 4 is a schematic illustration showing the binding of target ligands to surrogate antibody molecules containing SP region loops of varying sizes.

FIG. 5 is a schematic illustration showing surrogate antibody capacity to enhance binding affinity and specificity relative to natural antibodies.

FIG. 6 is a schematic illustration of one method of preparing surrogate antibodies.

FIG. 7 provides a non-limiting method for amplifying a surrogate antibody. In this embodiment, “F48” comprises the stabilization strand (SEQ ID NO: 1) and “F22-40-25 (87)” comprises the specificity strand (SEQ ID NO: 2). The stabilization strand comprises a 5 nucleotide mis-match (shaded box) to the specificity strand. This mis-match in combination with the appropriate primers (B21-40, SEQ ID NO:3; and F17-50, SEQ ID NO:4) will prevent amplification of the stabilization strand during PCR amplification. More details regarding this method are found in Example 4.

FIG. 8 illustrates the electrophoretic mobility of the surrogate antibody that were assembled using different combinations of specificity and stability primers.

FIG. 9 characterizes the surrogate antibodies using a denaturing gel to verify the duplex nature of the molecule.

FIG. 10 illustrates the selection and enrichment of the surrogate antibodies to the BSA-PCT (BZ101 congener) conjugate through 8, 9 and 10 cycles. Signal/Negative control represents as a percent, the amount of surrogate antibody bound to the target verses the amount of surrogate antibody recovered when the target is absent (negative control).

FIG. 11 illustrates the unique congener response profiles the array would produce for selected Aroclors®.

FIG. 12 illustrates the selection and enrichment of the surrogate antibodies to IgG. Signal/Negative control represents as a percent, the amount of surrogate antibody bound to the target verses the amount of surrogate antibody recovered when the target is absent (negative control).

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings.

Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

Surrogate antibodies, libraries of surrogate antibodies, methods for making the surrogate antibodies, and assay methods using the antibodies and libraries thereof are disclosed. Also disclosed are methods for stabilizing the antibodies with respect to nucleases. Further, therapeutic methods using the antibodies, alone, or conjugated to therapeutic agents, are also disclosed.

Compositions

I. Surrogate Antibodies

Compositions comprising surrogate antibody molecules and libraries containing the surrogate antibody molecules are provided. Further provided are surrogate antibodies bound to their ligands. As used herein, a surrogate antibody refers to a class of molecules that contain discrete nucleic acid structures or motifs that enable selective binding to target molecules. More specifically, a surrogate antibody possesses a random loop structure (i.e., a specificity region) and the appropriate structural elements that allow for the stabilization of the loop structure.

The vast number of sequences and shapes possible for the binding loop(s) (i.e., specificity regions) of the surrogate antibodies will conceivably allow, especially with sequences and modified nucleotides never tested during evolutionary history, every desired function and binding affinity even though conventional oligonucleotides are comprised of only four nucleotides and have a backbone that is highly charged. That is, the surrogate antibodies are capable of having appropriate diversity in the loop-forming specificity region(s) to provide sufficient physical and chemical diversity for the tight and specific binding to most targets. Appropriately formed libraries of surrogate antibodies are believed to consist of molecules that collectively equal or exceed the binding diversity observed in the binding molecules of the vertebrate immune system. While antibody molecules produced by the humoral immune response can bind many ligands, the surrogate antibody libraries of the present invention can provide equal or superior opportunities because the binding site of a surrogate antibody is not restricted in size and production is not limited by genome composition and expression in an organism. The libraries can include such vast numbers of different structures that whatever intrinsic advantages naturally occurring antibodies can have is offset by the vastness of the possible “pool” from which the surrogate antibodies can be selected and the versatility of the binding sites that can be produced.

The diverse structures of the surrogate antibodies of the present invention, along with the diverse range of binding specificities, binding affinities, and methods of producing such compositions are described in further detail below.

In one embodiment, the surrogate antibody comprises a first strand, referred to herein as the “specificity strand”, and a second strand referred to herein as the “stabilization strand”. In this embodiment, the specificity strand comprises a nucleic acid sequence having a specificity region flanked by a first constant region and a second constant region. The stabilization strand comprises a first stabilization region that interacts with the first constant region and a second stabilization region that interacts with the second constant region.

The invention encompasses isolated or substantially isolated surrogate antibody compositions. An “isolated” surrogate antibody molecule is substantially free of other cellular material, or culture medium, chemical precursors, or other chemicals when chemically synthesized. A surrogate antibody that is substantially free of cellular material includes preparations of surrogate antibody having less than about 30%, 20%, 10%, 5%, (by dry weight) of contaminating protein or nucleic acid. In addition, if the surrogate antibody molecule comprises nucleic acid sequences homologous to sequences in nature, the “isolated” surrogate antibody molecule is free of sequences that may naturally flank the nucleic acid (i.e., sequences located at the 5′ and 3′ ends of the nucleic acid) in the genomic DNA of the organism from which the surrogate antibody has homology.

As used herein, nucleic acid means TNA, DNA, RNA, single-stranded or double-stranded, and any chemical modifications thereof. A surrogate antibody can be composed of double-stranded RNA, single-stranded RNA, single stranded DNA, double stranded DNA, a hybrid RNA-DNA double strand combination, a hybrid TNA-DNA, a hybrid TNA-RNA, a hybrid amino acid/RNA, amino acid/DNA, amino acid/TNA, or any combination thereof provided there exists interacting regions that allow for the stabilization of one or more loop structures (i.e., specificity domains). A more detailed description of these diverse antibody structures is provided below. Specific antibodies can be captured and identified using the methods described herein and amplified in large amounts once identified. It is further recognized that the nucleic acid sequences include naturally occurring nucleotides and synthetically modified nucleotides.

A. The Specificity Strand

As used herein, the specificity strand of the surrogate antibody comprises a nucleic acid molecule having a specificity region flanked by two constant regions. As used herein “flanked by” is intended that the constant regions are immediately adjacent to the specificity region or, alternatively, the constant regions are found 5′ and 3′ to the specificity region but separated by a spacer sequence. The specificity region functions as a ligand binding cavity, while the constant domains interact with the stabilization domains found on the stabilization strand to thereby allow the specificity domain to form a ligand binding cavity.

The specificity strand comprises a nucleic acid sequence composed of ribonucleotides, modified ribonucleotides, deoxyribonucleotides, modified deoxyribonucleotides, (3′,2′)-α-L-threose nucleic acid (TNA), modified TNA, or any combination thereof. A modification includes the attachment (any means of interaction, i.e., covalent, ionic, ect, that is stable under the desired conditions) of any functional moiety or molecule to the nucleotide sequence. See, for example, Chaput et al. (2003) J. Am. Chem. Soc. 125:856-857, herein incorporated by reference. The modification can be at the 5′ end and/or the 3′ end of the sequence, added to individual nucleotide residues anywhere in the strand, attached to all or a portion of the pyrimidines or purine residues, or attached to all or a portions of a given type of nucleotide residue. While various modifications to DNA and RNA residues are known in the art, examples of some modifications of interest to the surrogate antibodies of the present invention are discussed in further detail below.

The specificity strand and its respective domains (i.e., the constant domains and the specificity domains and, in some embodiments, a spacer regions) can be of any length, so long as the strand can form a surrogate antibody as described elsewhere herein. For example, the specificity strand can be between about 10, 50, 100, 200, 400, 500, 800, 1000, 2000, 4000, 8000 nucleotides or greater in length. Alternatively, the specificity strand can be from about 15-80, 80-150, 150-600, 600-1200, 1200-1800, 1800-3000, 3000-5000 or greater. The constant domains and the specificity domains can be between about 2 nucleotides to about 100 nucleotides in length, between about 20 to about 50 nucleotides in length, about 10 to about 90 nucleotides in length, about 10 to about 80 nucleotides in length, about 10 to about 60 nucleotides in length, or about 10 to about 40 nucleotides in length.

While a surrogate antibody molecule does not require a spacer region in the specificity region, if the region is present it can be of any length. For example, if a spacer region is present in the specificity strand, this region can be about 2 nucleotides to about 100 nucleotides in length, between about 20 to about 50 nucleotides in length, about 10 to about 90 nucleotides in length, about 10 to about 60 nucleotides in length, or about 10 to about 40 nucleotides in length. In yet other embodiments, the spacer region need not comprise a nucleic acid residue but could be any molecule, such as a phosphate moiety, incorporated into the strand that provides the desired spacing to form the surrogate antibody molecule.

In some embodiments, the specificity strand or its components (the constant regions or the specificity region) have significant similarity to naturally occurring nucleic acid sequences. In other embodiments, the nucleic acid sequence can share little or no sequence identity to sequences in nature. In still other embodiments, the nucleic acid residues may be modified as described elsewhere herein.

B. The Stabilization Strand

The surrogate antibody further comprises a stabilization strand. The stabilization strand comprises any molecule that is capable of interacting with the constant domains of the specificity strand and thereby stabilize the ligand-binding cavity of the specificity domain. Accordingly, the stabilization strand can comprise, for example, an amino acid sequence, a nucleic acid sequence, or various polymers including any cationic polymer, a cyclodextrin polymer, or a polymer having an appropriately charged intercalating agent, such as lithium bromide or ethidium bromide.

It is recognized that the stabilization regions in a surrogate antibody can be identical (i.e., the same nucleotide sequence or peptide sequence) or the regions can be non-identical, so long as each stabilization region interacts with their corresponding constant region in the specificity strand. In addition, the interaction between the constant regions and the stabilization regions may be direct or indirect. The interaction will further be such as to allow the interaction to occur under a variety of conditions including under the desired ligand-binding conditions.

In some embodiments, components of the surrogate antibodies (i.e., the stabilization strand and its respective domains) are not naturally occurring in nature. In others embodiments, they can have significant similarity to a naturally occurring nucleic acid sequences or amino acid sequences or may actually be naturally occurring sequences. One of skill in the art will recognize that the length of the stabilization domain will vary depending on the type of interaction required with the constant domains of the specificity strand. Such interactions are discussed in further detail elsewhere herein.

A stabilization strand comprising an amino acid sequence may comprise any polypeptide that is capable of interacting with the nucleic acid sequence of the constant domains of the specificity strand. For example, amino acid sequences having DNA binding activity (i.e., zinc finger binding domains (Balgth et al. (2001) Proc. Natl. Acad. Sci. 98:7158-7163; Friesen et al. (1998) Nature Structural Biology, Tang et al. (2001) J. Biol. Chem. 276:19631-9; Dreier et al. (2001) J. Biol. Chem. 29466-79; Sera et al. (2002) Biochemistry 41:7074-81, all of which are herein incorporated by reference), helix-turn domains, leucine zipper motifs (Mitra et al. (2001) Biochemistry 40:1693-9) or polypeptides having lectin-activity may be used for one or more of the stabilization domains. Accordingly, various polypeptides could be used, including transcription factors, restriction enzymes, telomerases, RNA or DNA polymerases, inducers/repressors or fragments and variants thereof that retain nucleic acid binding activity. See for example, Gadgil et al.(2001) J. Biochem. Biophys. Methods 49: 607-24. In other embodiments, the stabilization strand could include sequence-specific DNA binding small molecules such as polyamides (Dervan et al. (1999) Current Opinion Chem. Biol. 6:688-93 and Winters et al. (2000) Curr Opin Mol Ther 6:670-81); antibiotics such as aminoglycosides (Yoshhizawa et al. (2002) Biochemistry 41:6263-70) quinoxaline antibiotics (Bailly et al.(1998) Biochem Inorg Chem 37:6874-6883; AT-specific binding molecules (Wagnarocoski et al (2002) Biochem Biophys Acta 1587:300-8); rhodium complexes (Terbrueggen et al. (1998) Inorg. Chem. 330:81-7). One of skill in the art will recognize that if, for example, a zinc finger binding domain is used in the stabilization strand, the corresponding nucleic acid binding site will be present in the desired constant region of the specificity strand. Likewise, if a polypeptide having lectin-activity is used in the stabilization strand, the corresponding constant domain of the specificity strand will have the necessary modifications to allow for the desired interaction. When the stabilization domain comprises an amino acid sequence, any of the amino acid residues can be modified to contain functional moieties. Such modifications are discussed in further detail elsewhere herein.

When the stabilization strand comprises a nucleic acid molecule, the surrogate antibodies are formed from a first strand and a second strand. The first strand (the specificity strand), which as describe above, comprises a) two stabilization regions (referred to herein as constant regions) that are complementary to two stabilization regions on a second strand (the stabilization strand), and b) a specificity region that functions as a ligand-binding cavity located between the constant regions. The second strand (the stabilization strand) includes two stabilization regions complementary to the two stabilization regions (or constant regions) on the first strand (specificity strand). In one embodiment, the surrogate antibodies are formed when the first and second strands are hybridized together, where the specificity region forms a ligand-binding cavity that is not hybridized to any portion of the specificity strand. In this embodiment, the specificity strand is longer than the stabilization strand. In other embodiments, the ligand-binding cavity of the surrogate antibody can include one or more hairpin loops, asymmetric bulged hairpin loops, symmetric hairpin loops and pseudoknots.

The stabilization strand can comprise any nucleotide base, including for example, ribonucleotides, modified ribonucleotides, deoxyribonucleotides, modified deoxyribonucleotides or any combination thereof.

C. Forming a Surrogate Antibody

Methods of forming a surrogate antibody with the stabilization strand and the specificity strand are further provided. Methods of forming a surrogate antibody molecule comprise providing a specificity strand and a stabilization strand and contacting the specificity strand and the stabilization strand under conditions that allow for the first stabilization domain to interact with the first constant region and the second stabilization domain to interact with the second constant region. The specificity strand and stabilization strand can be contacting under any condition that allows for the stable interaction of the stabilization domains and the constant domains. This method of forming a surrogate antibody can be used to generate a population of surrogate antibodies.

As discussed below, conditions for forming the surrogate antibody molecule will vary depending on the ligand of interest and the intended applications. One of skill will be able to empirically determine the appropriate conditions for the desired application. For example, if the intended application is to occur under physiological conditions the formation of the antibody may be performed at pH 7.4 at a physiological salt concentration (i.e., 280-300 milliosmols).

When the stabilization strand comprises a nucleic acid sequence, the nucleotide sequences of the constant regions and the stabilization regions will be such as to allow for an interaction (i.e., hybridization) under the desired conditions (i.e., under ligand-binding conditions). Furthermore, the design of each stabilization domain and each constant domain will be such as to allow for assembly such that the first constant domain preferably interacts with the first stabilization domain and the second stabilization domain preferably interacts with the second constant domain. In this way, upon the interaction of the specificity strand and stabilization strand, sequence directed self-assembly of the surrogate antibody can occur.

In one embodiment, the surrogate antibody molecule is designed to result in a Tm for of each stabilization/constant domain interaction to be approximately about 15 to about 25° C. above the temperatures of the intended application (i.e., the desired ligand binding conditions). Accordingly, if the intended application is a therapeutic application or any application performed under physiological conditions, the Tm can be about 37° C.+about 15° C. to about 37° C.+25° C. (i.e., 49° C., 50° C., 52° C., 54° C., 55° C., 56° C., 58° C., 60° C., 62° C., 64° C., or greater). If the intended application is a diagnostic assay conducted at room temperature, the Tm can be 25° C.+about 15° C. to about 25° C.+about 25° C. (i.e., 38° C., 40° C., 41° C., 42° C., 43° C., 44° C., 46° C., 48° C., 50° C., 52° C., 53° C. or greater). Equations to measure Tm are known in the art. A preferred program for calculating Tm comprises the OligoAnalyzer 3.0 from IDT BioTools© 2000. It is recognized that any temperature can be used the methods of the invention. Thus, the temperature of the ligand binding conditions can be about 5° C., 1° C., 15° C., 16° C., 18° C., 20° C., 22° C., 24° C., 26° C., 28° C., 30° C., 32° C., 34° C., 38° C., 40° C., 42° C., 44° C., 46° C., 48° C., 50° C., 52° C., 54° C., 56° C., 58° C., 60° C. or greater.

Alternatively, the stabilization domains and the respective constant domains are designed to allow about 40% to about 99%, about 40% to about 50%, or about 50% to about 60%, about 60% to about 70%, about 70% to about 80%, about 85%, about 90%, about 95%, about 98% or more of the surrogate antibody population to remain annealed under the intended ligand binding conditions. Various methods, including gel electrophoresis, can be used to determine the % formation of the surrogate antibody. See Experimental section. In addition, calculation for this type of determination can be found, for example, in Markey et al. (1987) Biopolymers 26:1601-1620 and Petersteim et al. (1983) Biochemistry 22:256-263, both of which are herein incorporated by reference.

The relative concentration of the specificity strand and the stabilization strand can vary so long as the ratio will favor the formation of the surrogate antibody. Such conditions include providing an excess of the stabilization strand.

The constant regions and stabilization regions can have any desired G/C content, including for example about 0%, 5%, 10%, 15%, 20%, 25%, 30%, 35%, 40%, 45%, 50%, 55%, 60%, 65%, 70%, 75%, 80%, 85%, 90%, 95%, or 100% G/C.

The stabilization strand and the domains contained therein (stabilization domains and, in some embodiment, spacer domains) can be of any length, so long as the strand can form a surrogate antibody as described herein. For example, the stabilization strand can be between about 8, 10, 50, 100, 200, 400, 500, 800, 1000, 2000, 4000, 8000 nucleotides or greater in length. Alternatively, the stabilization strand can be from about 15-80, 80-150, 150-600, 600-1200, 1200-1800, 1800-3000, 3000-5000 or greater.

The stabilization domains can be between about 2 nucleotides to about 100 nucleotides in length, between about 20 to about 50 nucleotides in length, about 10 to about 90 nucleotides in length, about 10 to about 60 nucleotides in length, or about 10 to about 40 nucleotides in length. If a spacer region is present in the stabilization strand, this region can be about 1 nucleotide to about 100 nucleotides in length, between about 5 to about 50 nucleotides in length, about 10 to about 90 nucleotides in length, about 10 to about 60 nucleotides in length, or about 10 to about 40 nucleotides in length. Alternatively, as discussed elsewhere herein, the spacer can comprise one or more molecule including, for example, a phosphate moiety. The length and G/C content of each domain can vary so long as the interaction between the constant domains and the stabilization domain is sufficient to stabilize the antibody structure and produce a stable binding loop (specificity region). In addition, the stabilization strand can be linear, circular or globular and can further contain stabilization domains that allow for multiple (2, 3, 4, 5, 6, or more) specificity strands to interact.

The known oligonucleotide structures or motifs that are involved in non-Watson-Crick type interactions, such as hairpin loops, symmetric and asymmetric bulges, pseudo-knots and combinations thereof, have been suggested in the art to form from nucleic acid sequences of no more than 30 nucleotides. However, it has now been found that larger loop structures can be stabilized in the surrogate antibodies described herein. The specificity region can include between about 10 and 90 nucleotides, between about 10 and 80, between 10 and 60, or between 10 and 40 nucleotides. These stabilized binding cavities provide sites for hydrophobic binding and contribute to increased binding affinity in a manner that mimics the major force implicated in natural antibody binding. As such the ligand-binding cavity of the surrogate antibody can include one or more hairpin loops, asymmetric bulged hairpin loops, symmetric hairpin loops, and pseudoknots.

One of skill in the art will recognize that the stabilization domains and constant domains can be designed to maximize stability of the interactions under the desired conditions and thereby maintain the structure of the surrogate antibody. See, for example, Guo et al. (2002) Nature Structural Biology 9:855-861 and Nair et al. (2000) Nucleic Acid Research 28:1935-1940. Methods to measure the stability or structure of the surrogate antibody molecules are known. For example, surface plasmon resonance (BIACORE) can be used to determine kinetic values for the formation of surrogate antibody molecules (BIACORE AB). Other techniques of use include NMR spectroscopy and electrQphoretic mobility shift assays. See, Nair et al. (2000) Nucleic Acid Research 9:1935-1940. It is recognized that the complementary hybridizing stabilization regions and constant regions need not have 100% homology with one another. All that is required is that they bind together in a directed fashion and form a stable structure when exposed to ligand-binding conditions. Generally, this requires a stabilization domain and a constant domain having at least 80% sequence homology at least 90%, at least 95%, 96%, 97%, or 98% and higher sequence homology. In addition, the interaction may further require at least 5 consecutive complementary nucleotide residues in the stabilization domain and the corresponding constant domain.

By “sequence identity or homology” is intended the same nucleotides (or nucleotides with complementary bases) are found within the constant regions and the stabilization domain when a specified, contiguous segment of the nucleotide sequence of the constant domain is aligned and compared to the nucleotide sequence of the stabilization domain. Methods for sequence alignment and for determining identity between sequences are well known in the art. See, for example, Ausubel et al., eds. (1995) Current Protocols in Molecular Biology, Chapter 19 (Greene Publishing and Wiley-Interscience, New York); and the ALIGN program (Dayhoff (1978) in Atlas of Polypeptide Sequence and Structure 5:Suppl. 3 (National Biomedical Research Foundation, Washington, D.C.). With respect to optimal alignment of two nucleotide sequences, the contiguous segment of the constant/stabilization domain may have additional nucleotides or deleted nucleotides with respect to the corresponding constant/stabilization nucleotide sequence. The contiguous segment used for comparison to the reference nucleotide sequence will comprise at least 5, 10, 15, 20, 25 contiguous nucleotides and may be 30, 40, 50, 100, or more nucleotides. Corrections for increased sequence identity associated with inclusion of gaps in the nucleotide sequence can be made by assigning gap penalties.

The determination of percent identity between two sequences can be accomplished using a mathematical algorithm. Percent identity of a nucleotide sequence is determined using the Smith-Waterman homology search algorithm using a gap open penalty of 25 and a gap extension penalty of 5. Such a determination of sequence identity can be performed using, for example, the DeCypher Hardware Accelerator from TimeLogic.

When the specificity strand and the stabilization strand of the surrogate antibody comprise nucleic acid sequences, the surrogate antibodies can be formed by placing the first and second strand in solution, heating the solution, and cooling the solution under conditions such that, upon cooling, the first and second strand anneal and form the antibody. Any hybridization that could occur between two first strands or two second strands would not be stable because of the significantly weaker affinity coefficients relative to the designed multi-nucleotide complementation bonds designed into each of the specificity regions and the corresponding constant domains.

D. Diverse Structures of Surrogate Antibodies

Surrogate antibodies are a class of molecules having a nucleic acid sequence arranged to form a stable binding cavity that provides specific ligand binding through conformational complementarity to the ligand, and affinity through cooperative hydrophobic, electrostatic, Van der Waals-forces, and/or hydrogen binding, except where the target/ligand is a nucleic acid composition and binding by means of Watson/Crick base pairing or triple helical association is desired. See, for example, Riordan et al. (1991) Nature 350:442-443. Accordingly, a diverse number of surrogate antibodies structures can be formed.

In one embodiment, the surrogate antibodies described herein can include one or more specificity strands having one or more than one specificity domains (loop structure), wherein each specificity domain is flanked by constant domains. Surrogate antibodies of the invention can therefore have 1, 2, 3, 4, 5 or more specificity domains. It is recognized that a surrogate antibody composed of at least one specificity strand having multiple specificity domains will require a stabilization strand having the corresponding stabilization domains that allow for the proper formation of the surrogate antibody. In addition, each of the specificity regions could be on separate strands, (distinct) strands or on the same strand and the specificity strand could be linear or circular. Furthermore, multiple spacer regions can also be found on either the specificity or stabilization stand.

In further embodiments, the antibodies can be formed using multiple oligonucleotides and thus dimers and/or trimers are can be used to form the final surrogate antibody structure. See, for example, FIGS. 2 and 3. Consequently, two or more intramolecular and/or intra-strand loops can be present in the molecule. Thus, in another embodiment, the surrogate antibody molecule comprises more than one oligonucleotide strand containing stabilization regions and constant regions that anneal to form a multimer with multiple binding loops/cavities.

The surrogate antibody molecule can include multiple specificity regions having a common size and nucleotide sequence or different sizes and nucleotide sequences to optimize surrogate antibody binding to ligands of varying sizes. The molecules can further comprise multiple spacer regions (S) with a common size and nucleotide sequence or spacer regions of different sizes and nucleotide sequences. The specificity regions can be present on separate oligonucleotide strands, and the surrogate antibody molecules can include multiple oligonucleotide strands with specificity regions that anneal to form multimers with multiple binding cavities.

Thus, in one embodiment, the surrogate antibody comprises a first and a second specificity strand and a stabilization strand, where the first specificity strand comprises a nucleic acid sequence having a first specificity region flanked by a first constant region and a second constant region; the second specificity strand comprises a nucleic acid sequence comprising a second specificity region flanked by a third and a fourth constant region. The stabilization strand comprises a first stabilization domain that interacts with said first constant region and a second stabilization domain that interacts with said second constant region and said stabilization strand further comprise a third stabilization domain that interacts with the third constant region and a fourth stabilization domain that interacts with the fourth constant region. In this embodiment, the stabilization strand, the first and/or the second specificity strand can comprise the same or distinct molecules. In yet other embodiments, the first and the second specificity strands can be identical or non-identical.

In another embodiment, the polyoligonucleotide surrogate antibody molecule comprises stabilization regions on juxtaposed oligonucleotide strands of from 2-100 complimenting nucleotides that link adjacent strands.

In another aspect, the invention relates to a polyoligonucleotide surrogate antibody molecule comprising adjacent, juxtaposed, oligonucleotides of different lengths, with stabilization regions composed of complimentary nucleotides that upon hybridization create one or more ligand-binding loops/cavities (i.e. specificity region).

In another embodiment, the polyoligonucleotide, surrogate antibody, molecule comprises a spacer region(s) having one or more nucleotides located on an oligonucleotide strand opposite and adjacent to the binding loop/cavity sequence of nucleotides on an opposing strand.

In another embodiment, the polyoligonucleotide, surrogate antibody, molecule comprises a spacer region nucleotide, or nucleotide sequence, that minimizes or eliminates stress in the molecule and modifies the size and/or conformation of the binding loop/cavity on the opposing oligonucleotide strand.

In another embodiment, the polyoligonucleotide, surrogate antibody, molecule comprises a stabilization region composed of 2 to 100 nucleotides that are complimentary to the nucleotides on an adjacent, juxtaposed, oligonucleotide strand.

In another embodiment, the polyoligonucleotide, surrogate antibody, molecule comprises a specificity region that comprises 3 to 100 nucleotides.

In another embodiment, the polyoligonucleotide, surrogate antibody molecule comprises a spacer region that comprises 0 to 100 nucleotides or alternatively, the spacer could comprise a molecule such as a phosphate moiety.

In another embodiment, the polyoligonucleotide, surrogate antibody molecule comprises multiple stabilization regions having a common nucleotide sequence and sequence length or different nucleotide sequence and sequence length.

In another embodiment, the polyoligonucleotide, surrogate antibody molecule comprises multiple specificity regions that have a common number of nucleotides and nucleotide sequence or different number of nucleotides and nucleotide sequence.

In another embodiment, the polyoligonucleotide, surrogate antibody ligand-binding surrogate antibody molecule comprises natural nucleotides, modified nucleotides, or a combination of natural and modified nucleotides.

In one embodiment, the polyoligonucleotide, surrogate antibody molecule, comprises one or more attached ligands that may be the same or different.

Accordingly, the surrogate antibody can be “multi-valent” and thereby contain multiple specificity domains contained on one or more specificity strands. Thus, the specificity domains of a multi-valent surrogate antibody (i.e., antibody loops) can be the same nucleotide sequence and of the same size. In other embodiments, the specificity domains (i.e., loops) can be different and thus form “pluri-specific” surrogate antibodies. The pluri-specific antibody will bind different ligands or different regions/epitopes of the same ligand. Accordingly, each specificity domain can be designed to bind the same target/ligand or different targets/ligands. In this way, a surrogate antibody can simultaneously bind two common determinates on a single cell, bind different determinants, or be able to bind a compound in two distinct orientations. For example, an antibody can bind a particular receptor in a preferred binding site and also in an allosteric position. Alternatively, the surrogate antibody can bind a particular pair of receptors on a given cell surface thereby increasing affinity through cooperative binding interactions or form a bridge between molecules or cells.

In another aspect, the invention relates to a polyoligonucleotide, surrogate antibody, ligand-binding molecule produced to any ligand of sufficient size to be retained by a filter or fractionated based upon size, charge, hydrophobicity, electrophylic mobility, unique label, etc.

The surrogate antibodies can further contain hinge regions (or spacer regions) between the separate loop structures. The surrogate antibodies can include a “hinge unit” or spacer that functions in a similar manner as hinge units in conventional antibodies. Spacers and/or hybridization sequences can be present between the structures on the specificity strand and/or between the stabilization domains of the stabilization strand to sterically optimize binding to adjacent targets, for example, a plurality of binding sites on adjacent cells or on a single cell. In this way, the spacer region can be used to eliminate bond stress in molecules, provide diversity to the size and shape of the binding cavity, alter specificity loop orientation, optimize agglutination or flocculation, or optimize energy (Fluor) transfer reactions. Accordingly, the surrogate antibody molecule can comprises multiple spacer regions having a common number of nucleotides and nucleotide sequence or different number of nucleotides and nucleotide sequence.

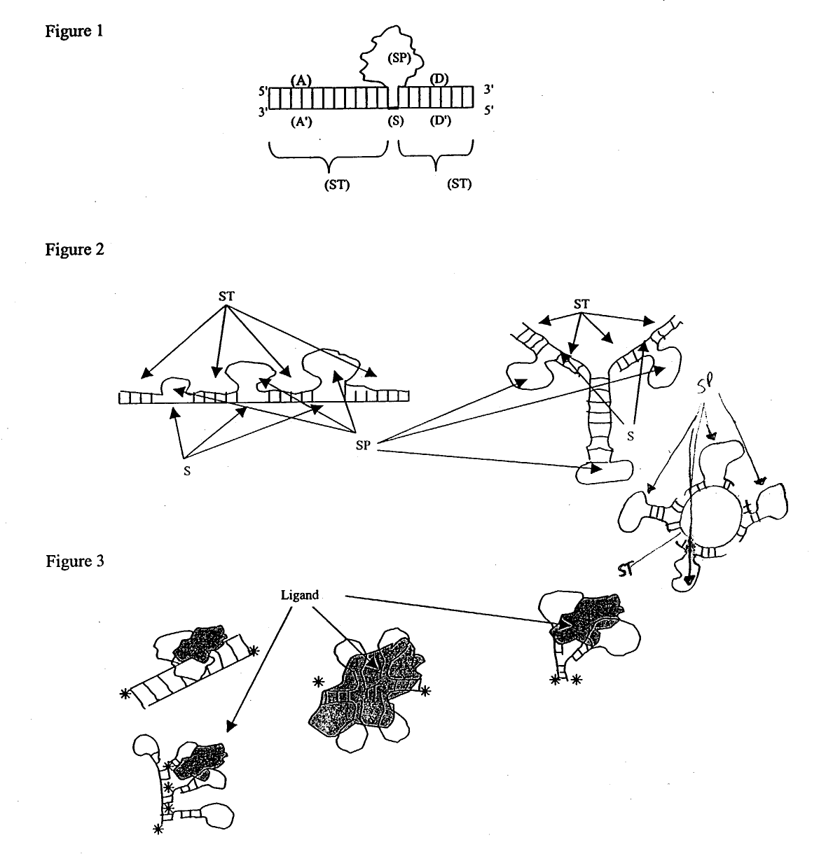

A representation of this type of molecule is shown in FIG. 1. FIG. 2 shows two embodiments of surrogate antibody molecules that include multiple specificity regions. In one embodiment, the surrogate antibody molecules include multiple specificity regions (SP), stabilization regions (ST) and spacer Regions (S) that collectively provide multi-dimensional ligand binding. These types of molecules are shown, for example, in FIGS. 3a-3d. As discussed above, in one embodiment, the surrogate antibody molecules can include stabilization regions and constant regions composed of opposing strands of complimentary nucleotides with cooperative interactions that collectively ensure adhesion of the strands and the stability and shape of the surrogate antibody molecule and the binding cavity. The surrogate antibody molecules can include a stabilization region (ST) composed of strands that contain a sequence of between 2 and 100 nucleotides, specificity regions (SPs) that contain between 3 and 100 nucleotides, and spacer regions (S) of the contain between 0 and 100 nucleotides. The surrogate antibody molecules can include multiple stabilization regions (ST) of a common size and nucleotide sequence or different sizes and nucleotide sequences.

It is further recognized that when the stabilization strand and the specificity strand comprise a nucleotide sequence, the strands can be contained on the same or distinct, (i.e., different) nucleic acid molecules. Thus, in another embodiment, the surrogate antibodies are formed from a single strand of nucleotides comprising a) a first constant region, a random nucleotide sequence loop-forming specificity region, a second constant region, a first spacer region, a second stabilization region that is capable of hybridizing to the second constant region, a second spacer region, and a first stabilization region that is capable of hybridizing to the first constant region. In one embodiment, each region contains between about one to about twenty nucleotides. The strand of nucleotides can be linear or cyclic, so long as when the stabilization regions and the constant regions are hybridized together with the non-hybridized specificity region forms a loop structure.

Alternatively, the specificity strands and stabilization strands need not be linked by a covalent interaction. Instead, the specificity strands and stabilization strands can comprise distinct molecules that interact (directly or indirectly) via non-covalent interactions. In this manner, when the specificity strand and the stabilization strand comprise nucleic acid sequences, each “distinct” strand will comprises a nucleic acid sequence having a 3′ and 5′ termini. Accordingly, the invention relates to a ligand-binding surrogate antibody molecule comprising an assembly of two or more single stranded RNA oligonucleotide strands, two or more single stranded DNA oligonucleotide strands, two or more TNA oligonucleotide strands, or a combination of two or more single stranded RNA, DNA, or TNA strands.

In other embodiments, the surrogate antibody molecules comprise double stranded DNA composed of two juxtaposed single stranded DNA molecules, multiple oligonucleotides hybridized to a complimenting longer oligonucleotide so that the multiple oligonucleotides each forms a binding cavity resulting in a molecule capable of simultaneous and multiple ligand binding, or juxtaposed chains of oligonucleotides that produce a stable molecule having one or more ligand binding sites.

The nucleotides used to prepare the surrogate antibodies (i.e., the specificity strand and, in some embodiments, the stabilization strand) can be naturally occurring or modified. Such modifications include alterations in the components of the specificity strand or the stabilization strand that results in the attachment of a “functional moiety”. As discussed in further detail below, the moiety can be attached via covalent or non-covalent interactions. Examples of these modifications in the surrogate antibody molecule include nucleotides that have been modified with amines, diols, thiols, phosphorothioate, glycols, fluorine, hydroxyl, fluorescent compounds (e.g. FITC), avidin, biotin, aromatic compounds, alkanes, and halogens. Such modifications can further include, but are not limited to, modifications at cytosine exocyclic amines, substitution of 5-bromo-uracil (Golden et al. (2000) J. of Biotechnology 81:167-178), backbone modifications, methylations, unusual base-pairing combinations and the like. See, for a review, Jayasena et al. (1999) Clinical Chemistry 45:1628-1650.

Those of skill in the art are aware of numerous modifications to nucleotides and to phosphate linkages between adjacent nucleotides that render them more stable to exonucleases and endonucleases (Uhlmann et al. (1990) Chem Rev. 90:543-98 and Agraul et al. (1996) Trends Biotechnology 14:147-9 and Usman et al. (2000) The Journal of Clinical Investigations 106:1197-1202). Such functional moieties include, for example, modifications at the 2′ position of the sugars (Hobbs et al. (1973) Biochemistry 12:5138-45 and Pieken et al. (1991) Science 253:314-7). For instance, the modified nucleotide could be substituted with amino and fluoro functional groups at the 2′ position. In addition, further functional moieties of interest include, 2′-O-methyl purine nucleotides and phosphorothioate modified nucleotides (Green et al (1995) Chem. Biol. 2:683-695; Vester et al. (2002) J. Am. Chem. Soc. 124:13682-13683; Rhodes et al. (2000) J. Biol. Chem. 37:28555-28561; and, Seyler et al. (1996) Biol. Chem. 377:67-70). Accordingly, in another embodiment, the surrogate antibody molecules comprise functional moieties comprising modified nucleotides that stabilize the molecule in the presence of serum nucleases.

Other functional moieties of interest include chemical modifications to one or more nucleotides in the specificity domain of the specificity strand, wherein the modified nucleotide introduces hydrophobic binding capabilities into the specificity domain. In certain embodiments, this chemical modification occurs at the 2′ position of the nucleotide sugar, nitrogenous base, or phosphate molecule. Such modifications are known in the art and include for example, non-polar, non-hydrogen binding shape mimics such as 6-methyl purine and 2,4-difluorotolune (Schweizer et al. (1995) J Am Chem Soc 117:1863-72 and Guckian et al. (1998) Nat Struct Biol 5:950-9, both of which are herein incorporated by reference). Additional modifications include imizadole, phenyl, proline, and isoleucyl.

In other embodiments, it is desirable to preferentially amplify the specificity strand of the surrogate antibody molecule. By “preferentially amplify” is intended that the specificity strand of the surrogate antibody molecule is amplified during the amplification step at an elevated frequency as compared to the amplification level of the corresponding stabilization strand. As such, an additional functional moiety of interest comprises a modification that allows for the preferential amplification of the specificity strand of the surrogate antibody molecule. While methods of amplifying the surrogate antibodies are discussed in further detail elsewhere herein, the type of modification that would allow this type of amplification are known in the art, and include, for example, a modification to at least one nucleotide on the stabilization strand that increases resistance to polymerase activity in a PCR reaction. Such modifications include any functional moiety that disrupts amplification including, for example, biotin.

Additional functional moieties of interest include, for example, a reporter molecule. As used herein a “reporter molecule” refers to a molecule that permits the detection of the surrogate antibody that it is attached to. Accordingly, in another embodiment, the incorporation or attachment of a “reporter” molecule as a functional moiety permits detection of the surrogate antibody and the complexed target ligand. Such reporter molecules include, for example, a polypeptide; radionucleotides (e.g. ³²P); fluorescent molecules (Jhaveri et al. (2000) J. Am. Chem. Soc. 122:2469-2473, luminescent molecules, and chromophores (such as FITC, Fluorescein, TRITC, Methyl Umbiliferone, luminol, luciferin, and Texas Red (Sumedha et al. (1999) Clinical Chemistry 45:1628-1649, Wilson et al. (1998) Clin Chemistry 44:86-91, and (2000) Nature Biotechnology 18:345-349); enzymes (e.g. Horseradish Peroxidase, Alkaline Phosphatase, Urease, β-Galactosidase, Peroxidase, proteases, etc.), lanthanide series elements (e.g. Europium, Terbium, Yttrium), and microspheres (e.g. sub-micron polystyrene, dyed or undyed) Such reporter molecules allow for direct qualitative or quantitative detection, or energy transfer reactions.

In one embodiment, the functional moiety comprising a reporter molecule is digoxigenin. Detection of this functional moiety is achieved by incubation with anti-digoxigenin antibodies coupled directly to several different fluorochromes or enzymes or by indirect immunofluorescence. See, Ausubel et al. Current Protocols in Molecular Biology, John Wiley & Sons, Inc. and Celeda et al. (1992) Biotechniques 12:98-102, both of which are herein incorporated by reference. Additional molecules that can act as reporters include biotin and polyA tails.

In another embodiment, the surrogate antibody molecules having multiple reporter molecules can be used in a test method to amplify the sensitivity of a test method.

In another embodiment, the functional moiety is an affinity tag (i.e., “binding molecule”) that can be used to attach surrogate antibodies to a solid support or to other molecules in solution. Thus, the isolation of the ligand-bound surrogate antibody complexes can be facilitated through the use of affinity tags coupled to the surrogate antibody. As used herein, an affinity tag is any compound that can be associated with a surrogate antibody molecule and which can be used to separate compounds or complexes and/or can be used to attach compounds to the surrogate antibody. Preferably, an affinity tag is a compound, such as a ligand or hapten that binds to or interacts with another compound, such as a ligand-binding molecule or an antibody. It is also preferred that such interactions between the affinity tag and the capturing component be a specific interaction, such as between a hapten and an antibody or a ligand and a ligand-binding molecule. For example, when attaching surrogate antibody molecules to a column, microplate well, or tube containing immobilized streptavidin, surrogate antibody molecules prepared using biotinylated primers result in their binding to the streptavidin bound to the solid phase. Other affinity tags used in this manner can include a polyA sequence, protein A, receptors, antibody molecules, chelating agents, nucleotide sequences recognized by anti-sense sequences, cyclodextrin and lectins. Additional affinity tags, described in the context of nucleic acid probes, have been described by Syvanen et al. (1986) Nucleic Acids Res. 14:5037. Preferred affinity tags include biotin, which can be incorporated into nucleic acid sequences (Langer et al. (1981) Proc. Natl. Acad Sci. USA 78:6633) and captured using streptavadin or biotin-specific antibodies. A preferred hapten for use as an affinity tag is digoxygenin (Kerkhof (1992) Anal Biochem. 205:359-364). Many compounds for which a specific antibody is known or for which a specific antibody can be generated can be used as affinity tags. Such affinity tags can be captured by antibodies that recognize the compound. Antibodies useful as affinity tags can be obtained commercially or produced using well established methods. For example, Johnston et al. (1987) Immunochemistry In Practice (Blackwell Scientific Publications, Oxford, England) 30-85, describe general methods useful for producing both polyclonal and monoclonal antibodies.

Other affinity tags are anti-antibody antibodies. Such anti-antibody antibodies and their use are well known. For example, anti-antibody antibodies that are specific for antibodies of a certain class or isotype or sub-class (for example, IgG, IgM), or antibodies of a certain species (for example, anti-rabbit antibodies) are commonly used to detect or bind other groups of antibodies. Thus, one can have an antibody to the affinity tag and then this antibody:affinity tag:synthetic activity complex can then be purified by binding to an antibody to the antibody portion of the complex.

Another affinity tag is one that can form selectable cleavable covalent bonds with other molecules of choice. For example, an affinity tag of this type is one that contains a sulfur atom. A nucleic acid molecule that is associated with this affinity tag can be purified by retention on a thiopropyl sepharose column. Extensive washing of the column removes unwanted molecules and reduction with β-mercaptoethanol, for example, allows the desired molecules to be collected after purification under relatively gentle conditions.

In yet other embodiments, the functional moiety is incorporated into the specificity strand to expand the genetic code. Such moieties include, for example, IsoG/IsoC pairs and 2,6-diaminopyrimide/xanthine base pairs (Piccirilli et al. (1990) Nature 343:537-9 and Tor et al. (1993) J Am Chem Soc 115:4461-7); methyliso C and (6-aminohexyl)isoG base pairs (Latham et al. (1994) Nucleic Acid Research 22:2817-22), benzoyl groups (Dewey et al. (1995) J Am Chem Soc 117:8474-5 and Eaton et al. (1997) Curr Opin Chem Biol 1:10-6) and amino acid side chains.

Other functional moieties of interest include a linking molecule (i.e., iodine or bromide for either photo or chemical crosslinking; a —SH for chemical crosslinking); a therapeutic agent (i.e., compounds used in the treatment of cancer, arthritis, septicemia, myocardial arrhythmia's and infarctions, viral and bacterial infections, autoimmune and prion diseases); a chemical modification that alters biodistribution, pharmacokinetics and tissue penetration, or any combination thereof. Such modifications can be at the C-5 position of the pyrimidine residues.

Functional moieties incorporated into the surrogate antibody (either in the stabilization strand or the specificity strand or both) may be multi-functional (i.e., the moiety could allow for labeling and affinity delivery, nuclease stabilization and/or produce the desired multi-therapeutic or toxicity effects. These various “functional moiety” modification find use, for example, in aiding detection for applications such as fluorescence-activated cell sorting (Charlton et al. (1997) Biochemistry 36: 3018-3026 and Davis et al. (1996) Nucleic Acid Research 24:702-703), enzyme linked oligonucleotide assays (Drolet et al. (1996) Nat. Biotech 14:1021-1025), and other diagnostic assays, some of which are discussed elsewhere herein. In addition, conjugation with a technetium-99 m chelatin cage would enable in vivo imaging. See, for example, Hnatowich et al. (1998) Nucl. Med. 39:56-64.

In addition, aptamers known to bind, for example, cellulose (Yang et al. (1998) Proc. Natl. Acad. Sci. 95: 5462-5467) or Sephadex (Srisawat et al. (2001) Nucleic Acid Research 29) have been identified. These aptamers could be attached to the surrogate antibody and used as a means to isolate or detect the surrogate antibody molecules. Additional functional moieties of interest include the addition of polyethylene glycerol to decrease plasma clearance in vivo (Tucker et al. (1999) J. Chromatography 732:203-212 or the addition of a diacylglycerol lipid group (Willis et al. (1998) Bioconjugate Chem. 9:573-582). In addition, the functional moiety having anti-microbial activity (i.e., anti-bacterial, anti-viral, or anti-fungal) properties could be used with the surrogate antibody as an anti-bioterror agent to overwhelm possible modifications of pathogenic organisms and viruses. As discussed in further detail elsewhere herein, the attachment of functional moieties find use in various methods.

Various methods for attaching the functional moiety to the surrogate antibody structure are known in the art. For example, bioconjugation reactions that provide for the conjugation of polypeptides or various other compounds of interest to the surrogate antibody can be found, for example, in Aslam et al. (1999) Protein Coupling Techniques for Biomed Sciences, Macmillan Press and Solulink Bioconjugation systems at www.solulink.com

A functional moiety can be attached to any region of the specificity stand or the stabilization strand or any combination thereof. In one embodiment, the functional moiety is attached to one or more of the constant domains and/or stabilization domains. In other embodiments, the functional moiety is attached to the specificity domain. One of skill in the art will recognized that site of attachment of the functional moiety will depend on the desired functional moiety.

Additional functional moieties include various agents that one desires to be directed to the location of the target ligand. The agent for delivery can be any molecule of interest, including, a therapeutic agent or a drug delivery vehicle. Such agents and their method of deliveries are disclosed elsewhere herein.

The functional moiety(ies) chosen to incorporate into the surrogate antibody structure can be selected depending on the environmental conditions in which the surrogate antibody will be contacted with its ligand or potential ligand. For example, generating surrogate antibody libraries containing molecules having ionizable groups may provide surrogate antibodies that are sensitive to salt, and the presence of metal chelating groups may lead to surrogate antibodies that are sensitive to specific metal ions. See, for example, Lin et al. (1994) Nucleic Acids Res 22:5229-34 and Lin et al. (1995) Proc Natl Acad Sci USA 92:11044-8.

In any of the various methods and compositions described herein, various functional moieties can be conjugated onto one or more strands that form the antibodies, in one or more positions on the strands. The strands can be covalently linked to one or more, or three or more, different types of moieties.

The surrogate antibodies can be configured to contain juxtaposed oligonucleotide strands that provide multiple sites for the attachment of auxiliary molecules to the specificity or stabilization strands. For example, when the specificity strand and the stabilization strand comprise nucleic acid sequences, the auxiliary molecules can be attached to the 3′ and/or 5′ end.

In another embodiment, the polyoligonucleotide, surrogate antibody molecule comprises one or more ligands affixed using modified primers that are specific for each of the constituent oligonucleotides of the surrogate antibody molecule.

In another aspect, the invention relates to a method of attaching one or more ligands in a directed fashion to the oligonucleotides of a surrogate antibody molecule using modified primers that target a unique oligonucleotide sequence on one or more of the constituent oligonucleotide strands.

One advantage of nucleic acid-based surrogate antibodies over natural antibodies is their ability to be readily assembled in vitro, using PCR amplification plus assembly by annealing of oligonucleotides that do not contain specificity regions. Another advantage is the ability to produce antibody molecules without the need to use animals, or animal facilities. They also eliminate the need to maintain viable tissue cultures during the selection process, allowing the capture and amplification of surrogate antibody molecules to occur directly in a sample matrix. This minimizes the issue of sample matrix compatibility and reduces the time to produce compatible and effective reagents. Surrogate antibody molecules eliminate the need to stimulate and mature an immune response. Another advantage is the simplicity of labeling surrogate antibody molecules using modified primer molecules or modified nucleotides. Another advantage is their small, hypoimmunogenic primary structure with enhanced mobility.

II. Surrogate Antibody Libraries

Compositions of the invention further comprise populations of surrogate antibodies. By “population” is intended a group or collection that comprises two or more (i.e., 10, 100, 1,000, 10,000, 1×10⁶, 1×10⁷, or 1×10⁸ or greater) surrogate antibodies. Various “populations” of surrogate antibodies are provided, including, for example, a library of surrogate antibodies, which as discussed in more detail below, comprises a population of surrogate antibodies having a randomized specificity region. The various populations of surrogate antibodies can be found in a mixture or in a substrate/array.

As provided elsewhere herein, the library of surrogate antibodies progresses through a series of iterative in vitro selection techniques that allow for the identification/capture of the desired surrogate antibody(ies). Each round of selection produces a selected population of surrogate antibody molecules that have an increased specificity and/or binding affinity to the desired ligand as compared to the library. Such populations of selected surrogate antibodies are discussed in more detail below.

In one embodiment, the population of surrogate antibodies comprises a library. A library of surrogate antibody molecules is a mixture of stable, pre-formed, surrogate antibody molecules of differing sequences, from which antibody molecules able to bind a desired ligand are captured. As used herein, a library of surrogate antibody molecules comprises a population of molecules comprising a specificity strand and a stabilization strand. The specificity strand comprises a nucleic acid sequence having a specificity region flanked by a first constant region and a second constant region; and, the stabilization strand comprises a first stabilization domain that interacts with said first constant region and a second stabilization domain that interacts with said second constant region. In addition, each of the first constant regions of the specificity strands in the population are identical; each of the second constant regions of the specificity strands in the population are identical; each of the specificity region of the specificity strands in said population are randomized; and, each of the stabilization strands in said population are identical. It is recognized that a library of surrogate antibody molecules having any of the diverse structures, described elsewhere herein, can be assembled.

A library of surrogate antibody molecules can be prepared that includes one or more members that have a binding cavity that permits attachment to a target ligand through hydrophobic, hydrogen, electrostatic, and Van der Waals bonding interactions in a manner similar to the ligand bonding mechanism observed in a native antibody molecule. The library can include molecules that obtain their structural stability from juxtaposed chains of complimentary nucleotide residues, each residue pair joined by covalent or non-covalent (e.g., Watson-Crick pairing) interactions so that the cumulative binding force of the juxtaposed chains prevents their separation. The library can include surrogate antibody molecules composed of paired strands of nucleic acids (e.g. DNA) such that one nucleic acid strand contains a greater number of nucleotide residues than the other and forms a stable loop structure.

As such, the constant regions on either side of the specificity region not only provide stabilization by binding with the stabilization regions of the stabilization strand, but can also be used to facilitate the amplification of the surrogate antibodies and the attachment of multiple molecules that can include reporter molecules and therapeutic agents. The library of surrogate antibodies includes a plurality of the surrogate antibodies, where the plurality of surrogate antibodies includes a plurality of different loop structures. The plurality of loop structures in the library allows the capture and identification of surrogate antibodies having the proper loop structure, from the plurality of loop structures that function as antibodies that bind to a particular antigen.

As used herein, a library typically includes a population having between ˜2 and 1×10¹⁴ surrogate antibodies. Alternatively, the surrogate antibody library used for selection can include a mixture of between about 2 and 10¹⁸, between 10⁹ and 10¹⁴, between about 10⁹ and 10¹⁹, between about 10⁹ and 10²⁴, between about 2 and 10²⁷ or more surrogate antibodies having a contiguous randomized sequence of at least 10 nucleotides in length in each binding cavity (i.e., specificity domain). In yet other embodiments, the library will comprise at least 3, 10, 100, 1000, 10000, 1×10⁵, or 1×10⁶, 1×10⁷, 1×10¹⁰, 1×10¹⁴, 1×10¹⁸, 1×10²², 1×10²⁵, 1×10²⁷ or greater surrogate antibody molecules having a randomized or semi-random specificity domain. The molecules contained in the library can be found together in a mixture or in an array.

The library can include surrogate antibodies formed from naturally-occurring nucleic acids or fragments thereof, chemically synthesized nucleic acids, enzymatically synthesized nucleic acids or nucleic acids made by combinations thereof. Such nucleotide modifications have been discussed in more detail elsewhere herein.

In certain other instances of usage herein, the term “population” may be used to refer to polyclonal or monoclonal surrogate antibody preparations of the invention having one or more selected characteristics.

A polyclonal surrogate antibody library or “population of polyclonal antibodies” comprises a population of individual clones of surrogate antibodies assembled to produce polyclonal libraries with enhanced binding to a target ligand. Once a surrogate antibody, or a plurality of separate surrogate antibody clones, are found to meet target performance criteria they can be assembled into polyclonal reagents that provide multiple epitope recognition and greater sensitivity/avidity in detecting the target ligand. It is recognized that a population of polyclonal surrogate antibodies can represent a pool of molecules obtained following the capture and amplification steps to a desired ligand. Alternatively, a population of polyclonal surrogate antibodies could be formed by mixing at least two individual monoclonal surrogate antibody clones having the desired ligand binding characteristics.

Virtually any substance introduced into a vertebrate, but not all substances in all vertebrates, can elicit an antibody response. The antibody repertoire in humans consists of 1011 different antibody molecules representing approximately 2.5-3.5×10⁸ different binding specificities. The human genome contains multiple copies of the V, D, and J gene segments that are responsible for transcribing the amino acid sequence of the heavy and light chain variable regions of the antibody binding site. These genes in different combinations on the heavy and light chains account for the binding diversity of the molecule. The kappa (κ) light chain contains approximately 40 Vκ gene segments, 5 Jκ segments, accounting for potentially 200 permutations. The lambda (λ,) light chain contains approximately 30 Vλ, and 4 Jκ or 120 possible permutations. The heavy chain contains approximately 65 Vh gene segments, 27 Dh segments, and 6 Jh segments accounting for around 11,000 combinations. Pairing of the two chains to form the binding cavity provides 320×11,000, or 3.5×10⁶, combinations or binding specificities.

In reality, the extent of binding diversity is less than this theoretical calculation because all V region segments are not expressed in the same frequency, some are common in all antibodies, and others are rarely found. Some Vh and V1 sequences pair poorly together.

Offsetting these limitations there exists additional diversity provided by imprecise joining of V, J, and D regions gene segments and somatic hypermutation that introduces point mutations into rearranged heavy and light chain genes at a high rate giving rise to mutant immunoglobulin gene products.

The binding diversity of surrogate antibody molecules is not limited by the diversity of gene segments within the genome. The size of the binding cavity/loop and epitope dimensions are not constrained by evolution. The binding repertoire of surrogate antibody is a function of the constrained conformation and the number of different nucleotide bases, functional moieties, and number of nucleotide residues that are used in the specificity region of the molecule. A library having a specificity region composed of 40 natural nucleotides potentially has 1.2×10²⁴ specificities. The selective use of modified bases in conjunction with natural bases again increases the diversity of the antibody repertoire.

A. Forming the Randomized Population of Specificity Regions

Methods of producing or forming a population of specificity strands having randomized specificity domains are known in the art. For example, the specificity region(s) can be prepared in a number of ways including, for example, the synthesis of randomized nucleic acid sequences and selection from randomly cleaved cellular nucleic acids. Alternatively, full or partial sequence randomization can be readily achieved by direct chemical synthesis of the nucleic acid (or portions thereof) or by synthesis of a template from which the nucleic acid (or portions thereof) can be prepared, by using appropriate enzymes. See, for example, Breaker et al. (1997) Science 261:1411-1418; Jaeger et al. (1997) Methods Enzy 183:281-306; Gold et al. (1995) Annu Rev Biochem 64:763-797; Perspetive Biosystems (1998) and Beaucage et al. (2000) Current Protocols in Nucleic Acid Chemistry John Wily & Sons, N.Y. 3.3.1-3.3.20; all of which are herein incorporated by reference. Alternatively, the oligonucleotides can be cleaved from natural sources (genomic DNA or cellular RNA preparations) and ligated between constant regions.

Randomized is a term used to describe a segment of a nucleic acid having, in principle, any possible sequence of nucleotides containing natural or modified bases over a given length. As discussed above, the specificity region can be of various lengths. Therefore, the randomized sequences in the surrogate antibody library can also be of various lengths, as desired, ranging from about ten to about 90 nucleotides or more. The chemical or enzymatic reactions by which random sequence segments are made may not yield mathematically random sequences due to unknown biases or nucleotide preferences that may exist. The term “randomized” or “random,” as used herein, reflects the possibility of such deviations from non-ideality. In the techniques presently known, for example sequential chemical synthesis, large deviations are not known to occur. For short segments of 20 nucleotides or less, any minor bias that might exist would have negligible consequences. The longer the sequences of a single synthesis, the greater the effect of any bias.

In addition, a bias can be deliberately introduced into randomized sequence, for example, by altering the molar ratios of precursor nucleoside (or deoxynucleoside) triphosphates of the synthesis reaction. A deliberate bias may be desired, for example, to approximate the proportions of individual bases in a given organism, or to affect secondary structure. See, Hermes et al. (1998) Gene 84:143-151 and Bartel et al. (1991) Cell 67:529-536, both of which are herein incorporated by reference. See also, Davis et al. (2002) Proc. Natl. Acad. Sci. 99:11616-11621, which generated a randomized population having a bias comprising a specified stem loop structure. Thus, as used herein, a randomized population of specificity domains may be generated to contain a desirable bias in the primary sequence and/or secondary structure of the domain.

It is not necessary (or possible from long randomized segments) that the library includes all possible variant sequences. The library can include as large a number of possible sequence variants as is practical for selection, to insure that a maximum number of potential binding sequences are identified. For example, if the randomized sequence in the specificity region includes 30 nucleotides, it would contain approximately 10¹⁸ (i.e. 430) sequence permutations using the 4 naturally occurring bases.

Practical considerations include the number of templates on DNA synthesis columns, and the solubility of the surrogate antibodies and the targets in solution. While there is no theoretical limit for the number of sequences in the surrogate antibody library, libraries that include randomized segments containing an excessive number of bases can be inconvenient to produce. It is not necessary for the library to include all possible sequences to select an appropriate surrogate antibody.

The size of the loop structure (specificity region) of individual members within the library can be substantially the same or different. Iterative libraries can be used, where the loop structure varies in size in each library or are combined to form a library of mixed loop sizes, for the purpose of identifying the optimum loop size for a particular target ligand.

As discussed above, the specificity strand may contain various functional moieties. Methods of forming the randomized population of specificity strands will vary depending on the functional moieties that are to be contained on the strand. For example, in one embodiment, the functional moieties comprise modified adenosine residue. In this instance, the specificity strand could be designed to contain adenosine residues only in the specificity domain. The nucleotide mixture used upon amplification will contain the adenosine having the desired functional moieties (i.e., moieties that increase hydrophobic binding characteristics). In other instances, the functional moiety can be attached to the surrogate antibody following the synthesis reaction.

B. Generating a Surrogate Antibody library

Once the population of specificity strands having a randomized assortment of specificity regions has been formed, the surrogate antibodies can be formed (as discussed elsewhere herein) by contacting the specificity strand with an appropriate stabilization strand under the desired conditions.

Methods are provided for generating a library of surrogate antibody molecule. The method comprises: a) providing a population of specificity strands wherein i) the population of specificity strands is characterized as a population of nucleic acid molecules; ii) each of the specificity strands in said population comprises a nucleic acid sequence having a specificity region flanked by a first constant region and a second constant region; iii) each of the first constant region of the specificity strands in the population are identical; iv) each of the second constant region of the specificity strands in said population are identical; and, v) each of the specificity regions of said specificity strands in said population are randomized. The population of specificity strands is contacted with a stabilization strand; wherein the stabilization strand comprises a first stabilization domain that interacts with said first constant region and a second stabilization domain that interacts with said second constant region, wherein said contacting occurs under conditions that allow for the first stabilization domain to interact with the first constant region and the second stabilization domain to interacts with the second constant region. Also provided are surrogate antibody libraries produced by this method. In other embodiments surrogate antibodies that compose the library have a specificity strand and a stabilization strand contained on distinct strands.

In one embodiment, a surrogate antibody library comprising a specificity strand and a stabilization strand comprising nucleic acid sequences can be prepared by hybridizing a long oligonucleotide strand containing a 5′ end complimenting nucleotide sequence, a random nucleotide intervening sequence, and a 3′ end complimenting sequence, to a short oligonucleotide strand containing two complimenting sequences at the 5′ and 3′ ends.

It is further recognized that it may be beneficial to produce a population of surrogate antibodies having a randomized specificity domain that varies in length. In this manner, the library could be used in a “multi-fit” process of surrogate antibody development that defines the optimal surrogate antibody cavity size to use for any given target. The process allows surrogate antibody binding to improve upon the binding characteristics of native antibody molecules where the size of the paratope (binding site) is finite for all ligands regardless of size. The “multi-fit” process identifies a cavity size with spatial characteristics that enhance the fit, specificity, and affinity of the surrogate antibody-ligand complex. The “multi-fit” process can identify as an ideal binding loop/cavity one that is not restricted in size or dimensionality by the precepts of evolution and genetics. As such surrogate antibody molecules challenge the conventional paradigm regarding the size of an epitope or determinant as shaped by the dependency of science and research on the properties of native antibody molecules. Preliminary “multi-fit” ligand capture rounds are performed using a heterogeneous population of surrogate antibodies containing cavities of varying size and conformation. The optimal cavity size for surrogate antibody library preparation is indicated by the sub-population having a cavity size that exhibits the highest degree of ligand binding after a limited number of capture and amplification cycles.

III. Kits

The disclosed surrogate antibody molecules and the various populations of such molecules (i.e., monoclonal surrogate antibodies, polyclonal surrogate antibodies, selected populations of antibody molecules, and libraries) can also be used as reagents in kits. For example, kits for the identification of a desired ligand are provided. The kit comprises a surrogate antibody population and suitable buffers to detected the desired ligand. In one example, the surrogate antibody and the buffer can be present in the form of solutions, suspensions, or solids such as powders or lyophilisates. The reagents can be present together, separated from one another, or on a suitable support. The disclosed kit can also be used as a diagnostic agent or to identify the function of unknown genes.

IV. Methods of Screening a Surrogate Antibody Library

As discussed above, the present invention provides methods and compositions for the formation of surrogate antibodies and libraries containing surrogate antibodies. Also provided are methods that allow the screening of a surrogate antibody library or a selected population of surrogate antibodies to identify or “capture” a surrogate antibody or a population of surrogate antibodies having the desired ligand-binding characteristics. In this manner, surrogate antibody molecules are selected for subsequent cloning from a library of pre-synthesized multi-stranded molecules that contain a random sequence ligand-binding cavity (specificity region), or cavities, and stabilization regions that stabilize the structure of the molecule in solution.

Generally, surrogate antibodies that bind to a particular target/ligand are captured from a starting surrogate antibody library by contacting one or more ligand with the library, binding one or more surrogate antibodies to the target(s)/ligand(s), separating the surrogate antibody bound ligand from unbound surrogate antibody, and identifying the bound target and/or the bound surrogate antibodies.

For example, in one embodiment, the present invention provides a method for screening a surrogate antibody library comprising:

a) contacting at least one ligand with a library of surrogate antibody molecules, said library comprising a population of surrogate antibody molecules comprising a specificity strand and a stabilization strand; wherein,

i) the specificity strand comprises a nucleic acid sequence having a specificity region flanked by a first constant region and a second constant region; and, the stabilization strand comprises a first stabilization domain that interacts with said first constant region and a second stabilization domain that interacts with said second constant region;

ii) each of the first constant regions of the specificity strands in the population are identical; each of the second constant region of the specificity strands in the population are identical; each of the specificity domains of the specificity strands in said population are randomized; and, each of the stabilization strands in said population are identical;

b) partitioning said ligand and said population of surrogate antibody molecules from said population of ligand-bound surrogate antibody complexes; and,

c) amplifying the specificity strand of the population of ligand-bound surrogate antibody complexes.

In still other embodiments, the method of screening a surrogate antibody library further comprises contacting said population of specificity strands of step (c) with a stabilization strand under conditions that allow for said first stabilization domain to interact with said first constant region and said second stabilization domain to interact with said second constant region.

In other embodiments, the stabilization strand and the specificity strand of the surrogate antibody molecules are distinct.

As discussed previously, the methods allow for the selection or capturing of a surrogate antibody molecule that interacts with the desired ligand of interest. The method thereby employs selection from a library of surrogate antibody molecules followed by step-wise repetition of selection and amplification to allow for the identification of the surrogate antibody molecule have the desired binding affinity and/or selectivity for the ligand of interest. As used herein a “selected population of surrogate antibody molecules” is intended a population of molecules that have undergone at least one round of ligand binding.

Accordingly, in another embodiment, the method of capturing a surrogate antibody comprises contacting a selected population of surrogate antibodies with the ligand of interest. In this embodiment, a library of molecules containing a randomized specificity domain need not be use, but rather a selected population of surrogate antibody molecules generated, for example, following the second, third, fourth, fifth, sixth, seventh or higher round of selection/amplification could be contacted with the desired ligand. In this embodiment, a method for capturing a surrogate antibody comprises:

a) contacting a ligand with a population of surrogate antibody molecules under conditions that permit formation of a population of ligand-bound surrogate antibody complexes, wherein said surrogate antibody molecule of the surrogate antibody population comprises a specificity strand and a stabilization strand,

said specificity strand comprising a nucleic acid sequence having a specificity region flanked by a first constant region and a second constant region; and,

said stabilization strand comprises a first stabilization domain that interacts with said first constant region and a second stabilization domain that interacts with said second constant region;

b) partitioning said ligand and said population of surrogate antibody molecules from said population of ligand-bound surrogate antibody complexes; and,

c) amplifying the specificity strand of said population of ligand-bound surrogate antibody complexes.

In other embodiments, the method of capturing a surrogate antibody molecule further comprises contacting said population of specificity strands of step (c) with a stabilization strand under conditions that allow for said first stabilization domain to interact with said first constant region and said second stabilization domain to interact with said second constant region. In yet other embodiments, the stabilization strand and the specificity strand are distinct.

Accordingly, in another embodiment, the process comprises preparing a ligand-binding surrogate antibody molecule(s) from a pre-assembled library of at least 2 surrogate molecules or, alternatively, 10⁹-10¹⁴ surrogate antibody molecules (0.17 nanomole-1.7 femptomole).

In another embodiment, the process comprises preparing a ligand-binding surrogate antibody reagent by capturing surrogate antibody from a pre-assembled library of surrogate antibody molecules having at least one specificity region composed of from 10 to 90 nucleotides, between 10 and 60 nucleotides, or between 10 and 40 nucleotides.

In another embodiment, the process comprises preparing a ligand-binding surrogate antibody reagent from a pre-assembled library of surrogate library molecules having specificity regions composed of a varying number and sequence of nucleotides or modified nucleotides that enhance ligand binding and/or stability.

In another embodiment, the process comprises preparing a ligand-binding surrogate antibody reagent to any molecule that is unable to penetrate a filter when complexed to a surrogate antibody.

In another embodiment, the process comprises preparing ligand-binding surrogate antibody molecules that involves separating surrogate antibody-ligand complexes in solution from uncomplexed surrogate antibody in the same solution.

In another embodiment, the process comprises preparing a ligand-binding surrogate antibody reagent using a filter that does not retain uncomplexed surrogate antibody molecules but does retain surrogate antibody molecules that are complexed to a target ligand.

In another embodiment, the process comprises preparing a ligand-binding surrogate antibody reagent, as above, using size-exclusion chromatography, size exclusion/molecular sieving filtration, affinity chromatography, ion-exchange chromatography, reverse phase chromatography, FACS or electrophoresis.

In another embodiment, the process comprises capturing surrogate antibody molecules from a surrogate antibody library of molecules having binding loops/cavities (specificity domains) with different dimensional configurations for the purpose of enhancing binding affinity and specificity to a target ligand.

In another embodiment, the process comprises producing a surrogate antibody having a binding loop/cavity (specificity domain) having a size and conformation that is determined by the number of nucleotides and nucleotide modifications, if any, that are used.

In another embodiment, the process comprises producing a surrogate antibody having a binding loop/cavity (specificity domain) not limited in size.

In another embodiment, the process comprises the simultaneous preparation of ligand-binding surrogate antibody molecules with different binding specificities.

In another embodiment, the process comprises the simultaneous preparation of ligand binding surrogate antibody molecules by incubating a single library of random binding surrogate antibody molecules with a library of target ligands able to be retained by a filter when bound to a surrogate antibody.

In another embodiment, surrogate antibodies can be assembled into libraries, which libraries can be used in high-throughput assays as described in more detail below.

In another aspect, the invention relates to a process for preparing a ligand-binding surrogate antibody reagent that captures ligand-binding surrogate molecule(s) present in a pre-assembled library of randomly binding surrogate antibody molecules.

A. Methods of Contacting:

By “contacting” is intended any method that allows a desired ligand of interest to interact with a surrogate antibody molecule or a population thereof. One of skill in the art will recognize that a variety of conditions could be used for this interaction. For example, the experimental conditions used to select surrogate antibodies that bind to various target ligands can be selected to mimic the environment that the target would be found in vivo or the anticipated in vitro application. Adjustable conditions that can be altered to more accurately reflect this binding environment include, but are not limited to, total ionic strength (osmolarity), pH, enzyme composition (e.g. nucleases), metalloproteins (e.g. hemoglobin, ceruloplasm) temperature and the presence of irrelevant compounds. Conditions that can be altered when developing surrogate antibody for in vitro environmental testing methods can include the aforementioned agents and conditions as well as solvents, surfactants, radionucleotides, normal constituents that may be present in soil, water, and air samples, volatile and semi-volatile compounds, inorganic and organic compounds. See, for example, Dang et al. (1996) J Mol Bio 264:268-278; O'Connell et al. (1996) Proc. Natl Acad Sci USA 93:5883-7; Bridonneu et al. (1999) Antisense Nucleic Acid Drug Dev 9: 1-11; Hicke et al (1996) J Clin Investig 98:2688-92; and, Lin et al. (1997) J Mol Biol 271:446-8, all of which are herein incorporated by reference.

Appropriate conditions to contact the ligand of interest and the surrogate antibody can be determined empirically based on the reaction chemistry. In general, the appropriate conditions will be sufficient to allow 1% to 5%, 5%-10%, 10% to 20%, 20% to 40%, 40% to 60%, 60% to 80%, 80% to 90%, or 90% to 100% % of the antibody molecule population to interact with the ligand.

B. Methods of Partitioning:

By “partitioning” is intended any process whereby surrogate antibody bound to target ligands, termed ligand-bound surrogate antibody complexes, are separated from surrogate antibodies not bound to target molecules. Partitioning can be accomplished by various methods known in the art. For example, surrogate antibodies bound to targets/ligands can be immobilized, or fail to pass through filters or molecular sieves, while unbound surrogate antibodies are not. Columns that specifically retain ligand-bound surrogate antibody can be used for partitioning. Liquid-liquid partition can also be used as well as filtration gel retardation, and density gradient centrifugation. The choice of the partitioning method will depend on properties of the target/ligand and on the ligand-bound surrogate antibody and can be made according to principles and properties known to those of ordinary skill in the art.

In one embodiment, partitioning comprises filtering a mixture comprising the ligand, the population of surrogate antibody molecules, and the population of ligand-bound surrogate antibody complexes through a filtering system wherein said filtering system is characterized as allowing for the retention of the ligand-bound surrogate antibody complex in the retentate and allowing the unbound surrogate antibodies to pass into the filtrate. Such filtering systems are known in the art. For example, various filtration membranes can be used. The term “filtration membrane” includes devices that separate on the basis of size (e.g. Amicon Microcon®, Pall Nanosep®), charge, hydrophobicity, chelation, and clathration.

The pore size used in the filtration process can be paired to the size of the target ligand and size of the surrogate antibody molecule used in the initial population of surrogate antibodies. For example, a cellular ligand having a 7-10 micron diameter will be retained by a membrane that excludes 7 microns. Surrogate antibody molecules having a 120 nucleotide bi-oligonucleotide structure when uncomplexed are easily eliminated as they pass through the membrane. Those bound to the ligand are captured in the retentate and used for assembly of the subsequent population. The preparation of a surrogate antibody to a BSA-hapten conjugate must use a pore that excludes the surrogate antibody-conjugate complex. A membrane that excludes 50,000 or 100,000 daltons effectively fractionates this surrogate antibody when bound to the conjugate from free surrogate antibody. Surrogate antibody prepared to a small protein, such as the enzyme Horseradish Peroxidase requires a membrane that would exclude molecules that are approximately 50,000 daltons or greater, while allowing the uncomplexed surrogate antibody to penetrate the filter. Target ligands can be chemically conjugated to larger carrier molecules or polymerized to enhance their size and membrane exclusion characteristics.

Alternative protocols used to separate surrogate antibodies bound to target ligands from unbound surrogate antibody[ies] are available to the art. For example, the separation of ligand-bound and free surrogate antibody molecules that exist in solution can be achieved using size exclusion column chromatography, reverse phase chromatography, size exclusion/molecular sieving filtering, affinity chromatography, electrophoretic methods, ion exchange chromatography, solubility modification (e.g. ammonium sulfate or methanol precipitation), immunoprecipitation, protein denaturation, FACS density gradient centrifugation. Ligand-bound and unbound surrogate antibody molecules can be separated using analytical methods such as HPLC and fluorescent activated cell sorters.

Affinity chromatography procedures using selective immobilization to a solid phase can be used to separate surrogate antibody bound to a target ligand from unbound surrogate antibody molecules. Such methods could include immobilization of the target ligand onto absorbents composed of agarose, dextran, polyacrylamide, glass, nylon, cellulose acetate, polypropylene, polyethylene, polystyrene, or silicone chips.

Method of amplifying the specificity strand of the surrogate antibody are described below, however, it is recognized that a surrogate antibody bound to the target ligand could be used in PCR amplification to produce oligonucleotide strand(s) having an integral specificity region(s) with or without separation from the affinity matrix.

A combination of solution and solid-phase separation could include binding a surrogate antibody to ligand conjugated microspheres that could be isolated based upon a physicochemical effect created by the surrogate antibody binding. Separate microsphere populations could individually be labeled with chromophores, fluorophores, magnetite conjugated to different target ligands or difference orientations of the same ligand. Surrogate antibody molecules bound to each microsphere population could be isolated on the basis of microsphere reporter molecule characteristic(s), allowing for production of multiple surrogate populations to different ligands simultaneously.

Accordingly, in another embodiment, the surrogate antibody molecules can bind any ligand, including, immunological haptens, organic environmental pollutants (e.g., polychlorinated biphenyls), therapeutic agents, substances of abuse, hormones, peptides, prions, nucleic acids and other molecules able to pass through a filter but that can be conjugated and retained by a filter.

Surrogate catalytic antibodies can be selected, based on binding affinity and the catalytic activity of the antibodies once bound. One way to select for catalytic antibodies is to search for surrogate antibodies that bind to transition state analogs of an enzyme catalyzed reaction.

In another embodiment, the surrogate antibody molecules can bind molecules that can be retained by a filter.

The methods can be used to simultaneously produce surrogate antibody molecules that bind to chemically multiple, chemically distinct, ligands. For example, the method can be used to select surrogate antibodies for a mixed population of target ligand conjugates unable to penetrate the membrane. Sequential incubation of a surrogate antibody population with un-conjugated filterable ligands allows for separation of non-specific surrogate antibody populations in the filtrate. Pre-incubation with filterable target ligands allows for rapid fractionation of SAb populations in the retenate for subsequent amplification.

C. Methods of Amplifying

Also provided are methods for amplifying the specificity strand of a surrogate antibody molecule, amplifying the specificity strands a population of surrogate antibodies, and/or amplifying the specificity strand(s) of a ligand-bound surrogate antibody complex. Amplifying or amplification means any process or combination of process steps that increases the amount or number of copies of a molecule or class of molecules. RNA molecules can be amplified by a sequence of three reactions: making cDNA copies of selected RNAs, using polymerase chain reaction to increase the copy number of each cDNA, and transcribing the cDNA copies to obtain RNA molecules having the same sequences as the selected RNAs. Any reaction or combination of reactions known in the art can be used as appropriate, including direct DNA replication, direct RNA amplification and the like, as will be recognized by those skilled in the art. The amplification method should result in the proportions of the amplified mixture being essentially representative of the proportions of different constituent sequences in the initial mixture.

In this manner, a population of specificity strands is generated. Thus, when the amplified specificity strands are contacted with the appropriate stabilization stand, a population of surrogate antibodies having the desired ligand binding affinity and/or specificity can be formed. Methods to selectively enhance the specificity of the ligand interaction and methods for enhancing the binding affinity of the population are provided below.

Once a desired surrogate antibody or set of surrogate antibodies is identified, it is often desirable to identify the nucleotide sequence of one or more of the monoclonal surrogate antibody clones and generate large amount of either a monoclonal or assembled polyclonal surrogate antibody reagent. In another embodiment, a monoclonal surrogate antibody can be generated (i.e., captured). In this embodiment, the method of capturing a surrogate antibody further comprises cloning at least one specificity strand from the population of amplified specificity strands. The cloned specificity strand can be amplified using routine methods and subsequently contacted with the appropriate stabilization strand under conditions that allow for said first stabilization domain to interact with said first constant region and said second stabilization domain to interact with said second constant region, and thereby producing a population of monoclonal surrogate antibodies.

Methods of amplifying nucleic acid sequences (such as those of the specificity strand) are known. Polymerase chain reaction (PCR) is an exemplary method for amplifying nucleic acids. PCR methods are described, for example in Saiki et al. (1985) Science 230:1350-1354; Saiki et al. (1986) Nature 324:163-166; Scharf et al. (1986) Science 233:1076-1078; Innis et al. (1988) Proc. Natl. Acad. Sci. 85:9436-9440; and in U.S. Pat. No. 4,683,195 and U.S. Pat. No. 4,683,202, the contents of each of which are incorporated herein in their entirety.

PCR amplification involves repeated cycles of replication of a desired single-stranded DNA (or cDNA copy of an RNA) employing specific oligonucleotide primers complementary to the 3′ and 5′ ends of the ssDNA, primer extension with a DNA polymerase, and DNA denaturation. Products generated by extension from one primer serve as templates for extension from the other primer. A related amplification method described in PCT published application WO 89/01050 requires the presence or introduction of a promoter sequence upstream of the sequence to be amplified, to give a double-stranded intermediate. Multiple RNA copies of the double-stranded promoter containing intermediate are then produced using RNA polymerase. The resultant RNA copies are treated with reverse transcriptase to produce additional double-stranded promoter containing intermediates that can then be subject to another round of amplification with RNA polymerase. Alternative methods of amplification include among others cloning of selected DNAs or cDNA copies of selected RNAs into an appropriate vector and introduction of that vector into a host organism where the vector and the cloned DNAs are replicated and thus amplified (Guatelli et al. (1990) Proc. Natl. Acad. Sci. 87:1874). In general, any means that will allow faithful, efficient amplification of selected nucleic acid sequences can be used. It is only necessary that the proportionate representation of sequences after amplification at least roughly reflects the relative proportions of sequences in the mixture before amplification. See, also, Crameri et al (1993) Nucleic Acid Research 21: 4110, herein incorporated by reference.

The method can optionally include appropriate nucleic acid purification steps.

Surrogate antibody strands that contain specificity region nucleotides will generally be capable of being amplified. Generally, any conserved regions used in this strand also will not include molecules that interfere with amplification. However, the invention can include the introduction of moieties, e.g. via selective chemistry, to the specificity regions or other regions that may interfere with amplification by methods such as PCR. Such surrogate antibodies can be produced by any necessary biological and/or chemical steps in accordance with the methods of the invention.

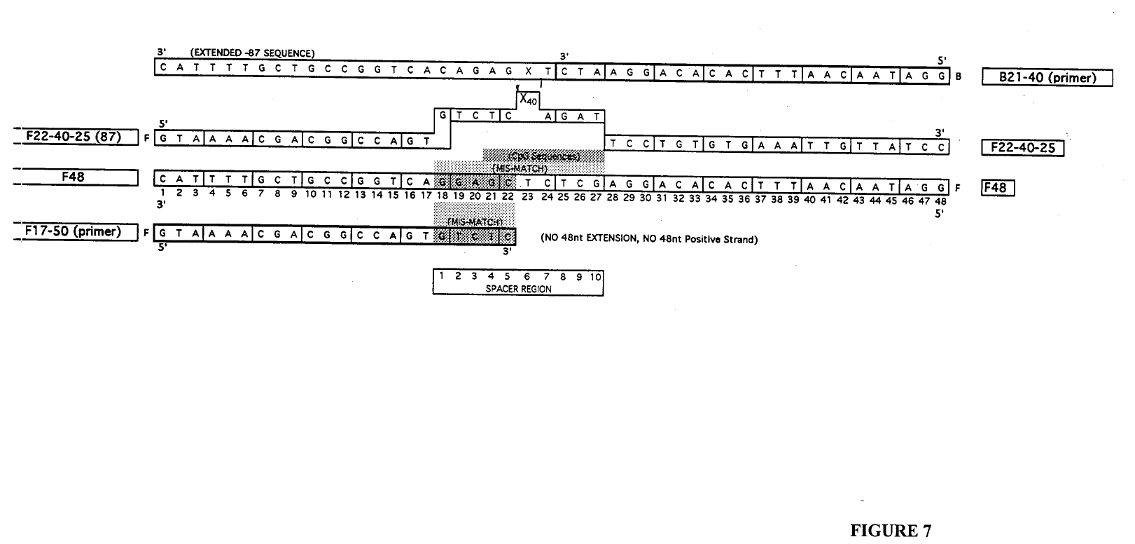

In other embodiments, the stabilization strand and the specificity strand contain a region of non-homology that can be used, in combination with the appropriate primers, to prevent the amplification of the stabilization strand. A non-limiting example of this embodiment appears in FIG. 7 and in Example 4 of the Experimental section. Briefly, in this non-limiting example, the stabilization strand and specificity strand lack homology in about 2, 3, 4, 5, 6, 8 or more nucleotides positioned 5′ to the specificity domain. See, shaded box in FIG. 7. The primer used to amplify the positive strand of the specificity strand is complementary to the sequences of the specificity strand. However, due to the mis-match design, this primer lacks homology at its 3′ end to the sequence of the stabilization strand. This lack of homology prevents amplification of the full-length negative stabilization strand. This method therefore allows for the preferential amplification of the specificity strand.

When the surrogate antibody comprises a stabilization strand and a specificity strand comprising a nucleic acid sequence, each of the strands (i.e., the juxtaposed surrogate antibody strands) that contain a linear array of stabilization sequence(s), constant regions, specificity sequence(s) and/or spacer sequence(s) is initially prepared by a DNA synthesizer. In one embodiment, the selection process for capturing and amplifying a specific, high affinity, surrogate antibody reagent preferentially amplifies only the strand(s) containing specificity region(s) sequence by PCR. As outlined above in more detail, the surrogate molecules are assembled by mixing these strands with the appropriate stabilization strands strand(s) that ensure proper alignment upon interaction of the constant and stabilization domains. Once the juxtaposed strands are mixed the solution is heated and the strands allowed to hybridize as the temperature is reduced. In other embodiments, the surrogate antibody may be formed without heating.

Thus, the present invention provides for a method of amplifying a surrogate antibody molecule comprising providing a specificity strand and a stabilization strand, said specificity strand comprising a nucleic acid sequence having a specificity region flanked by a first constant region and a second constant region; and, said stabilization strand comprises a first stabilization domain that interacts with said first constant region and a second stabilization domain that interacts with said second constant region; amplifying the specificity strand; and, contacting said specificity strands with said stabilization strand under conditions that allow for said first stabilization domain to interact with said first constant region and said second stabilization domain to interact with said second constant region. In some embodiment, the said stabilization strand and said specificity strand comprise distinct molecules.

D. Staging

The process of iterative selection of surrogate antibody elements that specifically bind to a selected target molecule with high affinity is herein designated “staging.” Staging is a term that implies the “capture and amplification” of surrogate antibody molecules that bind a target molecule/ligand that can be macromolecular or the size of an immunological hapten. The staging process can be modified in various ways to allow for this identification of the desired surrogate antibody. For instance, steps can be taken to allow for “specificity enhancement” and thereby eliminate or reduce the number of irrelevant or undesirable surrogate antibody molecules from the captured population. In addition, “affinity enhancement” can be performed and thereby allow for the selection of high affinity surrogate antibody molecules to the target ligand. The staging process is particularly useful in the rapid isolation and amplification of surrogate antibodies that have high affinity and specificity for the target molecule/ligand. See, for example, Crameri et al. (1993) Nucleic Acid Research 21:4410.

V. Method of Enhancing the Binding Specificity of a Surrogate Antibody or Population Thereof

Specific binding is a term that is defined on a case-by-case basis. In the context of a given interaction between a given surrogate antibody molecule and a given target, enhanced binding specificity results when the preferential binding interaction of a surrogate antibody with the target is greater than the interaction observed between the surrogate antibody and irrelevant and/or undesirable targets. The surrogate antibody molecules described herein can be selected to be as specific as required using the “staging” process to capture, isolate, and amplify specific molecules.

Accordingly, the present invention further provides a method of enhancing the binding specificity of a surrogate antibody comprising:

a) contacting a population of surrogate antibody molecules, said population of surrogate antibody molecules capable of binding a ligand of interest, with a non-specific moiety under conditions that permit formation of a population of non-specific moiety-bound surrogate antibody complexes,

wherein said surrogate antibody molecule of the surrogate antibody population comprises a specificity strand and a stabilization strand, said specificity strand comprising a nucleic acid sequence having a specificity region flanked by a first constant region and a second constant region; and, said stabilization strand comprises a first stabilization domain that interacts with said first constant region and a second stabilization domain that interacts with said second constant region;

b) partitioning said non-specific moiety and said population of non-specific moiety-bound surrogate antibody complexes from said population of surrogate unbound antibody molecules; and,

c) amplifying at least one of the specificity strand of said population of unbound surrogate antibody complexes of step (b).

In further embodiments, the method of enhancing the binding affinity further comprises contacting the population of specificity strands of step (c) above with a stabilization strand under conditions that allow for said first stabilization domain to interact with said first constant region and said second stabilization domain to interact with said second constant region.

In further embodiments, the population of surrogate antibodies comprises a library of surrogate antibodies and/or a population of selected antibodies. In other embodiment, the stabilization strand and the specificity strand comprise distinct molecules.

In this embodiment, the binding specificity of the surrogate antibody population is enhanced by contacting the population of surrogate antibodies with a non-specific moiety under conditions that permit formation of a population of non-specific moiety-bound surrogate antibody complexes. In this manner, surrogate antibodies that interact with both the target ligand and a variety of non-specific moieties can partitioned from the population of surrogate antibodies having a higher level of specificity to the desired ligand.

By “non-specific moiety” is intended any molecule, cell, organism, virus, chemical compound, nucleotide, or polypeptide that is not the desired target ligand. Depending on the desired surrogate antibody population being produced, one of skill in the art will recognize the most appropriate non-specific moiety to be used. For example, if the desired target is protein X which has 95% sequence identity to protein Y, the binding specificity of the surrogate antibody population to protein X could be enhanced by using protein Y as a non-specific moiety. In this way, a surrogate antibody population with enhanced interaction to protein X could be produced. See, for example, Giver et al. (1993) Nucleic Acid Research 23: 5509-5516 and Jellinek et al. (1993) Proc. Natl. Acad. Sci 90:11227-11231.

VI. Method of Enhancing the Binding Affinity of a Surrogate Antibody or a Population Thereof

Binding affinity is a term that describes the strength of the binding interaction between the surrogate antibody and a ligand. An enhancement in binding affinity results in the increased binding interaction between the target ligand and the surrogate antibody. The binding affinity of the surrogate antibody and target ligand interaction directly correlates to the sensitivity of detection that the surrogate antibody will be able to achieve. In order to assess the binding affinity under practical applications, the conditions of the binding reactions must be comparable to the conditions of the intended use. For the most accurate comparisons, measurements will be made that reflect the interaction between the surrogate antibody and target ligand in solutions and under conditions of their intended application.

Accordingly, the present invention provides method of enhancing the binding affinity of a surrogate antibody comprising:

a) contacting a ligand with a population of surrogate antibody molecules under stringent conditions that permit formation of a population of ligand-bound surrogate antibody complexes,

wherein said surrogate antibody molecule of the surrogate antibody population comprises a specificity strand and a stabilization strand,

said specificity strand comprising a nucleic acid sequence having a specificity region flanked by a first constant region and a second constant region; and,

said stabilization strand comprises a first stabilization domain that interacts with said first constant region and a second stabilization domain that interacts with said second constant region;

b) partitioning said ligand, said population of surrogate antibody molecules from said population of ligand-bound surrogate antibody complexes; and,

c) amplifying the specificity strand of said population of ligand-bound surrogate antibody complexes.

In a further embodiment, the method of enhancing binding affinity further comprises contacting said population of specificity strands of step (c) above with a stabilization strand under conditions that allow for said first stabilization domain to interact with said first constant region and said second stabilization domain to interact with said second constant region.

In further embodiments, the population of surrogate antibodies comprise a library of surrogate antibodies and/or a population of selected surrogate antibodies. In other embodiment, the stabilization strand and the specificity strand comprise distinct molecules.

In this embodiment, contacting the desired ligand with a population of surrogate antibody molecules under stringent conditions that permit formation of a population of ligand-bound surrogate antibody complexes, allows for the selection of surrogate antibodies that have increased binding affinity to the desired ligand. By “stringent conditions” is intended any condition that will stress the interaction of the desired ligand with the surrogate antibodies in the population. Such conditions will vary depending on the ligand of interest and the preferred conditions under which the surrogate antibody and ligand will interact. It is recognized that the stringent condition selected will continue to allow for the formation of the surrogate antibody structure. Examples of such stringent conditions include changes in osmolarity, pH, solvent (organic or inorganic), temperature surfactants, or any combination thereof. Additional components that can produce stringent conditions include components that compromise hydrophobic, hydrogen bonding, electrostatic, and Van der Waals interactions. For example, 10% methanol or ethanol compromise hydrophobic boning and are water soluble.

The stringency of conditions can also be manipulated by the surrogate antibody to ligand ratio. This increase can occur by an increase in surrogate antibody or by a decrease in target ligand. See, for example Irvine et al. (1991) J Mol Biol 222:739-761. Additional alterations to increase the stringency of binding conditions include, alterations in salt concentration, binding equilibrium time, dilution of binding buffer and amount and composition of wash. The stringency of conditions will be sufficient to decrease the % antibody bound by 1% to 10%, 10% to 20%, 20% to 30%, 30% to 40%, 40% to 50%, 60% to 70%, 70% to 80%, 80% to 90%, 90% to 99% of the total population.

In yet other embodiments, following the identification and isolation of a monoclonal surrogate antibody that has desirable ligand binding specificity, one of skill could further enhance the affinity of the molecule for the desired purpose by mutagenesizing the specificity region and screening for the tighter binding mutants. See, for example, Colas et al. (2000) Proc. Natl. Aca. Science 97:13720-13725.

Methods of Use

As discussed above, the surrogate antibodies and various populations of surrogate antibodies (i.e., libraries, selected populations, polyclonal populations, and monoclonal surrogate antibody populations) described herein interact with a desired ligand. As such, ligand-binding surrogate antibodies can be used to replace conventional antibodies in testing, pharmaceutical, and research applications. Modifications that can be introduced into their loop size, number of binding loops, conformation, stabilization strand and nucleotide chemistry provides a greater binding than is present with conventional antibodies. Accordingly, the surrogate antibodies of the invention can be used in a variety of methods including methods to modulate ligand activity. Also, provided are methods for the isolation of proteins or other molecule that interacts with the ligand.

As used herein, “ligand” is intended to be any molecule that forms a complex with another molecule, such as the target antigen of a precipitation assay, flocculation, agglutination or immunoassay. A ligand therefore includes an ion, a molecule, or a molecular group that binds to another chemical entity to form a larger complex. It is recognized that in the various methods described above, more than one target ligand can be used to simultaneously capture a plurality of surrogate antibodies from a starting library or population or to enhance binding specificity of the population of antibodies. The ligands can differ from one another in their surrogate antibody binding affinities and can act as an agonist, antagonist, partial agonist, inverse agonist or allosteric modulator.

A ligand therefore will encompass any desired molecule that interacts with a surrogate antibody. A target molecule or ligand can be a cell and/or its constituents. Any cell type of interest, at any developmental stage of interest, and having various phenotypes and pathological condition, such as cancerous phenotypes can be used. Cells of interest further include prokaryotic cells or eukaryotic cells such as epithelia cells, muscle cells, secretory cells, malignant cells and erythroid and lymphoid cells. Other ligands of interest include, a toxic environmental compound, a nucleic acid, a protein, a peptide, natural and synthetic polymers, a carbohydrate, a polysaccharide, a mucopolysaccharide, a glycoprotein, a hormone, a receptor, an effector, an enzyme, an antigen, an antibody, a bacteria and its constituents, including but not limited to, Francisella tularensis including, Francisella tularensis holarctica, Francisella tularensis mediasiatica, Francisella tularensis novicida, and Francisella tularensis tularensis., a virus, a protozoa, a prion, a substrate, a metabolite, a small molecule, a drug, a narcotic, a toxin, a transition state analog, a cofactor, an inhibitor, a dye, a nutrient, a growth factor, a unique cell surface determinant or intracellular marker, etc., without limitation. Ligands can further include immunological haptens, toxic environmental compounds such as, polychlorinated biphenyls, substances of abuse, therapeutic drugs and thyroxin. Additional ligands of interest include molecules whose levels are altered in tumors (i.e., growth factor receptors, cell cycle regulators, angiogenic factors, and signaling factors). Accordingly, the surrogate antibody molecules of the invention can be produced for the detection of any ligand of interest.

For example, surrogate antibody molecules can be used to bind proteins, including both nucleic acid-binding proteins and proteins not known to bind nucleic acids as part of their biological function. Nucleic acid binding proteins include among many others polymerases and reverse transcriptases. The surrogate antibody molecules can also be used to bind nucleotides, nucleosides, nucleotide co-factors and structurally related molecules.

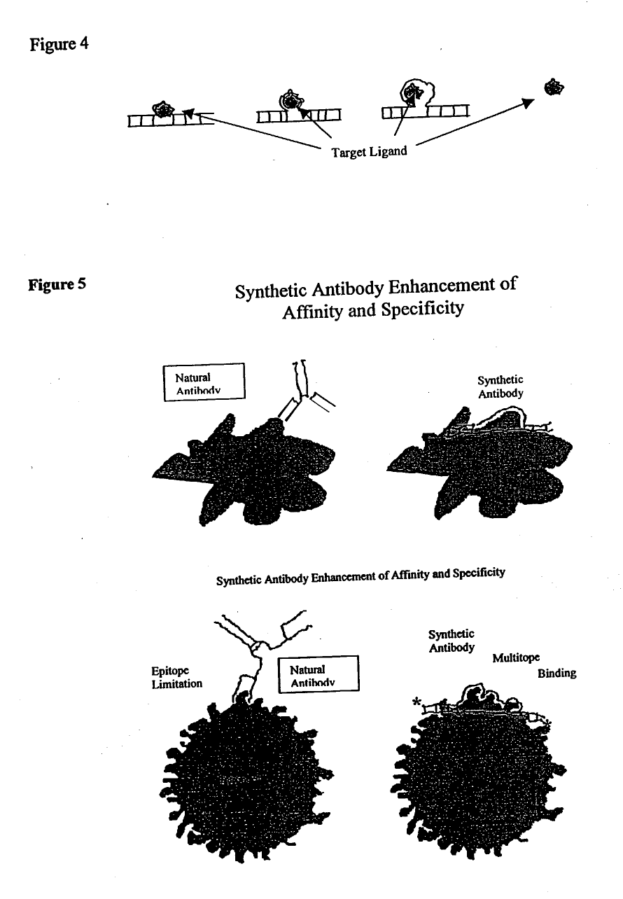

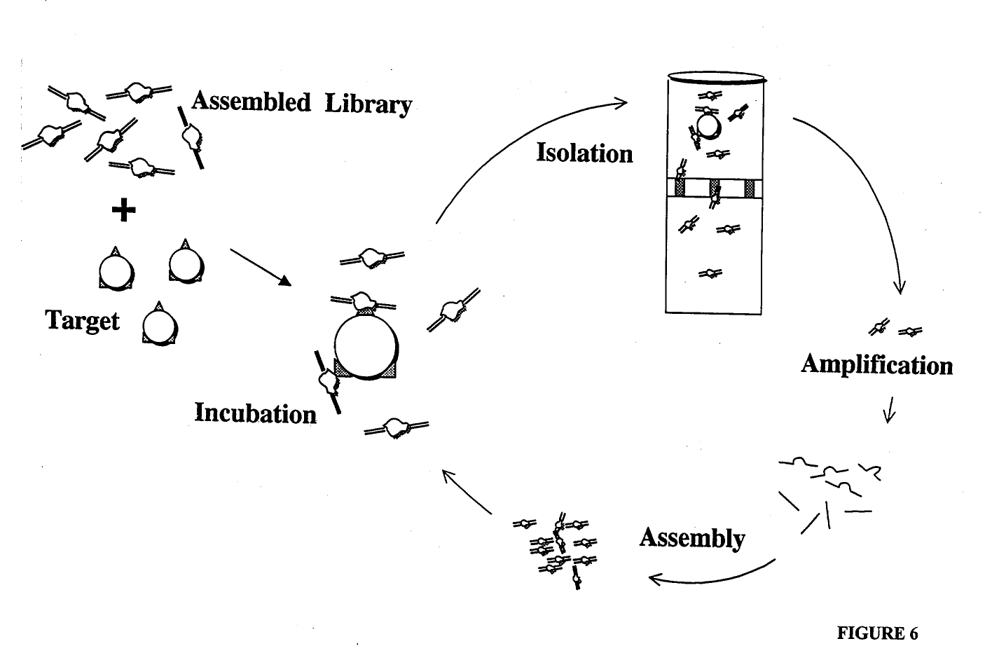

An “epitope” or “determinant” is the site on a ligand to which a natural antibody molecule binds. The size of an epitope is limited by the dimensions of the antibody-binding cavity, and can accommodate a molecule up to approximately 4 amino acids or 6 glucose molecules in size. The binding site dimension of a natural antibody allows the recognition of unique features (epitope) of a relatively small size. They are unable to identify features that may exist outside of this binding site limitation (see FIG. 4).

Moreover, the surrogate antibodies can be used to detect a plurality of compounds or organisms simultaneously, or used in a profiling array for multi-parametric detection and quantification. They can be used to prepare an environmental testing array to detect related compounds (e.g. PCB congeners), or dissimilar compounds that have adverse environmental or health effects (e.g. PCBs, Dioxins, Polyaromatic Hydrocarbons). Surrogate antibodies can be developed to bind normal, abnormal, or unique constituents found on or within prokaryotic cells (e.g. bacteria), viruses, eukaryotic cells (e.g. epithelial cells, muscle cells, nerve cells, sensory cells, secretory cells, malignant cells, erythroid and lymphoid cells, stem cells, protozoa, fungi). They can be used to identify and detect tumor-associated antigens, cancer cells or unique structures or compounds associated with specific disease cells.

Surrogate antibody molecules can be produced to ligands that would not stimulate an immune response because of limited size, complexity, foreignness to host, or genetic limitation in the host. They can be produced to compounds that are toxic to antibody producing organisms or cell cultures.

Any molecule or collection of molecules could be used to develop a surrogate antibody that interacts with the molecule. In fact, the criteria for producing surrogate antibody molecules is that the target ligand-surrogate antibody complex assumes a physico-chemical characteristic that is different than that of the uncomplexed surrogate antibody molecule. An example being the increase in size of a surrogate antibody ligand complex compared to the size of the uncomplexed surrogate antibody molecule, and the use of size exclusion filtration to separate bound from free. In this example, surrogate antibody molecules are produced to ligands that when bound to surrogate antibodies are retained by the porosity of a filter membrane, while uncomplexed surrogate antibody molecules proceed into the filtrate.

I. Methods of Detecting a Ligand

A method of detecting a ligand is provided. In one embodiment, the method of detecting a ligand comprises

a) contacting the ligand with a surrogate antibody molecule under conditions that permit formation of a ligand-bound surrogate antibody complex, wherein said surrogate antibody molecule comprises a specificity strand and a stabilization strand,

the specificity strand comprising a nucleic acid sequence having a specificity region flanked by a first constant region and a second constant region; and,

the stabilization strand comprising a first stabilization domain that interacts with the first constant region and a second stabilization domain that interacts with the second constant region; and,

b) detecting said ligand.

Methods of contacting and conditions that permit formation of a ligand-bound surrogate antibody have been discussed elsewhere herein. One of skill will recognize that the specific reaction conditions will vary depending on the reaction chemistry and experimental design.

By “detecting” is intended the identification of the ligand-bound surrogate antibody complex. The method of detection is not restricted and may be either qualitative or quantitative. As discussed in detail above, a variety of functional moieties can be attached directly to the surrogate antibody that will aid in the detection of the ligand-bound surrogate antibody complex, including for example, enzymes such as Alkaline Phosphatase, Horseradish Peroxidase, or radiolabels, fluorophores, chemiluminescence, etc. See, for example, Mayer et al. (2001) Proc. Natl. Acad. Sci. 98:4961-4965 that describes the detection of a RNA/protein interaction.

Alternatively, a two-site binding assay can be used to detect the ligand. In this embodiment, the ligand-surrogate antibody complex is bound to a second “detector” molecule. Such types of sandwich assays are known in the art. See, for example, Drolet et al. (1996) Nat. Biotechnology 14: 1021-5, which detected fluoroscein attached to the 5′ end of a nucleic acid molecule using a Fab fragment conjugated to alkaline phosphatase. See, also, Jenion et al. (1995) Antisense Nucleic Acid Drug Dev 8:265-79 and Bock et al. (1992) Nature 355:564-6.

Before forming the ligand-bound surrogate antibody complex, the surrogate antibody (for example, a unselected library, or various other types of populations) can be immobilized to a plurality of locations on solid matrices, such as plastic or glass plates, tubes, membranes, or sensor chips, for the purpose of facilitating the rapid capture and amplification of surrogate antibody molecules or for the purpose of identifying bound ligands (e.g. for high throughput drug discovery). See, Green et al. (2001) Biotechniques 30:1084-6. A solution containing the ligand is added thereto.

Alternatively, the surrogate antibody and ligand can be mixed together in a solution, the ligand-bound surrogate antibody complex is formed. Before being detected, the ligand-bound surrogate antibody complex can be separated from other impurities. For example, centrifuge and affinity chromatography can be employed. Separation is not necessarily required. See, also, Jhaveri et al. (2000) J. Am. Chem. Soci. 122:2469 that demonstrates that apatmer-dye conjugates can directly signal the presence of ligand in solution without the need for prior immobilization and washing. For example, fluorophores modified nucleotides in the binding cavity can quench upon ligand binding. This technique could also be used to identify critical residues of specificity regions involved in ligand binding.

Surrogate antibody molecules can be used in binding assays that are used to detect, identify, and/or quantify ligands using a heterogeneous binding assay that involves one or more washing steps used to separate surrogate antibodies that are bound to a target ligand, or conjugated form of the target ligand, from surrogate antibodies that are not bound to the ligand, or conjugated form of the target ligand. See, for example, Wang et al. (1996) Biochemistry 12:338-46 and Tyagi et al. (1998) Nat Biotechnol 16:49-53.

Surrogate antibody molecules can be used in binding assays that are used to detect, identify, and/or quantify ligands using a homogeneous binding assay that involves the modulation of signal produced as a result of surrogate antibody molecules binding to the target ligand, or conjugated form of the target ligand. See, for example, Wilson et al. (1998) Clin Chem 44:86-91; Patel et al. (1997) J Mol Bio 272:645-64; Hsiung et al. (1996) Nat. Struct Biol 3:1046-50; Tyagi et al. (1996) Nat Biotech 14:303-8, and Tyagi et al. (1998) Nat. Biotech 16:49-53 and Fang et al. (2001) Anal Chem 73:5752-7.

Accordingly, the surrogate antibody molecules can facilitate the development of high throughput assays, the identification of cancer and other markers (i.e., those markers associated with various pathological conditions), and the detection of immunological antigens and haptens. The surrogate antibodies can be used in the same or similar manner as antibodies in conventional antibody-based immunoassays.

Surrogate antibodies can be used to identify new diagnostic markers of disease (e.g. cancer), wherein surrogate antibody molecules (i.e., populations of monoclonal or selected populations of surrogate antibodies or polyclonal antibodies) are produced to unique elements on, or within, a cancer cell. Such surrogate antibody molecules can be labeled with a reporter molecule (e.g. FITC) and used to identify the prevalence of the detected element on the cancer cells of different individuals. The incidence of detection of such a marker can be recorded in a database. Methods of administering are discussed elsewhere herein.

In specific embodiments, the ligand is detected within a cell, tissue, organ, or organ system.

It is recognized that the ligand may be detected either in vitro or in vivo. For example, tissues, cells, or organ systems containing the ligand of interest within or on their surface can be contacted in vitro with the appropriate surrogate antibody. The ligand-bound surrogate antibody complexes can then be detected. Thus, in one embodiment, the invention relates to a pharmaceutical composition comprising a surrogate antibody or a population of surrogate antibodies as described herein.

In another embodiment, the invention relates to a pharmaceutical composition comprising a surrogate antibody or a population of surrogate antibodies as described herein. In one method, such a compositions could be used for in vivo detection of a pathological condition that is characterized by, for example, either an increased or a decreased level of the ligand. In this method, a subject is administered an effective amount of a surrogate antibody having the binding specificity for a ligand whose concentration is elevated or decreased in a particular pathological condition. Formation of the ligand-bound surrogate antibody is detected.

The term “pathological condition” refers to an abnormality or disease, as these terms are commonly used in the art. A non-limiting list of such conditions comprises cancer, arthritis, septicemia, myocardial arrhythmias and infarctions, viral and bacterial infections, autoimmune, and prion diseases.

II. Method of Modulating the Activity

Further provided are methods of modulating the activity of a ligand. By “modulating” or “modulation” is intended an increase or a decrease in a particular character, quality, activity, substance, or response.

In one embodiment, the method of modulating ligand activity comprises contacting the ligand with a surrogate antibody molecule under conditions that permit formation of a ligand-bound surrogate antibody complex, wherein said surrogate antibody molecule of the surrogate antibody comprises a specificity strand and a stabilization strand, a) the specificity strand comprising a nucleic acid sequence having a specificity region flanked by a first constant region and a second constant region; and, b) the stabilization strand comprises a first stabilization domain that interacts with said first constant region and a second stabilization domain that interacts with said second constant region. The interaction of the ligand with the surrogate antibody modulates the activity of the ligand or modulates the activity of a molecule conjugated to the ligand. In this embodiment, an effective concentration of surrogate antibody is used so as to allow the desired modulation of ligand activity to occur. In another embodiment, the specificity stand and the stabilization strand comprise distinct molecules.

It is recognized that the modulation may occur either in vivo or in vitro. In addition, the ligand may be contained within a cell, tissue, organ, or organ system. Methods for assaying the ability of a surrogate antibody molecule to modulate ligand activity are known in the art (i.e., fluorophore polarization assays, interference and complementation assays, interference of enzyme or substrate activity, or alteration of light refractive properties). In addition, the interaction can be monitored in vitro and the activity of the ligand assayed. Alternatively, the modulation of ligand activity can be assayed in vivo.

The activity of a variety of ligands can be modulated by the this method, including, for example, receptors, effectors, enzymes, hormones, transport proteins, inorganic molecules, organic molecules, virus, bacteria, protits, or prions. Methods to assay for the modulation of ligand activity will vary depending on the ligand. One will further recognize the assay could directly measure ligand activity or alternatively, the phenotype of the cell, tissue or organ could be altered. Consequently, the ligand is on or within a cell, tissue, organ, or organ system.

Thus, in one embodiment, surrogate antibody reagents can be used to modulate the function of a target molecule. In one embodiment, surrogate antibody molecules bound to a particular receptor function as agonists, antagonists, inverse agonists, partial agonists, or allosteric modulators. In addition, the surrogate antibody may act as a mimotype (see U.S. Pat. No. 5,874,563). Where the target molecule is an enzyme the surrogate antibody molecules can be used to inhibit or augment enzyme activity.

In one embodiment, an immune response is modulated, either via a direct interaction with the ligand of interest or via an indirect modulation of the immune response that occurs following interaction with the ligand of interest.

In another embodiment, the surrogate antibodies are used to “pan” disease cells for the purpose of binding epitopes and accelerating apoptosis of for the identification of unique cipitopes for drug delivery. In addition, the apoptogenic epitopes will also be used for in vitro rapid drug discovery. Thus, the surrogate antibodies find use in modulating the activity of apoptotic epitopes and thereby modulating (i.e., enhancing or delaying) cell death.

III. Methods of Delivering an Agent

The surrogate antibody molecules of the invention may be mono-, bi- or multi-functional molecules. In one embodiment the surrogate antibody functions as a transport and delivery vehicle. Accordingly, further provided are methods for delivering an agent of interest. By “agent” is intended any auxiliary molecule and thus encompasses the various functional moieties described above, including for example a “reporter” molecule that can amplify the detection ability of the surrogate antibody when used in binding assays; “therapeutic” molecules that are delivered to a specific site; or, “binding molecules” that facilitate the attachment of a broad array of ligands. “Reporter” molecules can be added, for example, using chemically modified primers, by direct chemical methods, or by complex formation to a “binding molecule” (affinity tags) incorporated in the stabilization or specificity strands.

Thus, the present invention provides a method of delivering an agent comprising contacting a ligand with a surrogate antibody molecules under conditions that permit formation of a population of ligand-bound surrogate antibody complexes, wherein said surrogate antibody molecule of the surrogate antibody population comprises a specificity strand and a stabilization strand. The surrogate antibody comprises a specificity strand comprising a nucleic acid sequence having a specificity region flanked by a first constant region and a second constant region; and, a stabilization strand comprises a first stabilization domain that interacts with said first constant region and a second stabilization domain that interacts with said second constant region. The surrogate antibody further has attached thereto or comprises the agent of interest.

Therapeutic agents include, for example, those pharmaceutical compounds that are developed for use in the treatment of cancer, arthritis, septicemia, myocardial arrhythmia's and infarctions, viral and bacterial infections, autoimmune disease and prion diseases. In this manner, surrogate antibodies can be used as therapeutic targeting agents when complexed to one or more therapeutic agent(s) that can be the same agent or different agent(s).

When the agent of interest is to be delivered to treat a particular disorder, the therapeutic agents can be selected for the particular disorder. For example, where the surrogate antibodies are targeted to a unique tumor antigen found on a tumor cell at a specific tumor site, the surrogate antibodies can be conjugated to an anti-tumor agent for specific delivery to that site and to minimize or eliminate collateral pathology to normal tissue. The agent can be delivered to a specific target ligand recognized by the surrogate molecule and found specifically at the tumor site.

The therapeutic agents can be virtually any type of anti-tumor or anti-angiogenic compound (i.e., an agent that disrupts the vasculature supplying a tumor) that can be attached to the surrogate antibody, and can include, for purpose of example, synthetic or natural compounds such as cytotoxin, interleukins, chemotactic factors, radioneucleotides, methotrexate, cis-platin, anastrozole/Arimidexg and tamoxifen. Additional agents include biological toxins such as ricin or diptheria toxin, fungal-derived calicheamicins, maytansinoids, Pseudomanas exotoxins, and ribosomes inactivating proteins. See, for example, Buschsbaum et al. (1999) Clin. Cancer Res 5: Grassband et al. (1992) Blood 79:576-83; Batra et al (1991) Mol Cell Biol. 11:2200-5; Penichet et al. (2001) J Immunol Meth 248:91-101; Hinman et al. (1993) Cancer Res 53:3336-3342; Tur et al. (2001) Intt J Mol Med 8:579-584; and Tazzari et al. (2001) J Immunol 167:4222-4229.

Alternatively, the therapeutic agent could comprise a prodrug. After its localization to the specific target, a non-toxic molecule is injected that coverts the prodrug to a drug. See, for example, Senter et al. (1996) Advanced Drug Delivery 22:341-9.

In one embodiment, the surrogate antibody molecules having a nucleic acid composition, as opposed to the protein composition of native antibody molecules or antibody fragments used currently to deliver therapeutic agents, are significantly less immunogenic and are less likely to be eliminated by the patient by evoking an immune response. It is further recognized, surrogate antibodies having a stabilization strand composed of peptides for the stabilization domains may also be less immunogenic by humanizing the sequence and/or decreasing the size of the peptide required to form the stabilization domain.

Accordingly, one embodiment of the invention provides for directing an agent to a desired location via the interaction of the surrogate antibody molecule and its target ligand. In one embodiment, the method of delivering an agent comprises contacting a ligand with a surrogate antibody molecule under conditions that permit formation of a ligand-bound surrogate antibody complex, and thereby deliver the associated therapeutic agent to the desired target site (i.e., site of pathology). Such surrogate antibody molecules can be used unmodified, or modified with nuclease-resisting bases, or by any of the diverse structures discussed elsewhere herein.

In one embodiment, the agent attached to the surrogate antibody comprises a molecule having anti-microbial activity. By “anti-microbial activity” is intended any ability to inhibit or decrease the growth of a microbe and/or the ability to decrease the number of microbes in a microbial population. By “microbe” in intended a bacterial, virus, fungi, or parasite and consequently, the agent having anti-microbial activity possess anti-bacterial activity, anti-fungal activity, and/or anti-viral activity.

By “anti-bacterial activity” is intended any ability to inhibit or decrease the growth of a bacteria and/or the ability to decrease the number of viable bacterial cells in a bacterial population. The agent can be a Gram-positive anti-bacterial agent, a Gram-negative anti-bacterial agent, or a male specific anti-bacterial agent. By “anti-viral activity” is intended any ability to inhibit or decrease the growth of a virus or a virus infected cell and/or the ability to decrease the population of viable viral particles or virally infected cells in a population. The term “anti-fungal activity” is intended the ability to inhibit or decrease the growth of fungi. Anti-microbial agents are known in the art and include various chemokines, cytokines, anti-microbial polypeptides (i.e., anti-bacterial, anti-viral, and anti-fungal polypeptides), antibiotics, LPS, complement activators, CpG sequence, and various other agents having anti-microbial activity. Exemplary anti-microbial agents are discussed in further detail below.

Accordingly, in one embodiment, the present invention provides a surrogate antibody covalently attached to an anti-microbial agent. Using the various methods described herein, the antibody can be designed to bind to a specific target ligand (i.e., an epitope of the target microbe). The surrogate antibody/anti-microbial complex can then be used as a means to delivered the anti-microbial agent to the microbe. The compositions find use in in vitro applications as a method to decrease anti-microbial titer in various samples, including tissue culture. Thus, the surrogate antibody molecule can be used as an additive for in vitro cell cultures to prevent the overgrowth of microbes in tissue culture. In addition, the compositions find use as a therapeutic agent that, upon administration to a subject in need thereof, will inhibit or decrease the growth of a microbe contained within said subject and/or decrease the microbial population in the subject.

Chemokines comprise one class of anti-microbial agents that could be used in the methods and compositions of the invention. Multiple classes exist including CC chemokines (i.e., MCP-1 (SwissPro Accession No. P13500 and U.S. Pat. No. 6,132,987) and CXC chemokines (i.e., IL8 (SwissPro Accession No. P10145), IP-10 (SwissPro Accession No. P02778). In addition, granulysin in another chemokine of interest. This polypeptide is produced by cytolytic T-lymphocytes and natural killers cells and is active against a broad range of microbes including Gram-positive and Gram-negative bacteria, parasites, and Mycobacterium tuberculosis. Active variants and fragments of granulysin are known. See, for example, Kumar et al. (2001) Expert Opin Invest Drugs 10:321-9 and Anderson et al. (2003) J. Mol. Biol. 325:355-65, U.S. Pat. No. 4,994,369, U.S. Pat. No. 6,485,928, and GenBank Acc. Nos. X05044, X05044, and X541101, all of which are herein incorporated by reference.

Cytokines comprise another class of anti-microbial polypeptides that could be used in the methods and compositions of the invention. Multiple cytokines having anti-microbial activity are known in the art and include TNF-α, lymphotoxin (LT and TNF-β), IFN-γ, interleukin 12, etc.

Antibiotics comprise yet another class of anti-microbial polypeptides that could be used in the methods and compositions of the invention. Antibiotics of interest, include, but are not limited to penicillin, e.g. penicillin G, penicillin V, methicillin, oxacillin, carbenicillin, nafcillin, ampicillin, etc.; cephalosporins, e.g. cefaclor, cefazolin, cefuroxime, moxalactam, etc.; carbapenems; monobactams; aminoglycosides; tetracyclines; macrolides; lincomycins; polymyxins; sulfonamides; quinolones; cloramphenical; metronidazole; spectinomycin; trimethoprim; vancomycin; gentamicin; and ciprofloxacin HCL, ect.

Additional anit-microbial agents include Gram-positive anti-bacterial agents include, for example, members of the gallidermin protein family (InterPro Accession No. IPR006078). Such polypeptides include lantibiotics that are heavily modified bacteriocin-like peptides from Gram-positive bacteria. Type A lantibiotics include nisin (Interpro Accession No. IPR000446, P13068, P10946, and Kuipers et al (1998) J. Biol. Chem. 267:24340-24346), subtilin, epidermin, gallidermin (IPR Accession No, 006078, and GenBank Accession No. 068586, P08136, and P21838) and Pep5. These peptides are strongly cationic and bactericidal. See, for example, GenBank Accession No.068586, P08136, P21838 and Buchman et al. (1988) J. Biol. Chem. 263: 16260-16266, and Freund et al. (1991) Biopolymers 31:803-811. Each of these references is herein incorporated by reference.

Many other families of anti-microbial peptides are known. For example, the attacin polypeptide family has a conserved signature sequence as shown in PFAM Accession No. PF03769 and PF03768 and include polypeptides such as, attacin and sarcotoxin. See, for example, GenBank Acc. No. P01512 ATTB_HYACE, P01513 ATTE_HYACE, P10836 DIPA_PROTE, P14667 SR2_SARPE and Hoffmann et al. (1995) Curr. Opin. Immunol 7:4-10. Diptericin is another class of anti-microbial proteins. These polypeptides have some similarity to the attacin family. Diptericin-type polypeptides have been isolated from P. terranovae and S. peregina (Ishikawa et al. (1992) Biochem J. 287:573-578) and from D. melanogaster. Conserved regions along with active variants are known. See, for example, Otvos et al. (2000) J. Peptide Sci 6:497-511.

Cecropins are yet another class of potent anti-microbial proteins. See, for example, Boman et al. (1987) Annu. Rev. Microbiol. 41: 103-126, Boman et al. (1991) Cell 65: 205-207, Boman et al. (1991) Eur. J. Biochem. 201: 23-31, Boman et al. (1991) Eur. J. Biochem. 201:23-31, and Steiner et al. (1981) Nature 292:246-248. Cecropins are small proteins of about 35 amino acid residues active against both Gram-positive and Gram-negative bacteria. Cecropins isolated from insects other than Cecropia have been given various names including bactericidin, lepidopteran, sarcotoxin, etc. All of these peptides are structurally related and comprises the cecropin family signature (See PFAM Accession No. PF00272). Members of the family include GenBank Accession Nos. Q94557 CECIDROV1 from Drosophila, P50720 CE3D_HYPCU from Hypantria cunea, Q27239 CECA_BOMMO from Bombyz mori, P14667 CECI_PIG from pig, and P08377 SRIC_SARPE from Sarcophaga peregrina. Each of these references is herein incorporated by reference

Defensins are a family of cysteine-rich anti-bacterial peptides, primarily active against Gram-positive bacteria. Many of these peptides range in length from 38 to 51 amino acids and contain six conserved cysteines all involved in intrachain disulfide bonds. See, for example, Lambert et al. (1989) Proc. Natl. Acad. Sci. U.S.A. 86:262-266, Keppi et al. (1989) Proc. Natl. Acad. Sci. U.S.A. 86: 262-266. Fujiwara et al. (1990) J. Biol. Chem. 265: 11333-11337, Yamada et al. (1993) Biochem. J. 291: 275-279, Bulet et al. (1991) J. Biol. Chem. 266: 24520-24525, Bulet et al. (1992) Eur. J. Biochem. 209: 977-984(1992), Hanzawa et al. (1990) FEBS Lett. 269: 413-420, Cociancich et al. (1993) Biochem. Biophys. Res. Commun. 194:17-22, Hughes et al. (1999) Cell. Mol. Life Sci. 56:94-103, Cociancich et al. (1994) Biochem. J. 300: 567-575, Hoffmann et al. (1992) Immunol Today 13: 411-415, Dimarcq et al. (1994) Eur. J. Biochem 221:201-209, and Lowenberger et al. (1995) Insect Biochem. Mol Biol. 25: 867-873. Exemplary Arthropod defensins include, but are not limited to; P17722 DEFI APIME (Royalisin) from the royal jelly of honey bee, P31529 SAPB_SARPE sapecin B from flesh fly (Sarcophaga peregrina), P18313 SAPE_SARPE Sapecin from flesh fly (Sarcophaga peregrina), P41965 DEF4_LEIQH 4 Kd defensin from the scorpion Leiurus quinquestriatus hebraeus, P80154 DEFI_AESCY Defensin from the larva of the dragonfly Aeschna cyanea P10891 DEFI_PROTE Phormicin A and B from black blowfly (Protophormia terraenovae), P37364 DEFI_PYRAP: Defensin from Pyrrhocoris apterus, P31530 SAPC_SARPE sapecin C from flesh fly (Sarcophaga peregrina), and P80033 DEFA_ZOPAT anti-bacterial peptides B and C from the beetle Zophobas atratus. Each of these references is herein incorporated by reference. Several mammalian defines are also known. See, for example, Porter et al. (1997) Infection and Immunity 65:2396-2401.

Drosocin are another family of anti-microbial polypeptides. Members of this family have been identified and include pyrrhocoricin from Pyrrhocoris apterus (Coclancich et al. (1994) Biochem J. 300:567-575), apidaceins from honey bees (Casteels et al. (1989) EMBO J. 8:2387-2391) (discussed below), formaecin from Myrmecia gulosa (Mackintosh et al. (1989) J. Biol. Chem. 273:769-774). Other members include abaecin (Hara et al. (1995) Biochem J. 310:651-656) and lebocin (Furukawa et al. (1997) Biochem. Biophys. Res. Commun. 238: 796-774). Conserved domains and functional variants of this family are known. See, for example, Otvos et al. (2000) J. Peptide Sci 6:497-511, Otvos et al. (2002) Cell Mol. Life Sci. 59:1138-50, and Gennaro et al. (2002) Curr Pharm Des 8:763-78. Apidaecin are another family of anti-bacterial proteins found in bees and have the signature sequence of PFAM Accession No. 008807. These polypeptides possess anti-microbial activity against some human pathogens (Casteels et al. (1989) EMBO J. 8:2387-2391). Members of this family include GenBank Accession NO. P35581 AP22_APIME.

Cathelicidin are a family of anti-microbial polypeptides and have the signature sequence of PFAM Accession No. 000666. Many members of the family are secreted by neutrophiles upon activation. See, for example Zanetti et al. (1995) FEBS Letts 374:1-5. Members of this family include GenBank Accession No. P26202 (rabbit p15), P80054 (pig anti-bacterial peptide PR-39), P54228 (Bovine myeloid antibacterial peptide BMAP-27, P33046 Bovine indolicidin, a tryptophan-rich potent antibiotic, P49913 (human FALL-39 (or LL-37) an anti-bacterial LPS-binding peptide), P19660 (bovine bactenecin 5 (Bac5) proline and arginine rich antibiotics), P51437 (mouse CRAMP (CPL)), P32194 pig protegrin −1 to 5), P49930 (pig myeloid antibacterial peptides PMAP-23), P25230 (rabbit CAP18, a protein that binds to LPS), P15175 (pig cathelin), P49928 (sheep myeloid antibacterial peptide SMAP-29, and P54230 (sheep cyclic dodecapeptide, an antibiotic).

Additional anti-microbial peptides of interest include magainin. Active variants and fragments of this polypeptide are known. See, Ge et al. (1999) Antimicrobial Agents and Chemotherapy 43:782-788. For example, pexiganan comprises a variant of magainin having multiple substitutions and deletions that continues to possess anti-microbial activity and is currently used as a therapeutic anti-microbial agent for the topical treatment of infected diabetic foot ulcers (Lipsky et al. (1997) In Program and abstracts of the 37^{th} Interscience Conference on Antimicrobial Agents and Chemotherapy. American Society for Microbiology, Washington, D.C. Another anti-microbial polypeptide includes Vimetin. See, for example, Nirit et al. (2003) Nature Cell Biology 5:59-63. Each of these references is herein incorporated by reference.

It is recognized that when the anti-microbial agent comprises an anti-microbial peptide, the peptide can be from any animal species including, but not limited to, insects, rodent, avian, canine, bovine, porcine, equine, and, human. In some embodiments, the anti-microbial peptide administered is from the same species as the subject undergoing treatment.

Biologically active variants of anti-microbial polypeptides and biologically active derivatives of anti-microbial agents are also encompassed by the methods of the present invention. Such variants and derivatives should retain the biological activity of the anti-microbial agent (i.e., anti-microbial activity, anti-bacterial activity, anti-viral activity and/or anti-fungal activity). Active variants of such sequences are known in the art as are method to assay for the activity. Preferably, the variant has at least the same activity as the native molecule.

Suitable biologically active variants of an anti-microbial polypeptide can be fragments, analogues, and derivatives of the anti-microbial polypeptides. By “fragment” is intended a protein consisting of only a part of the intact anti-microbial polypeptide sequence. The fragment can be a C-terminal deletion or N-terminal deletion of the regulatory polypeptide. By “variant” of an anti-microbial polypeptide is intended an analogue of either the full length polypeptide having anti-microbial, anti-viral, anti-bacterial, and/or anti-fungal activity, or a fragment thereof, that includes a native sequence and structure having one or more amino acid substitutions, insertions, or deletions. Peptides having one or more peptoids (peptide mimics) are also encompassed by the term analogue (see i.e., International Publication No. WO 91/04282).

By “derivative” of an anti-microbial agent is intended any suitable modification of the native anti-microbial polypeptide or fragments thereof, their respective variants or any suitable modification of the native anti-microbial agent, such as glycosylation, phosphorylation, or other addition of foreign moieties, so long as the activity is retained.

Preferably, naturally or non-naturally occurring variants of an anti-microbial polypeptide have amino acid sequences that are at least 70%, preferably 80%, more preferably, 85%, 90%, 91%, 92%, 93%, 94% or 95% identical to the amino acid sequence to the reference molecule, for example, an anti-microbial peptide such as granulysin, or to a shorter portion of the reference anti-microbial polypeptide. More preferably, the molecules are 96%, 97%, 98% or 99% identical. Percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant may, for example, differ by as few as 1 to 10 amino acid residues, such as 6-10, as few as 5, as few as 4, 3, 2, or even 1 amino aid residue.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waternan homology search algorithm).

The art provides substantial guidance regarding the preparation and use of such variants. A fragment of an anti-microbial polypeptide will generally include at least about 10 contiguous amino acid residues of the full-length molecule, preferably about 15-25 contiguous amino acid residues of the full-length molecule, and most preferably about 20-50 or more contiguous amino acid residues of full-length anti-microbial polypeptide.

The anti-microbial agent attached to the surrogate antibody of the invention can be active against any microbe of interest. Microorganisms of interest include, but are not limited to aerobes including both Gram-positive aerobes and Gram-negative aerobes. Gram-positive aerobes include Staphylococcus sp., e.g. Staphylococcus aureus, Staphylococcus epidermidis, Staphylococcus haemolyticus, other coagulase-negative staphylococci, Streptococcus agalactiae, Streptococcus pyogenes, Streptococcus sanguis, other streptococci, Enterococcus faecalis, Enterococcus faecium, Clostridia sp., e.g. C. tetani, C. botulinum, Micrococcus spp., and Corynebacterium spp, e.g. C. diptheriae. Gram-negative aerobes include Acinetobacter baumanii, Alcaligenes faecalis, Citrobacter diversus, Citrobacter freundii, Enterobacter aerogenes, Enterobacter cloacae, Escherichia sp., e.g. E. coli; Klebsiella oxytoca, Klebsiella peeumoniae, Pseudomanas aeruginosa, other Pseudomanas spp., and Stenotrophomonas maltrophila.

Additional microbes of interest include anaerobes. Gram-positive anaerobes include, for example, Clostridium innocuum, Clostridium perfringes, Clostridium ramosm, Clostridiium sporogenes, Peptostreptococcus anaerobius, Peptostreptococcus magnus, Peptostreptococcus prevotii, Propionibacterium acnes. Gram-negative anaerobes include, for example, Baceroides distason is, Bacteroides fragilis, Bacteroides ovatus, Bacteroides thetaiotaomicron, Fusobacterium nucleatum, Prevotella bivia, and Prevotella melaniogenica.

Additional bacteria of interest include, Klebsiella sp., Morganella sp.; Proteus sp.; Providencia sp.; Salmonella sp., e.g. S. typhi, S. typhimurium; Serratia sp.; Shigella sp.; Pseudomonas sp., e.g. P. aeruginosa; Yersinia sp., e.g. Y. pestis, Y. pseudotuberculosis, Y. enterocolitica; Francisells sp.; Pasturella sp.; Vibrio sp., e.g. V. cholerae, V. parahemolyticus; Campylobacter sp., e.g. C. jejuni; Haemophilus sp., e.g. H. influenzae, H. ducreyi; Bordetella sp., e.g. B. pertussis, B. bronchiseptica, B. parapertussis; Brucella sp., Neisseria sp., e.g. N. gonorrhoeae, N. meningitidis, etc. Other bacteria include Legionella sp., e.g. L. pneumophila; Listeria sp., e.g. L. monocytogenes; Mycoplasma sp., e.g. M. hominis, M. pneumoniae; Mycobacterium sp., e.g. M. tuberculosis, M. leprae; Treponema sp., e.g. T. pallidum; Borrelia sp., e.g. B. burgdorferi; Leptospirae sp.; Rickettsia sp., e.g. R. rickettsii, R. typhi; Chlamydia sp., e.g. C. trachomatis, C. pneumoniae, C. psittaci; Helicobacter sp., e.g. H. pylori, etc.

Non bacterial microbes of interest include fungal and protozoan pathogens, e.g. Plasmodia sp., e.g. P. falciparum, Trypanosoma sp., e.g. T. brucei; shistosomes; Entaemoeba sp., Cryptococcus sp., Candida sp, e.g. C. albicans; etc.

Viruses of interest include, but are not limited to respiratory viral pathogens including, for example, adenovirus, echovirus, rhinovirus, cosackievirus, coronavirus, influenza A and B viruses, parainfluenza virus 1-4, respiratory syncytial virus. Digestive viral pathogens include, for example, the mumps virus, rotavirus, Norwalk Agent, hepatitis A virus, hepatitis B virus, hepatitis D virus and hepatitis C virus, and hepatitis E virus. Systemic viral pathogens include, for example, measles virus, rubella virus, parvovirus, varicella-zoster virus, herpes simplex virus 1-associated, and herpes simplex virus 2. Systemic viral pathogens include, for example, cytomegalovirus, Epstein-Barr virus, HTLV-1, HTLV-II; and HIV 1 and HIV 2. Arboviral pathogens include, for example, dengue virus 1-4, yellow fever virus, Colorado tick fever virus, and regional hemorrhagic fever viruses. Additional viral pathogens include, for example, papillomavirus and molluscum virus, poliovirus, rabiesvirus, JC virus, and arboviral encephalitis viruses. Viral pathogens associated with cancer include, for example, human papillomaviruses, Epstein-Barr virus, hepatitis B virus, human T-cell leukemia virus type 1 (HTLV-1), and the Kaposi sarcoma herpesvirus (KSHV).

Additional microbes of interest include tick-transmitted microbes. These include, for example, orthomyxovirus, lyme disease spirochetes (i.e., Borrelia burgdorferi, B. lusitaniae), tick-borne encephalitis (TBE) virus. Ticks further transmit the protozoan Babesia microti; B. divergens, B. bovis and B. bigemina, all known pathogens of cattle, (Despommier et al. (1995). Parasitic Diseases Springer-Verlag, New York. Additional microbes transmitted include rickettsial Ehrlichia species. In addition, a babesiosis-like illness in the northwestern United States has been attributed to an unidentified Babesia-like organism, thus far termed WA1. Quick et al. (1993) Annals of Int. Med. 119: 284-290 (1993).

Other microbes of interest include Francisella tularensis including, Francisella tularensis holarctica, Francisella tularensis mediasiatica, Francisella tularensis novicida, and Francisella tularensis tularensis.

The methods of the invention comprise contacting a surrogate antibody having an anti-microbial agent attached thereto to a microbe. The term “contacting” refers to exposing a microbe to the surrogate antibody so that the associated anti-microbial agent can effectively inhibit or kill the microbe. Contacting may be in vitro, for example, by adding the surrogate antibody to a bacterial culture to test for susceptibility of the microbe to the surrogate antibody complex or by adding the surrogate antibody to a cell culture to inhibit or kill contaminating microbes. Alternatively, the contacting may be in vivo, for example, administering the peptide to a subject having a microbial infection. An effective concentration of the surrogate antibody to produce an anti-microbial effect is the concentration that is sufficient to decrease the microbial population by at least about 5%, 10%, 20%, 30%, 40%, 50%, 60%, 70%, 80%, 90%, or higher. Alternatively, the effective dose can be sufficient to decrease the microbial population by 1 log, 2, logs, 3, logs or higher.

Surrogate antibodies having an anti-microbial agent attached thereto can be administered in a therapeutically effective concentration to a host suffering from a microbial infection. Administration may be topical or systemic, depending on the specific microorganisms. Methods for administering the surrogate antibodies of the invention are discussed in more detail below. Generally, the therapeutically effective dose will be sufficient to decrease the microbial population by at least about 10%, 20%, 30%, 40%, 50%, 60%, 70%, 80%, 90%, or higher. Alternatively, the does can be sufficient to decrease the microbial population by 1 log, 2, logs, 3, logs or higher.

Assays to determine the susceptibility of a particular microbe to a surrogate antibody having an anti-microbial agent attached thereto may be determine by in vitro testing. Generally, a culture of microbe is combined with the surrogate antibody having an anti-microbial agent attached thereto at varying concentrations for a period of time sufficient to allow the agent to act. The viable microbes (virus, bacteria and/or fungi) are then counted and the level of killing is determined. One of skill will recognize that culture conditions should be adapted for the specific growth requirements of each organism of interest.

Exemplary assays include the CFU-determination of bacteria and fungi. The CFU-assay for bacteria and fungi has been performed as previously described in Porter et al. (1997) Infect. Immun. 65:2396-2401. Briefly, microorganisms and surrogate antibody having the anti-microbial agent attached thereto are mixed and co-incubated at 37° C. for three hours in the presence of 10 mM PO₄ pH 7.4 with 0.03% Trypticase Soy Broth (TSB, Becton-Dickinson) for bacteria or 0.03% Sabouraud Dextrose Broth (SAB, Difco) for fungi in a final volume of 50 μl. Following incubation the samples are diluted 1: 100 in ice-cold 10 mM PO₄ and spread on Trypticase Soy Agar or Sabouraud Dextrose Agar plates (Clinical Standard Laboratories Rancho Domingez, Calif) with a spiral plater (Spiral Systems, Cincinnati, Ohio.), which delivers a defined volume per area and thus allows precise counts of microbial colonies.

Other assays include radial diffusion. The agar radial diffusion assay has been previously described by Lehrer et al. (1991) J Immunol Methods 137:167-73, herein incorporated by reference. A bacterial-agar layer is prepared by adding 4×10⁶ CFU/ml to 10 ml of a 3% agarose solution with 0.03% TSB. 3 mm wells are punched into the underlay, and 5 μl of the surrogate antibody/anti-microbial agent dilution are allowed to diffuse into the agar for three hours at 37° C. and 10 ml of a 6% TSB 3% agarose is overlaid and plates are incubated overnight. The clear zone diameter in the microbial carpet is measured. See, for example, U.S. Pat. Nos. 6,465,429 and 6,469,137, herein incorporated by reference.

A reduction in the level of active viral particle can be assayed as measured by counting plaque forming units (PFUs). See, for example, Bechtel et al. (1988) Biomat Art Cells Art Org 16:123-128, herein incorporated by reference. Alternatively, a reduction in active viral particles encompasses a decrease in viral titer, as determined by TCID₅₀ values. TCID₅₀ is defined herein as the tissue culture infectious dose resulting in the death of 50% of the cells.

In vivo assays for anti-microbial activity are also known in the art. For example, a test subject can be challenged with the microbe of interest. A therapeutically effective concentration of the surrogate antibody is administered and the delay or inhibition of the microbe population and/or reduction in the microbe population is determined. As such, a therapeutically effective dose can be assayed by determining the reduction in the growth or population of a microbial population or alternatively, the therapeutically effective does can be assayed by an improvement in clinical symptoms of the subject receiving the treatment.

Combined formulations of anti-microbial agents may be used. In one embodiment, the surrogate antibody may have one or more of the same and/or different anti-microbial compounds attached thereto. In other embodiments, multiple surrogate antibodies having the different anti-microbial compounds can be contacted to the microbe population. Alternatively, the surrogate antibody conjugated with the anti-microbial agent may be administered to the microbe population in combination with additional anti-microbial agents.

The methods and compositions of the invention therefore find use in the treatment or prevention of a microbial infection. In this embodiment, by “treatment or prevention” is intended any decrease in the growth of a microbial population in a subjection and/or a decrease in the number of microorganisms contained in the microbe population. Assays to determine this anti-microbial activity are described elsewhere herein.

IV. Pharmaceutical Compositions and Methods of Delivery

The surrogate antibody molecule of the invention may further comprise an inorganic or organic, solid or liquid, pharmaceutically acceptable carrier. The carrier may also contain preservatives, wetting agents, emulsifiers, solubilizing agents, stabilizing agents, buffers, solvents and salts. Compositions may be sterilized and exist as solids, particulates or powders, solutions, suspensions or emulsions.

The surrogate antibody can be formulated according to known methods to prepare pharmaceutically useful compositions, such as by admixture with a pharmaceutically acceptable carrier vehicle. Suitable vehicles and their formulation are described, for example, in Remington's Pharmaceutical Sciences (16th ed., Osol, A. (ed.), Mack, Easton Pa. (1980)). In order to form a pharmaceutically acceptable composition suitable for effective administration, such compositions will contain an effective amount of the surrogate antibody molecule, either alone, or with a suitable amount of carrier vehicle.

The pharmaceutically acceptable carrier will vary depending on the method of administration and the intended method of use. The pharmaceutical carrier employed may be, for example, either a solid, liquid, or time release. Representative solid carriers are lactose, terra alba, sucrose, talc, gelatin, agar, pectin, acacia, magnesium stearate, stearic acid, microcrystalin cellulose, polymer hydrogels, and the like. Typical liquid carriers include syrup, peanut oil, olive oil, cyclodextrin, and the like emulsions. Those skilled in the art are familiar with appropriate carriers for each of the commonly utilized methods of administration. Furthermore, it is recognized that the total amount of surrogate antibody administered will depend on both the pharmaceutical composition being administered (i.e., the carrier being used), the mode of administration, binding activity and the desired effect (i.e., a method of detecting, a method of modulating, or a method of delivering a therapeutic agent).

Once the pharmaceutical composition has been formulated, it may be stored in sterile vials as a solution, suspension, gel, emulsion, solid, or dehydrated or lyophilized powder. Such formulations may be stored either in a ready to use form or requiring reconstitution immediately prior to administration.

The surrogate antibodies also can be delivered locally to the appropriate cells, tissues or organ system by using a catheter or syringe. Other means of delivering such surrogate antibodies oligomers locally to cells include using infusion pumps (for example, from Alza Corporation, Palo Alto, Calif.) or incorporating the surrogate antibodies into polymeric implants (see, for example, Johnson eds. (1987) Drug Delivery Systems (Chichester, England: Ellis Horwood Ltd.), which can affect a sustained release of the therapeutic surrogate antibody to the immediate area of the implant.

A variety of methods are available for delivering a surrogate antibody to a subject (i.e., an animal (mammal), tissue, organ, or cell). The manner of administering surrogate antibodies for systemic delivery may be via subcutaneous, ID, intramuscular, intravenous, or intranasal. In addition inhalant mists, orally active formulations, transdermal iontophoresis or suppositories, are also envisioned. One carrier is physiological saline solution, but it is contemplated that other pharmaceutically acceptable carriers may also be used. In one embodiment, it is envisioned that the carrier and the surrogate antibody molecule constitute a physiologically-compatible, slow release formulation. The primary solvent in such a carrier may be either aqueous or non-aqueous in nature. In addition, the carrier may contain other pharmacologically-acceptable excipients for modifying or maintaining the pH, osmolarity, viscosity, clarity, color, sterility, stability, rate of dissolution, or odor of the formulation. Similarly, the carrier may contain still other pharmacologically-acceptable excipients for modifying or maintaining the stability, rate of dissolution, release, or absorption of the surrogate antibody. Such excipients are those substances usually and customarily employed to formulate dosages for parental administration in either unit dose or multi-dose form.

For example, in general, the disclosed surrogate antibody can be incorporated within or on microparticles or liposomes. Microparticles or liposomes containing the disclosed surrogate antibody can be administered systemically, for example, by intravenous or intraperitoneal administration, in an amount effective for delivery of the disclosed surrogate antibody to targeted cells. Other possible routes include trans-dermal or oral administration, when used in conjunction with appropriate microparticles. Generally, the total amount of the liposome-associated surrogate antibody administered to an individual will be less than the amount of the unassociated surrogate antibody that must be administered for the same desired or intended effect.

By “effective amount” is meant the concentration of a surrogate antibody that is sufficient to elicit a desired effect (i.e., the detection of a ligand, the modulation of ligand activity, or delivering an amount of a therapeutic agent to elicit a desirable effect).

Thus, the concentration of a surrogate antibody in an administered dose unit in accordance with the present invention is effective to produce the desired effect. The effective amount will depend on many factors including, for example, the specific surrogate antibody being used, the desired effect, the responsiveness of the subject, the weight of the subject along with other intrasubject variability, the method of administration, and the formulation used. Methods to determine efficacy, dosage, Ka, and route of administration are known to those skilled in the art.

An embodiment of the present invention provides for the administration of a surrogate antibody in a dose of about 0.5 mg/kg, 1.0 mg/kg, 1.5 mg/kg, 2 mg/kg, 2.5 mg/kg, 3.0 mg/kg, 4.0 mg/kg, 5.0 mg/kg, 6.0 mg/kg, 15.0 mg/kg, 20 mg/kg. Alternatively, the surrogate antibody can be administered in a dose of about 0.2 mg/kg to 1.2 mg/kg, 1.2 mg/kg to 2.0 mg/kg, 2.0 mg/kg to 3.0 mg/kg, 3.0 mg/kg to 4 mg/kg, 4 mg/kg to 6 mg/kg, 6 mg/kg to 8 mg/kg, 8 mg/kg to 15 mg/kg, or 15 mg/kg to 20 mg/kg.

It is recognized that the total amount of surrogate antibody administered as a unit dose to a particular tissue will depend upon the type of pharmaceutical composition being administered, that is whether the composition is in the form of, for example, a solution, a suspension, an emulsion, or a sustained-release formulation. For example, where the pharmaceutical composition comprising a therapeutically effective amount of the surrogate antibody is a sustained-release formulation, the surrogate antibody is administered at a higher concentration.

It should be apparent to a person skilled in the art that variations may be acceptable with respect to the therapeutically effective dose and frequency of the administration of the surrogate antibody in this embodiment of the invention. It is recognized that a single dosage of the surrogate antibody may be administered over the course of several minutes, hours, days, or weeks. A single dose of the surrogate antibody may be sufficient. Alternatively, repeated doses may be given to a patient over the course of several hours, days or weeks. In addition, if desired, a combination of surrogate antibodies may be administered as noted elsewhere herein.

Further, the therapeutically effective amount or dose of a surrogate antibody and the frequency of administration will depend on multiple factors including, for example, the reason for treatment. Some minor degree of experimentation may be required to determine the most effective dose and frequency of dose administration, this being well within the capability of one skilled in the art once apprised of the present disclosure. The method of the present invention may be used with any mammal. Exemplary mammals include, but are not limited to rats, cats, dogs, horses, cows, sheep, pigs, and more preferably humans.

Thus the present invention also provides pharmaceutical formulations or compositions, both for veterinary and for human medical use, which comprise the a surrogate antibody with one or more pharmaceutically acceptable carriers thereof and optionally any other therapeutic ingredients. The carrier(s) must be pharmaceutically acceptable in the sense of being compatible with the other ingredients of the formulation and not unduly deleterious to the recipient thereof.

The compositions include those suitable for oral, rectal, topical, nasal, ophthalmic, or parenteral (including intraperitoneal, intravenous, subcutaneous, or intramuscular injection) administration. The compositions may conveniently be presented in unit dosage form and may be prepared by any of the methods well known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier or both, and then, if necessary, shaping the product into desired formulations.

Compositions of the present invention suitable for oral administration may be presented as discrete units such as capsules, cachets, tablets, lozenges, and the like, each containing a predetermined amount of the active agent as a powder or granules; or a suspension in an aqueous liquor or non-aqueous liquid such as a syrup, an elixir, an emulsion, a draught, and the like.

A syrup may be made by adding the active compound to a concentrated aqueous solution of a sugar, for example sucrose, to which may also be added any accessory ingredient(s). Such accessory ingredients may include flavorings, suitable preservatives, an agent to retard crystallization of the sugar, and an agent to increase the solubility of any other ingredient, such as polyhydric alcohol, for example, glycerol or sorbitol.

Formulations suitable for parental administration conveniently comprise a sterile aqueous preparation of the active compound, which can be isotonic with the blood of the recipient.

Nasal spray formulations comprise purified aqueous solutions of the active agent with preservative agents and isotonic agents. Such formulations are preferably adjusted to a pH and isotonic state compatible with the nasal mucous membranes.

Formulations for rectal administration may be presented as a suppository with a suitable carrier such as cocoa butter, or hydrogenated fats or hydrogenated fatty carboxylic acids.

Ophthalmic formulations are prepared by a similar method to the nasal spray, except that the pH and isotonic factors are preferably adjusted to match that of the eye.

Topical formulations comprise the active compound dissolved or suspended in one or more media such as mineral oil, petroleum, polyhydroxy alcohols or other bases used for topical formulations. The addition of other accessory ingredients as noted above may be desirable.

Further, the present invention provides liposomal formulations of the surrogate antibody. The technology for forming liposomal suspensions is well known in the art. When the surrogate antibody is an aqueous-soluble salt, using conventional liposome technology, the same may be incorporated into lipid vesicles. In such an instance, due to the water solubility of the compound, the compound will be substantially entrained within the hydrophilic center or core of the liposomes. The lipid layer employed may be of any conventional composition and may either contain cholesterol or may be cholesterol-free. When the compound or salt of interest is water-insoluble, again employing conventional liposome formation technology, the salt may be substantially entrained within the hydrophobic lipid bilayer that forms the structure of the liposome. In either instance, the liposomes that are produced may be reduced in size, as through the use of standard sonication and homogenization techniques. The liposomal formulations containing the progesterone metabolite or salts thereof, may be lyophilized to produce a lyophilizate which may be reconstituted with a pharmaceutically acceptable carrier, such as water, to regenerate a liposomal suspension.

Pharmaceutical formulations are also provided which are suitable for administration as an aerosol, by inhalation. These formulations comprise a solution or suspension of the desired surrogate antibody or a plurality of solid particles of the compound or salt. The desired formulation may be placed in a small chamber and nebulized. Nebulization may be accomplished by compressed air or by ultrasonic energy to form a plurality of liquid droplets or solid particles comprising the compounds or salts.

In addition to the aforementioned ingredients, the compositions of the invention may further include one or more accessory ingredient(s) selected from the group consisting of diluents, buffers, flavoring agents, binders, disintegrants, surface active agents, thickeners, lubricants, preservatives (including antioxidants) and the like.

The present invention will be better understood with reference to the following nonlimiting examples.

### EXPERIMENTAL

### EXAMPLE 1

### Process for Making a Ligand-Binding Surrogate Antibody Reagent

An initial library of “Surrogate Antibody” (Sab) molecules was assembled by hybridizing two oligonucleotide strands of pre-defined sequence that were obtained commercially (Life Technologies). Two microliters (100 pmole/microliter) of a 78 nt oligonucleotide strand having the sequence of “(5′) GTA-AAA-CGA-CGG-CCA-GT-Random 40 nt-TCC-TGT-GTG-AAA-TTG-TTA-TCC (3′)” (SEQ ID NO:5) and two microliters (100 pmole/microliter) of a 40 nt oligonucleotide strand having the sequence of “(5′) Biotin-GGT-TAA-CAA-TTT-CAC-ACA-GGA-GGA-CTG-GCC-GTC-GTTTTA-C (3′)” (SEQ ID NO:6) were mixed in a modified Tris buffer, pH 8.0 containing MgSO₄. The solution was heated to 96° C. using a thermal cycler and allowed to hybridize as the solution was cooled to room temperature. SEQ ID NO:5 comprises the specificity strand. The first constant region is underlined and the second constant region has a double underline. SEQ ID NO:6 represents a stabilization region strand. The first stabilization domain is denoted with a single underline. The second stabilization domain is denoted with a double underline.

A library of 1.2×10¹⁴ surrogate antibody molecules was added to 20 μl (1 μg/μl) of a Bovine Serum Albumin (BSA) Polychlorinated Biphenyl (PCB) conjugate suspended in modified Tris buffer, pH 8.0, containing 10% methanol. The solution was incubated for RT/25° C. and transferred to a MICROCON®′-PCR filtration device (Millipore). This filtration device was previously determined to retain SAb molecules bound to the BSA-PCB conjugate and not retain unbound SAb molecules. SAb bound to the conjugate was separated from unbound molecules by centrifuging the incubation solution at 1000 g/10′/RT. The BSA-PCB bound SAb in the retentate was washed three times with 200 μl aliquots of the modified Tris buffer.

SAb in the washed retentate was aspirated (˜4011) from the filter and transferred into a PCR Eppendorf tube. The recovered SAb-BSA-PCB complex was used to amplify the 78 nt strand without prior dissociation from the conjugate. DNA polymerase, nucleotide triphosphates (NTP), buffer, and an M13R48 primer specific for the starting positive strand and having the sequence (5′) Biotin-GGA-TAA-CAA-TTT-CAC-ACA-GGA (3′) (SEQ ID NO:7) was used in the polymerase chain reaction (PCR) to first produce an amplified population of 78 nt negative strands (i.e., specificity strand). A thermal cycler was programmed to perform 40 cycles of amplification at temperatures of 96° C., 48° C., and 72° C. for 30-300″.

An amplified population of the positive 78 nt strand was next produced from the amplified 78 nt negative strand material using asymmetric PCR. Approximately 5% of the amplified 78 nt negative strand was added to an Eppendorf PCR tube with 40 μl of DI H₂O. Polymerase, NTP, buffer, and an M13-20 primer specific for the negative strand and having the sequence (5′) Biotin-GTA-AAA-CGA-CGG-CCA-GT (3′) (SEQ ID NO:8) was added and used for PCR amplification. The temperature cycles previously cited were again used. Less than 4% of the amplified population was found to contain either 78 nt negative or 40 nt positive strands. Purification to remove polymerase, NTP, primer and 40 nt oligomers was performed using a commercial product (Qiagen PCR Purification Kit).

Re-assembly of the 120 nt, double-stranded, SAb was performed by hybridizing the captured, amplified, and purified 78 nt strand (i.e., specificity strand) with the 40 nt starting oligonucleotide (i.e., stabilization strand). This reassembly process produces an enriched library of ligand-binding SAb molecules. Enriched SAb libraries are assembled prior to beginning each of the subsequent rounds of selection. These subsequent cycles use a positive selection process to enhance the average specificity and affinity of the SAb population for the target ligand.

Approximately 80% (40 μl) of the purified 78 nt material was added to a 200 μl Eppendorf tube containing modified Tris buffer and 5 μl (10 pmole/ul) of the 40 nt strand. Deionized water (35 μl) was added and the mixture heated to 96° C./5′, 65° C./5′, 60° C./5′, and 56° C./5′. The solution was then allowed to cool at the rate of 1° C./min. for 30′ until it reached RT. The solution was filtered through a Microcon® filtration device (5′/1000 g/RT) and the filtrate was collected for use in a subsequent cycle of selection.

Several capture and amplification selection cycles (i.e. 2-6), each preceded by the amplification of the 78 nt oligonucleotide strand, purification, and SAb assembly, were used to produce an enriched library of BSA-PCB-binding SAb molecules. After completing the capture and amplification cycles, the enriched SAb library was processed to capture and amplify SAb molecules that are specific for the target ligand.

Cycles of specificity selections are used to eliminate SAb molecules in the population that bind carrier proteins, derivative chemistries, or cross-reacting compounds. It results in the production of an enriched SAb population of molecules that specifically bind the target ligand. When producing a SAb population that can specifically bind unique determinants on neoplastic tissue, specificity selections eliminate SAb molecules that bind to normal cell constituents.

The process of separating bound from unbound SAb using the MICROCON® filtration device was used as previously explained. The enriched SAb library produced during the capture and amplification phase was incubated with a solution of unconjugated Bovine Serum Albumin (20 μg/ml) for 60′/RT. The solution was then filtered through a MICROCON® filtration device (5′/1000 g/RT). The filter retains SAb bound to BSA. SAb in the filtrate was recovered and used to amplify the 78 nt strand and assemble and purify a new SAb library. SAb was incubated with solutions containing untargeted PCB congeners (e.g. BZ54, BZ18, etc.), dioxins, polyaromatic hydrocarbons (e.g. naphthalene, phenanthrene) and other irrelevant haptens prior to incubation with the target PCB (BZ101)-BSA conjugate. The incubated solutions containing the SAb, irrelevant ligand(s), and target conjugate are filtered through the MICROCON® filtration device. Non-specific SAb molecules bound to the cross-reacting ligands in solution are not excluded by the porosity of the filter and pass into the filtrate and are discarded. Molecules bound to the PCB-BSA conjugate, after exposure to potential cross-reacting compounds, are retained by the membrane and are processed into a new SAb population. These molecules are used to amplify the 78 nt strand and assemble a specific population of SAb molecules that are then used in cycles of sensitivity selections to capture the highest binding affinity molecules.

Cycles of sensitivity selections are used to capture the highest affinity SAb molecules from a library of specific binding molecules for the purpose of preparing a specific, high affinity, polyclonal SAb library. The process exposes the SAb library produced after cycles of specificity selections to reduced concentrations of the target ligand and agents and conditions that compromise hydrophobic, electrostatic, hydrogen, Van der Waals binding interactions. Such agents and conditions include solvents (e.g. methanol), pH modifications, chaotropic agents (e.g. guanidine hydrochloride), elevated salt concentrations, surfactants (e.g. tween, triton) that can be used alone or in combination. The process compromises ligand binding and selects for the highest binding affinity molecules. Once selected these molecules are used as a template to amplify the 78 nt strand and assemble an enriched polyclonal population.

Sensitivity selections are performed using the enriched SAb population obtained after completing the “capture and amplification” and “specificity selections”. The solution-phase process of capturing, or eliminating, SAb on the basis of their binding to a ligand and capture using a molecular sieving filtration device was again used. The SAb was incubated with unconjugated PCB molecules prior to the addition of the BSA-PCB (BZ101) conjugate for 60′/RT. The incubation solution was introduced into a MICROCON® filtration device and centrifuged at 1000 g/10′/RT. SAb bound to the unconjugated PCB molecules proceed into the filtrate where they are collected and used to amplify the 78 nt strand and assemble an enriched population of molecules that bind the unconjugated ligand. The enriched population was incubated with the PCB-BSA conjugate at a reduced concentration (0.4 μg/ml) and SAb bound to the conjugate are recovered after filtration using the MICROCON® device (1000 g/10′/RT) and washing three times using a modified Tris buffer containing 0.05% Tween 20. Recovered SAb in the retentate was amplified to produce 78 nt strands and assembled into SAb molecules. The process was repeated by incubating the SAb library with the PCB-BSA (0.4%) conjugate in the presence of methanol (10% v/v) and Tween 20 (0.05%). SAb bound to the conjugate was recovered in the retentate and used to amplify the 78 nt strand. A polyclonal SAb population was assembled as described above. The polyclonal SAb population can be fractionated into individual monoclonal SAb reagents using the following procedures.

### EXAMPLE 2

### Monoclonal SAb Preparation

The polyclonal SAb population is amplified by PCR to produce double stranded 78 nt and double stranded 40 nt molecules using specific primers. Amplification artifacts and PCR-errors are minimized by using polymerase with high fidelity and low number PCR cycles 1(25 cycles). PCR products are elctrophoresized in 3½ high resolution agarose gel and 78 nucleotide fragments are recovered and purified by Qiagen Gel extraction kid. The purified 78 nt double strand DNA are cloned into PCR cloning vector (such as pGEM-T-Easy) to produce plasmid containing individual copies of the ds 78 nt fragment. The E. coli bacteria (e.g. strain JM109, Promega) are transformed with the plasmids by electroporation.

The transformed bacteria are cultured on LB/agar plates containing 100 μg/ml Ampicillin. Bacteria containing the 78 nt fragment produce white colonies and bacteria that do not contain the 78 nt fragment expresses 13 gal and form blue colonies. Individual white colonies are transferred into liquid growth media in microwells (e.g. SOC media, Promega) and incubated overnight at 37° C.

The contents of the wells are amplified after transferring an aliquot from each well into a PCR microplate. The need to purify the PCR product is avoided by using appropriate primer and PCR conditions. SAb molecules are assembled in microplates using the previously cited process of adding 40 nt-fragments and hybridization in a thermalcycler using a defined heating and cooling cycle.

### EXAMPLE 3

### Analysis and Database Construction

Reactive panel profiling of monoclonal SAb clones is used to compare binding characteristics used in selecting reagent(s) for commercial application. Characteristics that are analyzed can include:

1) recognition of target ligand;

2) relative titer and affinity;

3) sensitivity;

4) specificity;

5) matrix effects;

6) temperature effects;

7) stability; and

8) other variables of commercial significance (e.g., lysis, effector function).

Standard test protocols are used and data collected from each clone is entered into a relational database.

Characterization assays transfer aliquots of assembled monoclonal SAb reagents to specific characterization plates for analysis. Affinity and titration assays compare relative affinity (Ka) and concentration of each reagent. Sensitivity assays compare the ability to detect low concentrations of the target ligand and provide an estimate of Least Detectable Dose. Specificity assays compare SAb recognition of irrelevant/undesirable ligands. Matrix interference studies evaluate the effect of anticipated matrix constituents on the binding of SAb. Temperature effects evaluate the relationship to binding. Stability identifies the most stable clones and problems requiring further evaluation. Other characteristics relevant to the anticipated application can also be evaluated using known means.

Target ligands for SAb binding include prokaryotic cells (e.g. bacteria), viruses, eukaryotic cells (e.g. epithelial cells, muscle cells, nerve cells, sensory cells, secretory cells, malignant cells, erythroid and lymphoid cells, stem cells, protozoa, fungi), proteins, prions, nucleic acids, and conjugated filterable compounds. The target ligands for SAb binding can be any ligand of sufficient size that can be retained by a filter membrane/molecular sieve.

### EXAMPLE 4

### Preparation of Surrogate Antibody 87/48 to PCB congener BZ101 Using Non-Amplifiable Stabilization Strand

Surrogate Antibody (SAb) molecules were produced using self-assembling oligonucleotide strands (87 nt+48 nt) to form a dimeric molecule having a 40 nt random specificity domain sequence with adjacent constant nucleotide sequences. Cycles of ligand binding, PCR amplification, bound/free separation, and reassembly/reannealing were used to enrich the SAb population with molecules that would bind a BSA-Adipoyl-BZ101 conjugate and the unconjugated BZ101 (2,2′,4,5,5′ pentachlorobiphenyl) hapten.

Methods

A. Forming a Library of Surrogate Antibodies:

A library of 87 nt ssDNA oligonucleotides containing a random 40 nt sequence, and FITC (F) and biotinylated (B) primers, were purchased from IDT. The 87 nt ssDNA was designated #22-40-25 (87 g2) to reflect the numbers of nucleotides in the constant sequence regions flanking the variable region. The is the specificity strand of the surrogate antibody molecule and the sequence of the 87 mer is shown below (top strand; SEQ ID NO: 9), while the 48 nt oligonucleotide (stabilization strand) shown is below (bottom strand; SEQ ID NO: 10).

5′- GTA AAA CGA CGG CCA GTG TCT C - (40 nt) - A GAT TCC TCT GTG AAA TTG TTA TCC -3′
||| ||| ||| ||| ||| || ||| ||| ||| ||| ||| |||
3′- CAT TTT GCT GCC GGT CA ggagctctcg AGG ACA CAC TTT AAC AAT AGG- 5′

The two constant region nucleotide sequences on either side of the variable sequence are complementary to the nucleotide sequences of a juxtaposed 48 nt. stabilization oligonucleotide. The stabilization strand is FITC-labeled 5′- and referenced as oligonucleotide (#F21-10-17) (bases in bold are non-complimentary to bases on the 87 nt specificity strand):

Oligos were reconstituted in DI water to 0.1 mM (100 pm/μl) and stored as stock solutions in 2 ml screw top vials at −20° C. (manufacturer claim for reconstituted stability is >6 months). Working aliquots of 20 μl each were dispensed into PCR reaction tubes and stored at −20° C.

B. Selection; Cycle 1

4 μl of 0.1 mM ssDNA oligonucleotide A22-40-25 (i.e. “+87”) library (2.4×10¹⁴ molecules) were mixed with 4 μl of 0.1 mM F21-10-17 (i.e. “−40”) that is FITC-labeled at 5′ end and 2 μl of 5×TNKMg5 (i.e. TNK buffer containing 5 mM MgSO4) buffer. TNK Buffer is a Tris Buffered Saline, pH 8.0. The 5×stock comprise 250 mM Tris HCl, 690 mM NaCl, 13.5 mM KCl and a working (1×) buffer comprises 50 mM Tris HCl, 138 mM NaCl, and 2.7 mM KCl. TNK5Mg is TNK above with 5 mM MgSO₄ (1:200 dilution of 1M MgSO₄ stock) and 5×TNK5Mg is 5×TNK with 25 mM MgSO4 (1:40 dilution of 1M MgSO₄).

Annealing of SAb molecules was performed using the HYBAID PCR EXPRESS thermal cycler. The oligo mixture was heated to 96° C. for 5′, the temperature was reduced to 65° C. at a rate of 2° C./sec and maintained at this temperature for 20 min. The temperature was then reduced to 63° C. at 2° C./sec and maintained at this temperature for 3 min. The temperature was then reduced to 60° C. at 2° C./sec and maintained at this temperature for 3 minutes. The temperature was then reduced in 3° C. steps at 2° C./sec and held at each temperature for 3 minutes until the temperature reaches 20° C. Total time from 60° C. to 20° C. is 40 min. Total annealing time of 1.5 hours.

To assay for the formation of the surrogate antibody eletrophoresis was employed. On each preparative gel, a FAM-87 and F-48 was loaded to demonstrate the location of the corresponding bands and SAb. On a parallel gel (or the other half of the preparative gel), a 10 bp ladder, 48 ss, 87 ss and the retentate PCR product next to an aliquot (0.5 μl) of each annealed SAb. 10 μl of reaction mixture from above was mixed with 7 μl, 60% w/v sucrose. Mixture was loaded onto a 20% acrylamide gel. The 48 nt (F21-10-17) and dsSAb appeared as green fluorescent bands. The 48 band runs at approximately 50 base pairs and the dsSAb runs about 304. After extracting the Sab, the gel is stained with EtBr (1 μl of 10 mg/ml into 10 ml buffer). The 87 band will appear at approximately 157 bp, using the standard molecular weight function.

The gel fragment containing the SAB 87/48 band was excised and place in a 1.5 ml eppendorf tube. The gel fraction was macerated using a sterile pipette tip and 400 μl TNKMg5 buffer containing 0.05% v/v Tween 20 is added and the sample is then shaken on a rotating platform at the lowest speed for 2 hours/RT. The gel slurry was aspirated and added to a Pall Filter 300K and spun in Eppendorf 5417R at 1-5000×g (7000 rpm) for 3′. 40 μl TNKMg5 buffer containing 0.05% Tween was added to a volume <440 μl and centrifuge 3′.

The volume of filtrate is measured. RFU (relative fluorescence units) of the formed Sab was measured using a 10 μl aliquot of the filtrate and 90 μl buffer, and the Wallac VICTOR2, mdl 1420 (Program name “Fluorescein (485 nm/535 nm, 1”). A blank of buffer only was also measured. Total fluorescence was calculated by subtracting the background and multiplying by the appropriate dilution factor and volume. 1/10 volume (40 μl) MeOH was added to the filtrate along with 20 μl BSA-aa-BZ101 conjugate (1 μg/μl conjugate concentration in TNKMg5 Tw0.05 containing 10% MeOH v/v) to filtrate. The BSA-AA-BZ101 conjugate, synthesis, characterization was performed as outlined in Example 5. The sample was incubated for 1 hour/RT.

The reaction mixture was aspirated and added to a new Nanosep 100K Centrifugal Device and centrifuge at 1000 g/3′. (The Nanosep 100K and 300K Centrifugal Devices were pruchaced form PALL-Gelman Cat #OD100C33 and are centrifugal filters with Omega low protein and DNA binding, modified polyethersulfone on polyethylene substrate.) The filters were used to fractionate SAb bound to BSA-AD-BZ101 from unbound Sab. SAb bound to the conjugate was recovered in the retentate while unbound SAb continued into the filtrate. The filtrate was aspirated and added to new 1.5 ml Eppendorf tube. 100 μl of mixture was removed and the RFU's was quantified in a microwell plate using Wallac Victor II. The retentate was washed only one time for cycle 1 (two times for cycle 2 and 3 times for cycles 3-6) at 1000 g/3-8′ using 400 μl aliquots of TNKMg5 buffer (without Tween and MeOH). Spin times vary from filter to filter (generally 3-8 minutes). Retentate was saved for SAb, keep filtrate and pool to measure fluorescence x volume to coincide with retentate RFU. Filtrate was discarded.

SAb (when SAb is bound to conjugate, MW>100 KD) in the retentate was recovered by adding a 100 μl aliquot of DI H₂O, swirling, and aspirating. The Total RFU's was calculated for the recovered material. Percent recovery was calculated by calculating total recovered vs. total in starting amount of SAb incubated with conjugate.

B. PCR Amplification

The DNA recovered from the retentate was amplified using a 40 cycle PCR amplification program and 2 μM of primer F22-5 and 2 μM of primer Bio21-4. Bio21-4 adds biotin to 5′ end of −87 oligonucleotide.

PCR Primers. The primers were designed to amplify only the 87 strand (the specificity strand) and not the 48 strand (the stabilization strand). This was accomplished by having 4-5 bases on the 3′ end that compliment the 87 strand but not the 48 strand. See FIG. 7. Four to five bases of non-complimentarity was sufficient to inhibit elongation.

The primer sequences used for PCR amplification were as follows. Primer F22-5—amplifies off of the −87 strand to make a new +87 and comprise the sequence: 5′ FAM-GTA AAA CGA CGG CCA GTG TCT C₃′(SEQ ID NO: 11). Primer Bio-21-4—amplifies off of the +87 to make a biotin-labeled −87 that in some embodiments can be used to extract −87 strands that do not anneal to the 48. The sequence for Bio-21-4 is 5′ bio-GGA TAA CAA TTT CAC ACA GGA ATC T 3′ (SEQ ID NO: 12).

Primers were reconstituted in 10 mM Tris (EB) to 0.1 mM (100 pm/μl) and stored in 2 ml screw top vial at 20° C. as a stock solution (claim for reconstituted stability is >6 months). Working aliquots of 20 μl were dispensed into PCR reaction tubes and stored frozen at −20° C.

PCR reaction: 10 μl of the retentate was added to a 0.2 ml PCR tube. 5 μl of Thermopol 10× buffer, 1 μl NTP stock solution (PCR dNTP, nucleotide triphosphates 10 mM (Invitrogen 18427.013) which contains a mixture of 10 mM of each of four nucleotides (A, G, C, T), 12 μL of 5M Betaine (Sigma B-0300) and 10 μl of 10 pmole/μl of each primer was added. QS to 49.5 μl with DI H₂O. The program was run with the following parameters: 3 min, 94°-65°-72° 30 sec each ×35, 10° hold. When PCR machine is at 96° 5 μl of Taq DNA Polymerase ((NEBiolabs cat# M0267S) 5 U/μL) is added the reaction is mixed and placed in PCR machine.

Following the PCR reaction, 5 μL of PCR product were run on a 3% Agarose 1000 gel or 4% E-gel with controls of 10 bp ladder and ss oligos to verify amplification and size of bands. The remaining amplified DNA is purified by salt precipitation using 100% ethanol. Specifically, ⅓ volume (100 μl) of 8M Ammonium Acetate is added to 200 μl of the amplified DNA. 2.6 times the combined (DNA+Ammonium Acetate) volume (780-800 ul) of cold absolute ethanol (−20° C.) is added to the tube. The tube is swirled and stored on ice for 1 hr. The sample is centrifuged for 15′/14,000 g 4° C. in a refrigerated centrifuge. The supernatant liquid is removed without touching or destroying the pellet. 0.5 ml of 70% (V/V) ethanol is added. The sample is mixed gently and centrifuged for 5′/14,000 g. The supernatant is removed without disturbing the pellet and evaporate to dryness by exposing to air at RT.

When amplifying selected DNA from retentate, the following controls are also run: no DNA, 87 alone, and 48 alone. This will assure that the bands from the retentate are the right size and are not due to primer dimers. It will also show that the 48 strand is not amplifying in the SAb tube. By itself, the −48 will amplify and can be detected in the 48 control tube. This will identify the position of the ds 48 in the SAb tube if it was amplified.

Reannealing: The pellet was reconstituted by adding 8 μl of a solution containing 4 μl of sterile DI H₂O+4 μl of 0.1 mM-48 nt oligonucleotide (F21-10-17). The sample was transferred to a 0.2 ml PCR tube and 2 μl of 5×TNKMg5 buffer was added. (Note; the addition of excess F21-10-17 (−48 nt) primer drives the formation of the desired +87/-48 SAb molecules).

B. Cycle 2-6: Annealing SAb

The dsSAb was annealed by heating the reconstituted material in a 0.2 ml PCR tube using the temperature program previously specified for annealing. After the first cycle, multiple bands appear. Thus a parallel SAb aliquot was run with its corresponding PCR starting strands to verify that the band being cut out is in fact the new SAb. To verify that the SAb band was ds 87/48, an aliquot was removed and run on a denaturing gel (16%, boiling in 2× urea sample buffer) to verify that the band from the preparative gel contains both 87 and 48 strands.

Electrophoresis was performed at 120 v for 40 min. 7 μl of 60% w/v sucrose was mixed with 10 μl of DNA and the sample is loaded. Any DNA component with FITC at 5′ end (i.e. SAb 87/48, ds 48 and ss48) will appear on the gel as a green fluorescent band under long wavelength. Run 5 pMol of F21-10-17 (−48 nt primer) in an available lane as a size marker. SAb will be observed to co-migrate with 250-300 nt dsDNA in 20% acrylamide native gel. The SAb-gel section was excised and macerated in 250 μl of TNKMg5 Tw0.05 buffer. The sample was a incubated for 2 hrs/RT while agitating on rotating platform at the lowest speed.

The gel suspension was transferred to a Pall 300K Centrifugal Device and centrifuge at 1-5000 g/3′ to remove the polyacrylamide. The retentate was washed by adding a 50 μl aliquot of buffer, centrifuge at 1000 g/3′. The SAb is recovered from the filtrate for use in subsequent selection cycle.

The RFU's of SAb and buffer blank was measured as describe above using a 100 ul aliquot of the filtrate on the Wallac Victor2.

C. Selection Cycles 2-7

1/10 volume of MeOH was added and 20 μl BZ101-aa-BSA (1 μg/μl) as in cycle 1. The sample was incubated for 1 hr and selected using Pall 100K filter. RFU measurement of the retentate after 2 washes for cycle 2 and 3 washes for cycle 3-6 were taken. Subtraction of the background RFU allow the determination of the % recovery.

Negative Selection. In this example, negative selection using BSA was not performed in Cycle #1-6.

When negative selection was desired, 250 μl of SAb 87/48 filtrate (2-20 pMol by FITC) was mixed with 20 μl of a 1 μg/μl (20 μg) BSA solution. The sample is incubated for 30′/RT. The RFU's was measured in 100 ul aliquot using Wallac VICTOR II Program.

250 ul of the above reaction mix (20 μl is saved for 16% non-denaturing PAGE and 8% denaturing PAGE with 8M urea) was added to Nanosep 100K Centrifugal concentrator. The filter was centrifuged at 1000 g/15′/RT. Total volume in filtrate was ˜240 μl. Aspirate filtrate and place in new 1.5 ml Eppendorf tube. RFU's of 100 μl aliquot were checked.

The filter was washed by adding 200 μl TNKMg5 buffer, centrifuge (1000 g/10′/RT), add additional 200 μl of same buffer after centrifugation, re-centrifuge, add 100 μl of same buffer and centrifuge again. 100 μl DI H₂O was added, filtered, swirled and aspirate retentate. RFU's were determined on Wallac VICTOR II of SAb bound to BSA by aspirating retentate and % recovery was determined. 200 μl of negatively selected filtrate was mixed with 20 μl (1 μg/μl) of the BSA-aa-BZ10 conjugate suspended in TNKMg5 buffer. The mixture was incubated for 1 hour/RT with a total volume of 220 μl. The reaction solution was added to a new Nanosep 100K centrifugal device and centrifuged at 1000 g/3′. A wash was performed 3 times using a TNKMg5 buffer. Measure RFU's of a 100 μl aliquot of the filtrate to determine % of unbound (free) SAb.

100 μl of DI H₂O was added to filter, swirled, and the retentate was aspirated. The entire sample was placed in a microtiter plate well. RFU's of sample were measured and background and calculate % Recovery.

Additional Steps. 1-20% of the bound SAb recovered in the 100 μl aliquot was used for PCR amplification with primer. This will again generate dsDNA in 4 tubes each containing 50 μl, as described previously. Cycles of negative and positive selection were repeated until no further enrichment in % recovery was observed in the SAb population.

Additional cycles can be performed by preincubating the free hapten with the polyclonal SAb library prior to addition of the conjugate, and collecting the filtrate for subsequent amplification. A cycle(s) of affinity enhancement can be performed by incubating the SAb and conjugate in the presence of elevated MeOH, surfactant, decreased pH, and/or increased salt. High affinity SAb remaining bound to the conjugate is amplified. The process of Polyclonal SAb production proceeds through 1. Binding, 2. Specificity Enhancement, 3. Affinity Enhancement, prior to production of monoclonal SAb clones.

Calculations. The total amount of RFU's in the recovered conjugate-binding aliquot vs. the total amount of RFU's that were present when incubated with the conjugate was determined. For negative selection; the amount of RFU's in the recovered BSA-binding aliquot vs. the total amount of RFUs present when incubated with BSA was determined. RFUs quantified from filtrate provides supportive data and information indicating unbound SAb and loss on filter device.

Notes: The DNA/conjugate and DNA/BSA ratios in cycles #2-5 was 10-100 nM DNA/2,000 nM protein, or 1 molecule of SAb to 20-200 molecules of the conjugate or BSA. This calculation assumes that the conjugate has the reported 20 moles of BZ101 per mole of protein). The molecular weight of the (SAb 87/48-BSA-aa-BZ111) complex=(A22-40-25=27.4 Kd)+(FM21-10-17=15.4 Kd)+(BSA=67 Kd)+(20 BZ101=7 Kd). Total=116.8 Kd; 2SAb:1 Conjugate=˜159.6 Kd.

### EXAMPLE 5

### Preparation of Surrogate Antibody 78/48 to PCB congener BZ101

Surrogate Antibody (SAb) molecules were produced using self-assembling oligonucleotide strands (78 nt+48 nt) to form a dimeric surrogate antibody molecule having a 40 nt random sequence binding loop with adjacent constant nucleotide sequences. Cycles of ligand binding, PCR amplification, bound/free separation, and reassembly/reannealing were used to enrich the SAb population with molecules that would bind a BSA-Adipoyl-BZ11 conjugate and the unconjugated BZ1O1 (2,2′,4,5,5′ pentachlorobiphenyl) hapten.

A. Background

PCBs are chlorinated aromatic compounds that can exist in 209 different molecular configurations (congeners). The higher chlorinated species are relatively stable to oxidation at elevated temperatures, and were used as heat transfer agents from 1929 to 1977. During this period 1.4 billion pounds were produced and commercialized as mixed congener Aroclor® products, named to reflect their 12 carbon biphenyl nucleus and average percentage of chlorine (e.g. Aroclor 1242, 1248, 1254, etc.). Today these compounds are ubiquitous environmental contaminants, having been used in transformers, industrial machinery and household appliance capacitors, compressors, paint, insulation, adhesives, and chemical processing equipment. The Toxic Substances Control Act (TSCA) of 1976 established the legal framework for their elimination, but prior pollution, new spills, and the continuing disposal of contaminated materials persist. PCBs have been classified as Persistent Organic Pollutants (POPs) and efforts are underway to draft an international treaty that would coordinate their elimination.

Polychlorinated biphenyls (PCBs) have been classified as endocrine disrupters. They mimic estrogens (xenoestrogens) and upset endocrine hormone balance. Male sexual development is dependent upon androgens, and imbalances in the androgen/estrogen ratio caused by PCBs are thought to interfere with genital development. PCBs are linked to neuro-developmental defects in utero and concern exists regarding fetal health in mothers that consume PCB-contaminated fish. PCBs have also been found in breast milk, a significant source of exposure for neonates. Studies have shown that pre-natal exposure to PCBs causes mental and physical abnormalities. Other effects are lower birthing weight, altered thyroid and immune function, and adverse neurological effects. Other studies suggest that persistent exposure of newborns to PCBs results in hypoandrogenic function in adult males (Kim et al. (2001) Tissue Cell 33:169-77).

A health effect of particular concern is the neurotoxicity caused by PCB-altered thyroid function during the critical period of thyroid-dependent brain development. This period extends from pre-partum to 2 years post-partum. Thyroid function regulates the assembly and stability of the cytoskeletal system required for neuronal growth, and the development of the cholinergic and dopaminergic systems of the cerebral cortex and hippocampus. Exposure to PCBs causes enlargement of the thyroid with an accompanying reduction in circulating thyroxine (T4) levels. The likely cause is the structural similarity that exists between selected congeners and the thyroid-hormone, and the ability of PCBs to be bound by transport proteins such as transthyretin with high affinity. PCBs have been shown to act as agonists and antagonists when bound to thyroid receptors. The neurological effects resulting from thyroid disorders, and those reported following PCB or dioxin exposure, bear a striking similarity and suggest a common mechanism.

Three congeners (BZ138, 153, 180) listed in the EPA reference method, interfere with sexual hormone regulation by competing with the natural ligand for binding to two nuclear receptors. These congeners also have different affinities for estrogen and androgen receptors and can induce both cell proliferation (nM) and inhibition (FM). PCBs are suspected agents in the development of endometriosis, have been shown to be immunosuppressive, and can be carcinogenic, Carcinogenesis is believed to be mediated through binding to the Ah receptor (aryl hydrocarbon) via the same pathway described by Poland and others for dioxins.

The surrogate molecules of the invention being developed for the PCB array combine attributes of aptamers and natural antibodies. These molecules are of nucleic acid composition and retain a stable secondary structure having constant regions and a hydrophobic binding cavity. Pre-formed and sequentially enriched libraries of molecules having a random assortment of binding-cavity sequences are fractionated to amplify those that bind the target. A monoclonal antibody procedure will produce homogenous molecules for characterization, identification, sequencing and synthesis. The preparation process is expected to significantly reduce the time of development. The molecule has been designed to permit the simple attachment of multiple labels. Animals are not used, and induction of an immune response is not required. Production is by PCR or direct synthesis. The surrogate antibody molecules facilitate the elimination of PCBs from the environment and remove a persistent public health pathogen.

B. Materials and Methods

I. Selection: Cycle I

Forming the surrogate antibody: The library of surrogate anibodies used in the following experiment was formed as follows. A library of 78 nt ssDNA oligonucleotides containing a random 40 nt sequence, and FITC (F) and biotinylated (B) primers, were purchased from Gibco-Invitrogen life technologies. The 78 nt ssDNA was designated #17-40-21 to reflect the numbers of nucleotides in the constant sequence regions flanking the variable region. The sequence of the 78 mer (i.e., the specificity strand; SEQ ID NO: 13) is shown below along with the 48 nt oligonucleotide (i.e., the stabilization strand; SEQ ID NO: 14).

(78 nt oligonucleotide. shown as top strand)
5′ GTA AAA CGA CGG CCA GT - (40 nt) - TCC TGT GTG AAA TTG TTA TCC 3′
||| ||| ||| ||| ||| || ||| ||| ||| ||| ||| ||| |||
3′CAT TTT GCT GCC GGT CA ggagctctcg AGG ACA CAC TTT AAC AAT AGGF5′
(48 nt oligonucleotide shown as bottom strand)

The two constant region nucleotide sequences on either side of the variable sequence are complementary to the nucleotide sequences of a juxtaposed 48 nt stabilization oligonucleotide. The bases in bold of the FITC-labeled 5′-oligonucleotide (#F21-10-17) are non-complimentary to bases on the 78 nt strand. Oligos were reconstituted in DI water to 0.1 mM (100 pm/μl) and stored as stock solutions in 2 ml screw top vials at −20° C.

4 μl of 0.1 mM ssDNA oligonucleotide A17-40-21 (i.e. “+78”) library (2.4×10¹⁴ molecules) (i.e., specificity strand) was mixed with 4 μl of 0.1 mM F21-10-17,(i.e. “−40”) (stabilization strand) that is FITC-labeled at 5′ end and 2 μl of 5× TNKMg5 (i.e. TNK buffer containing 5 mM MgSO4) buffer. TNK Buffer is Tris Buffered Saline, pH 8.0 (a 1× stock comprises 50 mM Tris HCl 138 mM NaCl and 2.7 mM KCl). The TNKMg5 buffer comprises the TNK buffer plus 5 mM MgSO₄.

SAb molecules were annealed using the HYBAID PCR EXPRESS thermal cycler (program name: “Primer”). The oligo mixture is heated to 96° C. for 5′, the temperature is reduced to 65° C. at a rate of 2° C./sec and maintained at this temperature for 20 min. The temperature was then reduced to 63° C. at 2° C./sec and maintained at this temperature for 3 min. The temperature was then reduced to 60° C. at 2° C./sec and maintained at this temperature for 3 minutes. The temperature was then reduced in 3° C. steps at 2° C./sec and held at each temperature for 3 minutes until the temperature reaches 20° C. Total time from 60° C. to 20° C. is 40 min.

10 μl of reaction mixture from above was mixed with 7 μl, 60% w/v sucrose and loaded onto a 1 mm 16% acrylamide gel (19:1 ratio Acrylamide:Methylene Bisacylamide). The gel was examined using long wave UV-366 nm BLAK-RAY LAMP model UVL-56. The 40 nt (F21-10-17) and dsSAb appear as green fluorescent bands.

The “SAb 78/48” band was excised from the gel and the gel fraction was mascerated in 400 μl TNKMg5 buffer containing 0.05% v/v Tween 20. The gel slice was then shook on a vortex at the lowest speed for 2 hours/RT.

The gel slurry was aspirated and the gel suspension is added to an Amicon (Microcon) Centrifugal Device and spin at 1000 g/10′. 40 μl TNKMg5 buffer containing 0.05% Tween was added and the sample was centrifuge at 1000 g/10′. Total volume ≦440 μl.

40 μl MeOH was added to the filtrate. To quantify the amount of antibody, RFU (relative fluorescence units) was measured using a 100 μl aliquot of the filtrate and the Wallac VICTOR2, mdl 1420 (Program name “Fluorocein (485 nm/535 nm, 1”).

All of the SAb filtrate was added to the Nanosep 100K Centrifugal Device (Pall-Gelman) and it was Centrifuge at 1000 g/15′. RFU was quantified using a 100 μl aliquot of the filtrate as above.

II. Selection of Surrogate Antibody

The filtrate from above is added to a 0.2 ml PCR tube containing 20 μl BSA-aa-BZ101 conjugate (1 μg/μl conjugate concentration) in TNKMg5 Tw 0.05 containing 10% MeOH v/v). BSA-AA-BZ101 conjugate was synthesized as described below. Methanol added to 10% v/v final concentration. Tween 20 was added to 0.05% w/v final concentration. The sample was incubated for 1 hour/RT.

The reaction mixture was aspirated and added to new Nanosep 100K Centrifugal Device and centrifuge at 1000 g/10′. The Nanosep 100K Centrifugal Devices (Cat #OD100C33 PALL-Gelman, centrifugal filter with Omega low protein and DNA binding, modified polyethersulfone on polyethylene substrate) used was able to fractionate SAb bound to BSA-AD-BZ101 from unbound SAb. SAb bound to the conjugate was recovered in the retentate while unbound SAb continued into the filtrate. The filtrate was aspirated and added to new 1.5 ml Eppindorf tube. 100 μl was taken and the RFU's were quantified in a microwell plate using Wallac Victor II. The retentate was washed 3 times at 1000 g/10′ using 200 μl aliquots of TNKMg5 buffer (sans tween and MeOH). The filtrate was discarded.

SAb (when SAb is bound to conjugate, MW>100KD) in the retentate was recovered by adding a 100 μl aliquot of DI H₂O, swirling, and apirating. The Total RFU's was calculated for the recovered material. % recovery was determined by calculating total recovered vs. total in starting amount of SAb incubated with conjugate.

III. PCR Amplification

The DNA recovered from the retentate was amplified using a 40 cycle PCR amplification program and 2 μM of primer FMT3-20 and 2 uM of primer BioM13R48. BioM13R48 adds biotin to the 5′ end of +78 oligonucleotide. The PCR reaction amplifies +78 nt, −48 nt, −78 nt and +48 nt strands thereby reducing the theoretical yield of SAb

The primer sequences used for the PCR amplification are as follows: Primer #FM13-20 (SEQ ID NO: 15) has the sequence 5′FITC-GTA AAA CGA CGG CCA GT 3′ were FITC is fluorocein isothiocyanate and Primer #BioM13R48 (SEQ ID NO: 16) has the sequence 5′ Bio-GGA TAA CAA TTT CAC ACA GGA 3′ where Bio is biotin. The primers were reconstituted in DI water to 0.1 mM (100 pm/pl) and stored in 2 ml screw top vial at −20° C. as a stock solution.

100 μl of the retentate was added to a 0.2 ml PCR tube. 2011 of Thermopol 10× buffer, 4 μl NTP stock solution, and 4 μl of 100 pmole/μl of each primer was added. The final volume was brought to 200 μl with DI H₂O. The samples were mixed and placed in PCR machine. When the temperature reaches 96° C. the program was pauses and 2 μl Deep Vent (exonuclease negative) DNA Polymerase stock solution (2 units/μl) (New England BioLabs cat #MO 259S) was added with 10× ThermoPol Reaction Buffer. 10× ThermoPol buffer comprises 10 mM KCL, 10 mM (NH4)₂SO₄, 20 mM Tris-HCL (pH 8.8, 2° C.), 2 mM MgSO4, and 0.1% Triton X-100. The reaction mixture was aliquoted into empty 50 μl PCR tubes preheated in the machine to 96° C. The total amplification time was about 2.5-3 hours.

The amplified DNA was purified by extraction with an equal volume of a phenol-chloroform-isoamyl Alcohol solution (25:24:1 v/v). 200 μl of the amplified DNA was transferred to a 1.5 ml Eppindorf tube. 200 μl of the extraction solution was added to the tube. The tube was swirled and then centrifuged for 5′/12,000 g. The supernatant (buffer layer) was aspirated and transferred to a new 1.5 ml Eppindorf tube.

The aspirated DNA solution undergoes salt precipitation using 100% ethanol. 100 μl of 8M Ammonium Acetate was added to ˜200 μl of the aspirated DNA. 2.6 times the combined (DNA+Ammonium Acetate) volume (˜780-800 μl) of cold absolute ethanol (−20° C.) was added to the tube. The tube was mixed and store in ice water for 30′. The sample was centrifuged for 15′/12,000 g. The supernatant was aspirated and discarded. 0.5 ml of 70% (V/V) ethanol was added and the sample was centrifuged for 5′/12,000 g. The supernatant was removed without disturbing the pellet and evaporate to dryness by exposing to air at RT. The pellet was reconstituted by adding 8 μL of a solution containing 4 μl of sterile DI H₂O+4 μl of 0.1 mM primer (F21-10-17). The sample is transferred to a 0.2 ml PCR tube and 2 μl of 5× TNKMg5 buffer is added. The surrogate antibody was reformed by the addition of excess F21-10-17 (−48 nt) primer favors the formation of the desired +78/−48 SAb molecules.

IV. Annealing the SAb

The dsSAb was annealed by heating the reconstituted material in a 0.2 ml PCR tube using the temperature program previously specified for annealing. 7 μl of 60% w/v sucrose with 10 μl of DNA and load sample onto a 16% acrylamide gel. Any DNA component with FITC at 5′ end (i.e. SAb 78/48, ds 48 and ss48) will appear on the gel as a green fluorescent band under long wavelength (UV-366 nm BLAK-RAY LAMP model UVL-56). The 5 pMol of F21-10-17 (−48 nt primer) was also run on the gel as a size marker. The SAb 78/48 will be observed to co-migrate with 500-600 nt dsDNA. The SAb-gel section was excised and mascerated and 250 μl of TNKMg5 Tw 0.05 buffer was added to the sample. The sample was then incubated for 2 hrs/RT while agitating on vortex at the lowest speed.

The gel suspension was transferred to an Amicon PCR Centrifugal Device and centrifuge at 1000 g/10′ to remove the polyacrylamide. The retentate was washed by adding a 50 μl aliquot of buffer, centrifuge at 1000 g/10′. The recovered SAb from the filtrate for use in subsequent selection cycle. The Sab was quantified by FU's using a 100 μl aliquot of the filtrate on the Wallac Victor2.

V. Selection Cycles 2-7

Negative selection using BSA was not performed in Cycle #1. The negative selection mixture comprises 2501 μl of SAb 78/48 filtrate (2-20 pMol by FITC) with 20 μl of a 1 μg/μl (20 μg) BSA solution. The sample was incubate for 30′/RT and the RFU's of 100 μl aliquot using Wallac VICTOR II was measured.

250 μl of the above reaction mix (20 μl is saved for 16% non-denaturing PAGE and 8% denaturing PAGE with 8M urea) is added to Nanosep 100K Centrifugal concentrator. The filter was centrifuged at 1000 g/15′/RT. The total volume in filtrate was ˜240 μl. The filtrate is aspriated and place in a new 1.5 ml Eppindorf tube. The RFU's of a 100 μl aliquot was determined.

The filter was washed by adding 200 μl TNKMg5 buffer, centrifuge (1000 g/10′/RT), and an additional 200 μl of same buffer was added after centrifugation. The sample was re-centifuged and 100 μl of same buffer was added. The sample was centrifuged again. 100 μl DI H₂O was added to filter and swirled and the retentate is aspirated. The RFU's was determined on Wallac VICTOR II of SAb bound to BSA by aspirating retentate and determining % recovery.

200 μl of negatively selected filtrate was mixed with 20 μl (1 μg/μl) of the BSA-aa-BZ10 conjugate suspended in TNKMg5 buffer. The sample was ncubated for 1 hour/RT. Total volume of the reaction is 220 μl.

The reaction solution was added to a new Nanosep 100K centrifugal device and centrifuged at 1000 g/15′. The filter was wash 3 time using TNKMg5 buffer. RFU's of a 100 μl aliquot of the filtrate was determined along with the % of unbound (free) SAb. 100 μl of DI H₂O was added to the filter, swirled, and the retentate aspirated. The entire sample was placed in a microtiter plate well and the RFU's and % recovery was measured.

From 1-20% of the bound SAb recovered in the 100 μl aliquot for PCR amplification was used with primer #BioM13R48 (100 pMol) and FM13-20 (100 pMol). This will again generate dsDNA in 4 tubes each containing 50 μl as described previously. Cycles of negative and positive selection are repeated until no further enrichment in % recovery is observed in the SAb population.

Additional cycles can be performed by preincubating the free hapten with the polyclonal SAb library prior to addition of the conjugate, and collecting the filtrate for subsequent amplification. A cycle(s) of affinity enhancement can be performed by incubating the SAb and conjugate in the presence of elevated MeOH, surfactant, decreased pH, and/or increased salt. High affinity SAb remaining bound to the conjugate was amplified. The process of Polyclonal SAb production proceeds through 1) binding, 2) specificity enhancement, and 3) affinity enhancement prior to production of monoclonal SAb clones.

VI. Calculations

The total amount of RFU's in the recovered conjugate-binding aliquot vs. the total amount of RFU's that were present when incubated with the conjugate represents the % of the surrogate antibody bound.

For negative selection, the amount of RFU's in the recovered BSA-binding aliquot vs. the total amount of RFUs present when incubated with BSA is determined.

Additional calculations include RFUs quantified from the filtrate that provides supportive data and information indicating unbound SAb and loss on filter device.

Further note that the DNA/conjugate and DNA/BSA ratios in cycles #2-5 was 10-100 nM DNA/2,000 nM protein, or 1 molecule of SAb 78/48 to 20-200 molecules of the conjugate or BSA. This calculation assumes that the conjugate has the reported 20 moles of BZ10 per mole of protein. In addition, the molecular weight of the (SAb 78/48-BSA-aa-BZ101) complex is about 113.4 Kd (A17-40-21=24 Kd)+(FM21-10-17=15.4 Kd)+(BSA=67 Kd)+(20 BZ101=7 Kd). The molecular weight of 2SAb:1 conjugate is ˜152.8 Kd and the molecular weight of 1SAb:2 conjugate ˜189.4 Kd.

C. Results

The production of surrogate antibody show in FIG. 1 was initiated to provide a more versatile core molecule than an aptamer having a stem-loop structure. The design incorporates constant region domains that bracket binding specificity domain. The multi-oligonucleotide structure allows for the simple attachment of multiple labels (e.g. FITC, biotin) that may, or may not be the same. Multiple, self-directed and self-forming, binding cavities can be readily incorporated. A stabilizing strand that is separate from the binding strand offers a convenient site for chemical modifications when required.

The surrogate antibodies are formed by annealing a “specificity-strand” to a “stabilizing-strand” prior to incubation with the target. Molecules that bind are amplified using asymmetric PCR that preferentially enriches the “specificity-strand”. The constant sequence “stabilizing-strand” is added, and surrogate molecules are annealed for another selection cycle.

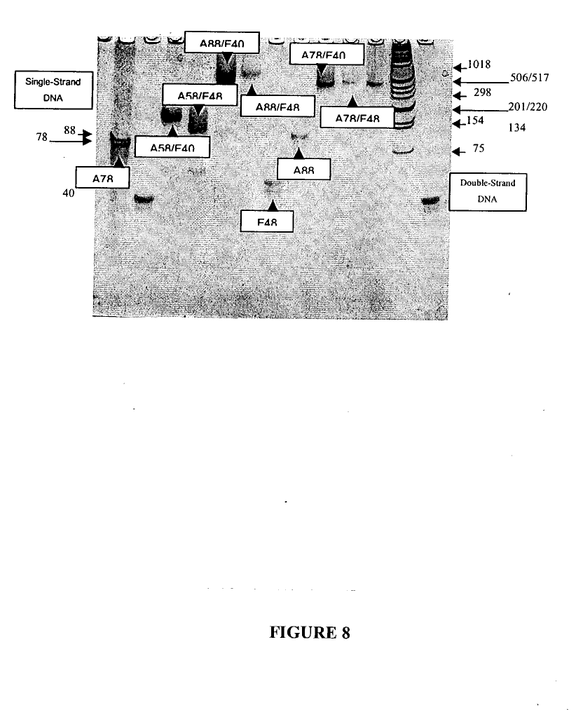

Surrogate antibodies can be assembled using “binding strands” that vary in the number of nucleotides in the binding loop. Each of these molecules will have a different binding cavity size and unique binding configurations. FIG. 8 illustrates the electrophoretic mobility of the surrogate antibodies that were assembled using different combinations of “specificity” and “stabilizing” primers. Fluorocein-labeled “stabilizing strands” (prefix “F”) and un-labeled “specificity strands” (prefix “A”) were used in the production of these molecules. This combination illustrates a significant shift in the electrophoretic mobility of the fluorocein-labeled “Stabilization” strand and the annealed molecule.

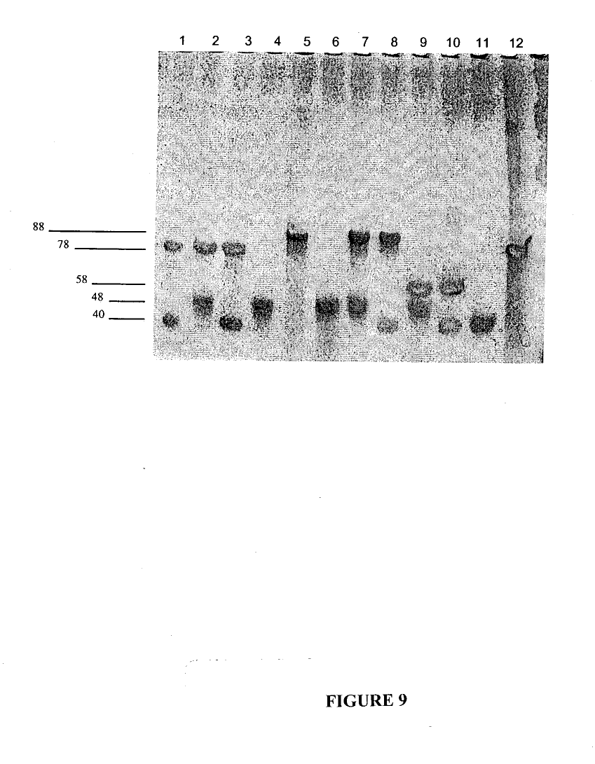

The surrogate antibodies were characterized using non-denaturing acrylamide gel electrophoresis were re-characterized using a denaturing gel (8% acrylamide, 8M urea) to verify the duplex nature of the molecule and approximate 1:1 stoichiometry of the “specificity” and “stabilization” strands (FIG. 9).

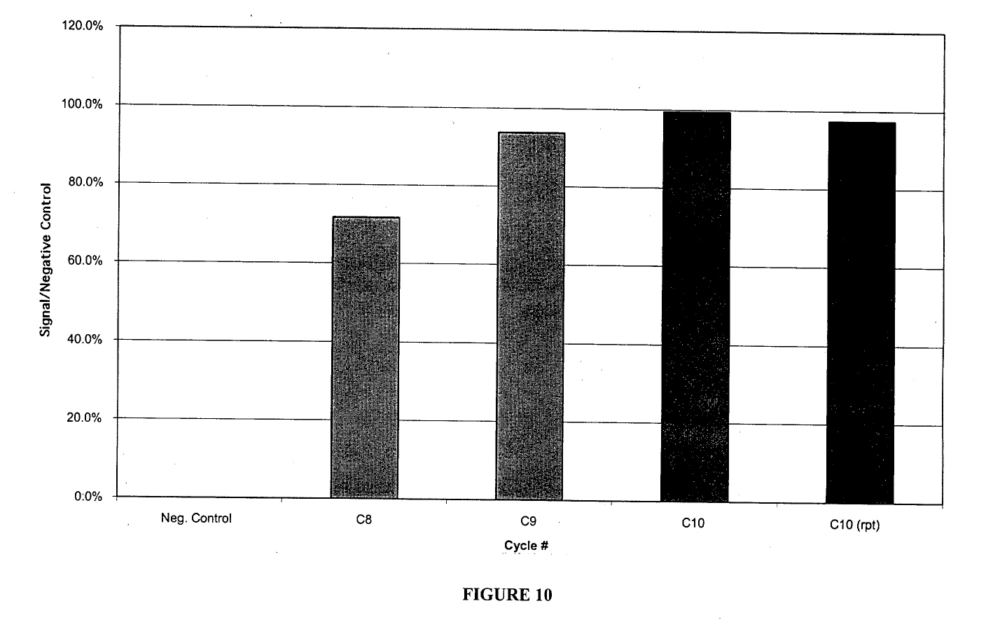

FIG. 10 illustrates the selection and enrichment of the surrogate antibodies to the BSA-PCT (BZ11 congener) conjugate through 8, 9 and 10 cycles. Signal/Negative control represents as a percent the amount of surrogate antibody bound to the target verses the amount of surrogate antibody recovered when the target is absent (negative control).

D. Observations and Conclusions

The surrogate antibody binding affinity for the non-polar BZ101 congener is believed to be the result of the binding loop/cavity designed into the molecules and hydrophobic interactions. The observation is similar to other experiments that illustrated the high affinity binding of PCB congeners by β cyclodextrins. The better than expected sensitivity obtained may also suggest the cooperative effect of hydrophobic, hydrogen, electrostatic and Van der Waals bonds. The binding of the BZ11-BSA conjugate, and the effective inhibition of binding induced by relatively low concentrations of free BZ101, was of special interest. The data suggests limited preferential binding of the conjugated ligand that was used during selection, and that the same bridge chemistry could be used in a reporter molecule for final immunoassay. This is typically not an available option when developing a hapten-specific immunoassay, where preferential antibody binding, and decreased assay sensitivity, would occur if the reporter molecule and immunogen shared the same bridge chemistry. The observation illustrates the versatility of the selection method and ability to eliminate bridge and carrier binding molecules from the SAb library. The data demonstrates the rapid production of a new binding reagent that could preferentially bind an EPA-specified PCB congener at a concentration below the regulatory action limit.

### EXAMPLE 7

### Use of Surrogate Antibodies in Arrays

Five monoclonal surrogate antibody reagents to the congeners designated in Table 1 will be prepared for the Aroclor® immunoassay array. A ample will be produced that will allow the testing of complex PCB samples that contain oils or solvents.

TABLE 1
5 Congeners of Interest

| | | | M.W. |
|---|---|---|---|
| 2,2′,3,4,4′5,5′ Heptachlorobiphenyl | BZ180 | C₁₂H₃C₁₇ | 395.35482 |
| 2,3,3′,4′,6 Pentachlorobiphenyl | BZ110 | C₁₂H₅C₁₅ | 326.4567 |
| 2,2′4,5,5′ Pentachlorobiphenyl | BZ101 | C₁₂H₅C₁₅ | 326.4567 |
| 2,3′4,4′ Tetrachlorobiphenyl | BZ66 | C₁₂H₆C₁₄ | 292.00764 |
| 2,2′5 Trichlorobiphenyl | BZ18 | C₁₂H₇C₁₃ | 257.55858 |

Five immunoassays, each targeting one of the Method 8082-specified congeners, will be developed. The unique response profile produced by the five tests will be used to identify the Aroclor present. The composite signal generated will be used to quantify Aroclor concentration. A single well “total PCB” assay will be formulated using a polyclonal reagent from the five monoclonal surrogate antibodies produced.

Proposed Test Characteristics:

Aroclor® composition data published by Frame (Frame et al (1997) Anal. Chem 468A-475A) and EPA Region V (Frame et al. (1996) J. High Resol. Chromatogr 19:657-688) were used to select target congeners that would collectively provide a unique, predictable, and detectable response profile. Table 2 illustrates the weight % composition of the congeners in each of five EPA-specified Aroclors.

TABLE 2
Weight % Composition of Selected Congener in Five Aroclors ®

Congener Wt. % in Designated Aroclor

| | 180 | 110 | 101 | 66 | 18 |
|---|---|---|---|---|---|
| molecular weight | 395.35 | 326.46 | 326.46 | 292.01 | 257.56 |
| 1260 | 11.38 | 1.33 | 3.13 | 0.02 | 0.05 |
| 1254 (composite) | 0.55 | 8.86 | 6.76 | 2.29 | 0.17 |
| 1248 (composite) | 0.12 | 2.76 | 2.06 | 6.53 | 3.79 |
| 1242 | 0.00 | 0.83 | 0.69 | 3.39 | 8.53 |
| 1016 | 0.00 | 0.00 | 0.04 | 0.39 | 10.86 |

Table 3 illustrates the molar concentration of each congener when the total Aroclor® concentration in a sample is 10 ppm, the EPA-OSWER regulatory action level for solid-waste.

TABLE 3
Molar concentration of congeners in a sample when total Aroclor ®
concentration of the sample is 10 ppm.

Molar Concentration of Congener in Sample when
Total Aroclor Concentration In Sample = 10 ppm

| | 180 | 110 | 101 | 66 | 18 |
|---|---|---|---|---|---|
| 1260 | 2.88E−06 | 4.07E−07 | 9.59E−07 | 6.85E−09 | 1.94E−08 |
| 1254* | 1.38E−07 | 2.71E−06 | 2.07E−06 | 7.83E−07 | 6.41E−08 |
| 1248* | 2.91E−08 | 8.45E−07 | 6.29E−07 | 2.24E−06 | 1.47E−06 |
| 1242 | 0.00E+00 | 2.54E−07 | 2.11E−07 | 1.16E−06 | 3.31E−06 |
| 1016 | 0.00E+00 | 0.00E+00 | 1.23E−08 | 1.34E−07 | 4.22E−06 |

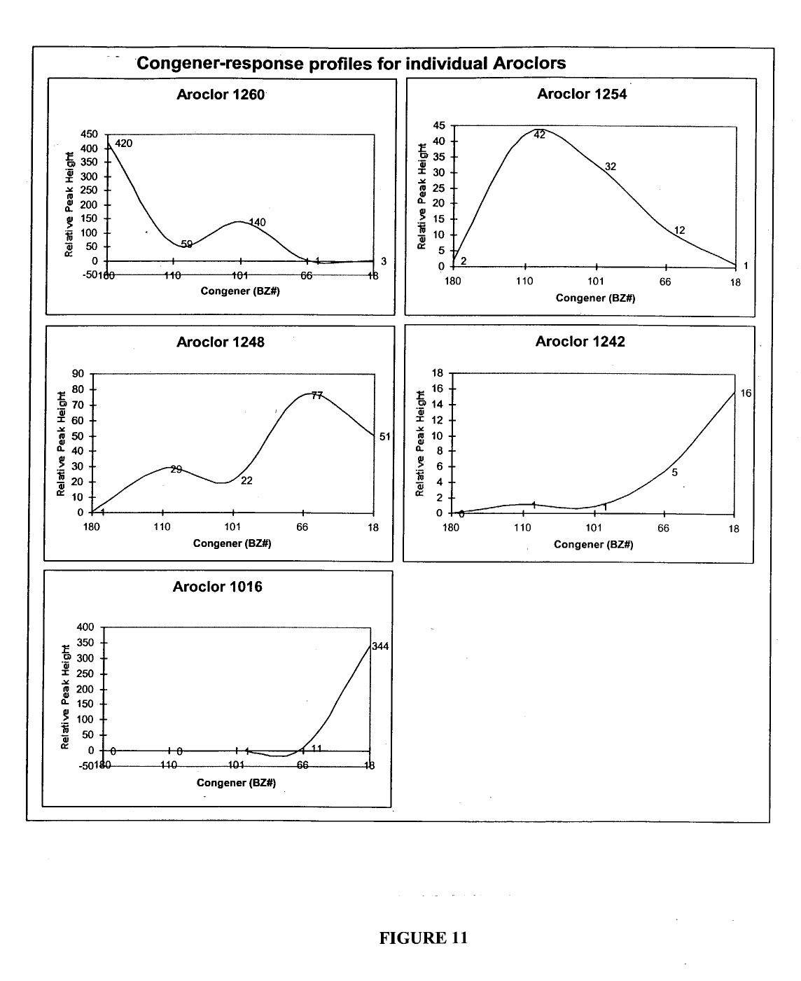

This concentration approximates the Ka each of the immunoassays and surrogate antibody would need to achieve to detect the congener in the middle (B₅₀) of their respective dose-response curves. Some of the cited applications for the test will require a practical quantitation limit of 2 ppm, a concentration that would require 2-4 times greater affinity. Based upon the BZ101 immunoassay data generated and the literature cited for the affinity of aptamers, immunoassays developed using surrogate antibodies should achieve the required practical detection limits without additional pre-analysis concentration steps. Table 4 indicates the relative distribution of the selected congeners in each of the Aroclors, and FIG. 11 illustrates the unique congener response profiles the array would produce for selected Aroclors®.

TABLE 4
Relative Peak Heights of Congeners in Specified Aroclors ®

| | Ratio of Peak Heights at 10 ppm Aroclor Concentration | | | | |
|---|---|---|---|---|---|
| | 180 | 110 | 101 | 66 | 18 |
| 1260 | 420 | 59 | 140 | 1 | 3 |
| 1254* | 2 | 42 | 32 | 12 | 1 |
| 1248* | 1 | 29 | 22 | 77 | 51 |
| 1242 | 0 | 1 | 1 | 5 | 16 |
| 1016 | 0 | 0 | 1 | 11 | 344 |

*average of “a” and “g”

Surrogate Antibody Development:

The five congeners identified in Table 1 for surrogate antibody development were selected on the basis of;

1. concentration compatible with the anticipated surrogate antibody binding constant (note; the sample processing chemistry developed would allow the PCBs to be concentrated and thereby overcome a disparity between binding Ka and required assay detection range.)

2. unique Aroclor® distribution profile (note; the unique response profile of the immunoassays will be used to Aroclors® in the way the gas chromatography reference method is used)

3. their citation in EPA reference Method 8082

4. congeners having an approximately equal concentration in Aroclor 1248a and 1248g, and 1254a and 1254g (note; the first generation product will not differentiate these sub-populations)

Surrogate antibody molecules will be assembled before each selection cycle into duplex oligonucleotides having one strand that may be unlabeled or labeled using a biotin-primer, and the other strand labeled with fluorocein isothiocyanate (FITC) at the 5′ end (Kato et al. (2000) NAR 28:1963-1968). A Wallac Victor 2 multi-label reader will be used to quantify the concentration of the FITC-labeled strand and assembled SAb. Non-denaturing acrylamide gel (16%) will be used to confirm the assembly of SAb's by noting the change in mobility of the unannealed vs. annealed FITC-labeled strand. Electrophoresis using 8% acrylamide gel and 8M urea will be used to confirm that the identity of the annealed duplex molecule. Yield and % recovery of the assembled SAb will be quantified by determining the amount of SAb related fluorescence in an excised SAb gel fraction to the total fluorescence of the components.

The initial unselected population will be incubated with a congener-BSA conjugate to produce an amplified binding population. The “size-exclusion” filtration method, using the Microcon® device will be used to separate SAb molecules bound to the conjugate from those not bound. Unbound molecules will pass into the filtrate. Volume and fluorescence will be quantified and the fraction discarded. Molecules in the retentate will similarly be quantified for volume and fluorescence and then used for PCR amplification. The relative amount of fluorescence in the retentate vs. total starting fluorescence will be calculated as % recovery (% bound/total).

PCR will be performed using two primers, one labeled with FITC. The FITC primer will be used to produce the positive congener-binding strand. Standard PCR will be performed using 40 cycles of amplification, Deep-Vent® polymerase (exonuclease free), and NTPs. PCR products will be purified with phenol/chloroform extraction and NaAc:EtOH precipitation to remove proteins (e.g. polymerase) and to concentrate the product. The “Stabilizing” primer (with/without biotin) will be added to the “binding” strand of the purified PCR pellet at a 4-10 molar excess concentration. The mixture will be annealed using a thermal cycler at 95° C./5′, 65°/20′, 60°/5′, 55°/5′, and then cooled to RT at the rate of 1°/1′. The 65° C. annealing temperature is used to favor the formation of duplex SAb's that have Tm's in the 80° C. range. Sucrose buffer (7 μl, 60%) will be added to the SAb's to increase density prior to electrophoresis. Non-denaturing electrophoresis (16% acrylamide, 100V, RT) will be used to fractionate the SAb from other components. The FITC-labeled SAb will be located on the gel by fluorescent scanning and mobility (Rf) and excised for use in selection. SAb will be extracted from the macerating gel after the addition of a buffer, incubation for 2 hours, and Microcon® filtration.

The congener-BSA conjugate will first be filtered through a Microcon® column. Conjugate appearing in the filtrate will be discarded and conjugate in the retentate recovered for use in the selection. The processed conjugate (10-20 μl) will be incubated with the purified SAb and incubated at RT/60′. The incubated solution will be filtered and SAb in the retentate recovered, quantified for FITC, and amplified. The % bound/total SAb will again be calculated. Incubation with exonuclease I will be used to demonstrate the formation and use of the duplex structure (note; SAb molecule should be resistant to degradation by this enzyme). Selection cycles will continue until further enrichment in % B/T is not produced.

Specificity enrichment will remove surrogate antibodies that recognize the derivatized BSA carrier. The enriched binding population will undergo cycles of incubation with unconjugated BSA followed by Microcon® filtration. The non-specific oligonucleotides in the retentate will be discarded and those in the filtrate will be re-processed until base-line protein binding is obtained. Similar cycling will be performed by adding methanol extracts of negative soil samples prior to the addition of the target conjugate. Surrogate antibodies bound to the conjugate will be recovered for amplification. A final cycle of incubation using the unconjugated target congener, filtration, and amplification of SAb in the filtrate, will provide a polyclonal reagent free of derivative recognition. The consistent use of 10% MeOH in the selection buffers will enhance affinity and allow for higher PCB concentrations to be achieved in the final immunoassay. Published data on the use of MeOH indicates limited destabilization of a double helix relative to water (Albergo et al. (1981) Biochem 20:1413-8) suggesting that hydrophobic bonds are not a major component of duplex stability (Hickey et al. (1985) Biochem 9:2086-94)

Monoclonal surrogate antibodies will be produced from the enriched polyclonal reagent. Molecules having a single deoxyadenosine (A) at the 3′ end will be ligated using a pGEM-T EASY Vector® System (Promega). One sequence insert will ligate into each vector and produce individual bacterial colonies that have a single sequence. The presence of α-peptide in the vector sequence allows direct color screening of the recombinant clones on indicator plates. Clones containing the PCR fragments will produce white or light blue colonies. The PCR amplification and annealing protocols previously used will again be used to produce individual wells that contain monoclonal surrogate antibody. Each well will next be characterized.

Characterization and Method Development:

Black microplates, suitable for fluorescence detection, will be passively coated with the congener-BSA conjugate used for selection. Conjugates will be modified to alter the location or number of chlorine atoms if preferential conjugate binding of the SAb is observed. Standard validation protocols will be used to select molecules on the basis of affinity, congener cross-reactivity, cross-reactivity to related compounds or others that may be present, and matrix interferences. A database will be prepared to compare the performance of the SAbs and select one or more for use in the array. The performance advantage, if any, obtained by combining multiple monoclonal reagents into a polyclonal reagent for the test will be reviewed and considered. Selected surrogate antibody molecules will be sequenced and then synthesized to provide needed array-development material.

The characterization method will rely on detecting single, or double, FITC-labeled surrogate antibody molecules. The immunoassay protocol will incubate, in solution, surrogate antibody molecules with standards, samples, or controls. The reaction mixture will be added to microtiter plate wells coated with the target conjugate and blocked with 2% BSA. After 15-30 minutes the contents will be removed and the wells washed with a buffer containing Tween® 20. The signal will be quantified using a Wallac Victor II multi-label reader. Surrogate antibody titers will be quantified by testing doubling dilutions in 10% MeOH-Tris HCl buffer Dose-response characteristics will be calculated using an assay composed of a surrogate antibody dilution and 10 ppm congener illustrating 50% binding inhibition (B₅₀/ED₅₀). Dose-response curves will be produced using 5 congener standards. The curve will be linearized using a logit-log transform of the data to allow y=m×+b extrapolation of the data. The quantitation range of the competitive binding assay will typically extends from B₈₀ (i.e. 80% conjugate binding) to B₂₀ (20% Binding). The concentration range will span one to two logs depending upon the Ka of the surrogate antibody. The linearity of standard curves will be assessed from the correlation coefficient of the logit-log line (r²). Standard curves with a correlation coefficient ≧0.95, and % error of the duplicate standards ≦15%, will be used for calculating validation parameters (e.g. sensitivity, % cross-reactivity).

Preliminary % cross-reactivity will define the concentration of the non-target congeners needed to inhibit 50% of the surrogate antibody binding to the target congener. This ratio will be expressed as the % cross-reactivity. To develop an array, antibody with <10% cross-reactivity will be selected. Similar studies will be performed using the compounds listed on the “specifications sheet” as possible cross-reactants. Spike-recovery studies using various sample matrices will evaluate relative matrix effects. Sensitivity, expressed as least detectable dose (LDD), minimum detection limit (MDL), practical quantitation limit (PQL) will be calculated as the extrapolated congener concentration equal to a multiple (e.g. LDD=2σ) of the signal standard deviation obtained from the simultaneous testing of multiple negative samples. Aroclors® will be tested at concentrations ≦10 ppm to verify detection capability and consistency with the anticipated response profiles (FIG. 11).

Surrogate antibody reagents for detecting each of the congeners will be combined and used with a microtiter plate having the five conjugates immobilized in adjacent wells. Unconjugated BSA will be immobilized to separate wells and used as a control. The assay will be used to test Aroclor® standards and spiked matrices. Profile array data will be collected and peak height vs. Aroclor correlation studies performed and collected. A total PCB, as opposed to an Aroclor identification assay format, will be evaluated by immobilizing a mixture of the 5 congener conjugates to individual microtiter wells. Samples will be incubated with the mixture surrogate antibody reagents and added to the mixed conjugate wells and BSA control wells. Standard FDA and EPA validation protocols will be performed to assess preliminary sensitivity, cross-reactivity, matrix interferences, and % recovery characteristics.

### EXAMPLE 8

### Methods for Making a Ligand-Binding Surrogate Antibody Reagent that Recognizes IgG

As outlined in Example 5, surrogate antibody (SAb) molecules were produced using self-assembling oligonucleotide strands (87 nt+48 nt) to form a dimeric molecule having a 40 nt random specificity domain sequence with adjacent constant nucleotide sequences. Cycles of ligand binding, PCR amplification, bound/free separation, and reassembly/reannealing were used to enrich the SAb population with molecules that would bind an IgG polypeptide. Methods for the selection are discussed in detail in Example 1.

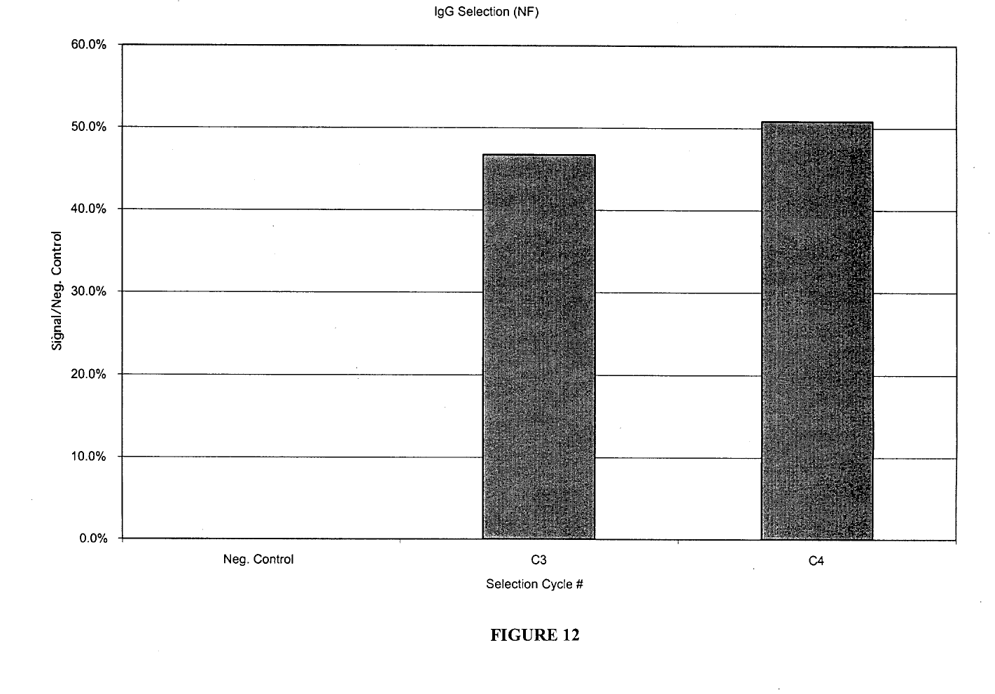

FIG. 12 illustrates the selection and enrichment of the surrogate antibodies to IgG. Signal/Negative control represents as a percent the amount of surrogate antibody bound to the target verses the amount of surrogate antibody recovered when the target is absent (negative control).

The following references are incorporated herein in their entirety for all purposes.

Ono et al. (1997) Nucleic Acids Research 25(22): 4581-4588

Peyman et al. (1996) Biol Chem Hoppe Seyler, 377(1): 67-70

Khan et al. (1997) J. Chrom. Biomed. Sci. Appl. 702(1-2):69-76

Maier et al. (1995) Biomed Pept Proteins Nucleic Acids 1(4):235-42

Boadoetal. (1992) Bioconjug Chem 6:519-23

Jayasena et al. (1999) Clin Chem 45;9:1628-1650

Dougan et al. (2000) Nucl Med Biol 27(3):289-97

Brody et al. (2000) J. Biotech.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

That which is claimed:

1. An isolated molecule comprising a specificity strand and a stabilization strand,
said specificity strand comprising a nucleic acid sequence having a specificity region flanked by a first constant region and a second constant region;
said stabilization strand comprises a first stabilization domain that interacts with said first constant region and a second stabilization domain that interacts with said second constant region; and,
said stabilization strand and said specificity strand comprise distinct molecules.

2. The isolated molecule of claim 1, wherein said stabilization strand further comprises a first spacer domain between said first stabilization domain and said second stabilization domain.

3. The isolated molecule of claim 1, wherein said stabilization strand comprises an amino acid sequence.

4. The isolated molecule of claim 1, wherein said nucleic acid sequence comprises a deoxribonucleic acid sequence or a ribonucleic acid sequence.

5. The isolated molecule of claim 1, wherein said molecule further comprises at least one functional moiety.

6. The isolated molecule of claim 1, wherein said specificity region binds a ligand.

7. The isolated molecule of claim 1, wherein said stabilization strand comprises a second nucleic acid sequence.

8. The isolated molecule of claim 7, wherein at least one of said nucleic acid sequence or said second nucleic acid sequence comprises a deoxyribonucleic acid sequence or a ribonucleic acid sequence.

9. The isolated molecule of claim 7, wherein the second nucleic acid sequence comprising said stabilization strand is at least 8 nucleotides.

10. The isolated molecule of claim 7, wherein said specificity strand comprises at least 10 nucleotides.

11. The isolated molecule of claim 7, wherein said molecule binds a ligand.

12. The isolated molecule of claim 11, wherein said ligand comprises a polypeptide, a nucleotide, a chemical compound, a mucopolysacharide, a cell, an organism, a bacteria, a virus, a lipid, an inorganic molecule, an organic molecule or a PCB.

13. The isolated molecule of claim 12, wherein said polypeptide is a receptor.

14. The isolated molecule of claim 7, wherein said molecule acts as a ligand.

15. The isolated molecule of claim 7, wherein said molecule further comprises at least one functional moiety.

16. The isolated molecule of claim 15, wherein said functional moiety comprises a reporter molecule, an affinity type molecule, a linking molecule, or an enzyme.

17. The isolated molecule of claim 15, wherein said functional moiety is an organic molecule or an inorganic molecule.

18. The isolated molecule of claim 15, wherein said functional moiety is a therapeutic agent.

19. The isolated molecule of claim 18, wherein said therapeutic agent is an anti-microbial agent having anti-microbial activity.

20. The isolated molecule of claim 19, wherein said anti-microbial activity comprises anti-bacterial activity, anti-viral activity, or anti-fungal activity.

21. The isolated molecule of claim 15, wherein said functional moiety comprises at least one modified nucleotide.

22. The isolated molecule of claim 15, wherein said functional moiety is located in said specificity region.

23. The isolated molecule of claim 22, wherein said functional moiety introduces hydrophobic binding capabilities into said specificity region.

24. The isolated molecule of claim 15, wherein said functional moiety comprise a modified nucleotide having a modification at the 2′ position of the nucleotide sugar or phosphate molecule.

25. The isolated molecule of claim 15, wherein said functional moiety increases resistance to nuclease degradation.

26. The isolated molecule of claim 15, wherein said functional moiety is located in said stabilization strand.

27. The isolated molecule of claim 26, wherein said functional moiety comprises a non-amplifiable moiety that increases resistance to polymerase activity in a PCR reaction.

28. The isolated molecule of claim 1, wherein
said specificity strand further comprises a second specificity region flanked by said second constant region and a third constant region; and,
said stabilization strand further comprises a third stabilization domain that interacts with said third constant region.

29. The isolated molecule of claim 28, wherein said stabilization strand further comprises a first spacer region between said first stabilization and said second stabilization domain and a second spacer region between said second stabilization domain and said third stabilization domain.

30. The isolated molecule of claim 28, wherein said stabilization strand comprises a nucleic acid sequence.

31. The isolated molecule of claim 28, wherein said stabilization strand comprises an amino acid sequence.

32. A composition comprising a population of molecules of claim 1.

33. A library of isolated molecules comprising:
a population of molecules comprising a specificity strand and a stabilization strand, said specificity strand comprising a nucleic acid sequence having a specificity region flanked by a first constant region and a second constant region; and, said stabilization strand comprises a first stabilization domain that interacts with said first constant region and a second stabilization domain that interacts with said second constant region; and,
wherein each of the first constant region of said specificity strands in said population are identical; each of the second constant region of said specificity strands in said population are identical; and, each of the specificity region of said specificity strands in said population are randomized; and,
wherein each of the stabilization strands in said population are identical.

34. The library of claim 33, wherein said stabilization strand and said specificity strand comprise distinct molecules.

35. A library of isolated molecules produced by
a) providing a population of specificity strands wherein
i) each of said specificity strand in said population comprises a nucleic acid sequence having a specificity region flanked by a first constant region and a second constant region;
ii) each of the first constant region of said specificity strands in said population are identical;
iii) each of the second constant region of said specificity strands in said population are identical; and,
iv) each of the specificity region of said specificity strands in said population are randomized; and,
b) contacting said population of specificity strands with a stabilization strand;
wherein said stabilization strand comprises a first stabilization domain that interacts with said first constant region and a second stabilization domain that interacts with said second constant region; and,
said contacting occurs under conditions that allow for said first stabilization domain to interact with said first constant region and said second stabilization domain to interacts with said second constant region.

36. The library of claim 35, wherein said stabilization strand and said specificity strand comprise distinct molecules.

37. A method for generating a surrogate antibody library comprising:
a) providing a population of specificity strands wherein
i) each of said specificity strand in said population comprises a nucleic acid sequence having a specificity region flanked by a first constant region and a second constant region;
ii) each of the first constant region of said specificity strands in said population are identical;
iii) each of the second constant region of said specificity strands in said population are identical; and,
iv) each of the specificity regions of said specificity strands in said population are randomized; and,
b) contacting said population of specificity strands with a stabilization strand;
wherein said stabilization strand comprises a first stabilization domain that interacts with said first constant region and a second stabilization domain that interacts with said second constant region; and,
said contacting occurs under conditions that allow for said first stabilization domain to interact with said first constant region and said second stabilization domain to interacts with said second constant region.

38. The method of claim 37, wherein said stabilization strand and said specificity strand comprise distinct molecules.

39. A method for capturing a surrogate antibody comprising:
a) contacting a ligand with a population of surrogate antibody molecules under conditions that permit formation of a population of ligand-bound surrogate antibody complexes, wherein each of the surrogate antibody molecules of the surrogate antibody population comprises a specificity strand and a stabilization strand,
said specificity strand comprising a nucleic acid sequence having a specificity region flanked by a first constant region and a second constant region;
said stabilization strand comprises a first stabilization domain that interacts with said first constant region and a second stabilization domain that interacts with said second constant region;
b) partitioning said ligand and said population of surrogate antibody molecules from said population of ligand-bound surrogate antibody complexes; and,
c) amplifying the specificity strand of said population of ligand-bound surrogate antibody complexes.

40. The method of claim 39, wherein said population comprises a library of surrogate antibody molecules.

41. The method of claim 39, wherein said stabilization strand and said specificity strand comprise distinct molecules.

42. The method of claim 41, wherein said population comprises a selected population of surrogate antibodies.

43. The method of claim 39, wherein said method further comprises contacting said population of specificity strands of step (c) with a stabilization strand under conditions that allow for said first stabilization domain to interact with said first constant region and said second stabilization domain to interact with said second constant region.

44. The method of claim 39, wherein said method further comprises isolating a cloned specificity strand said isolating comprising cloning at least one specificity strand of the amplified population of specificity strands of step (c).

45. The method of claim 44, wherein said method further comprises contacting said cloned specificity strand with the stabilization strand under conditions that allow for said first stabilization domain to interact with said first constant region and said second stabilization domain to interact with said second constant region.

46. The method of claim 39, wherein partitioning comprises filtering said ligand, said population of surrogate antibody molecules, and said population of ligand-bound surrogate antibody complexes through a membrane having a porosity that retains the ligand-bound surrogate antibody complex in the retentate and allows unbound surrogate antibodies to pass into the filtrate.

47. A method of detecting a ligand comprising
a) contacting said ligand with a surrogate antibody molecule under conditions that permit formation of a population of ligand-bound surrogate antibody complexes, wherein said surrogate antibody molecule comprises a specificity strand and a stabilization strand,
said specificity strand comprising a nucleic acid sequence having a variable region flanked by a first constant region and a second constant region;
said stabilization strand comprises a first stabilization domain that interacts with said first constant region and a second stabilization domain that interacts with said second constant region; and,
b) detecting said ligand.
